(19) United States
(12) Patent Application Publication
(10) Pub. No.: US 2018/0282690 A1
(43) Pub. Date: Oct. 4, 2018

(54) METHOD AND CULTURE MEDIUM FOR EX VIVO CULTURING OF EPIDERMIS-DERIVED STEM CELLS

(71) Applicant: Max-Planck-Gesellschaft zur Förderung der Wissenchaften e.V., München (DE)

(72) Inventors: Sara Annika WICKSTRÖM, Köln (DE); Carlos Andrés CHACÓN MARTINEZ, Köln (DE)

(21) Appl. No.: 15/765,779

(22) PCT Filed: Oct. 4, 2016
(86) PCT No.: PCT/EP2016/073675

§ 371 Date: Apr. 4, 2018

(30) Foreign Application Priority Data
Oct. 5, 2015 (EP) 15188393.1

(51) Int. Cl. C12N 5/071; C12N 5/095

(52) U.S. Cl. CPC C12N 5/0628; C12N 2533/90; C12N 2501/115; C12N 2501/117; C12N 2501/11; C12N 2500/46; C12N 2500/24; C12N 2502/1323; C12N 2506/094; C12N 2513/00; C12N 2501/727; C12N 2501/41; C12N 2501/165; C12N 2501/119; C12N 2533/52; C12N 2533/54; C12N 5/0695

(57) ABSTRACT

The present invention relates to a method for culturing epidermis-derived stem cells comprising the step of culturing epidermis-derived stem cells in the presence of a three-dimensional extracellular matrix (3D-ECM) and a basal cell culture medium comprising: Epidermal Growth Factor (EGF); and/or a Vascular Endothelial Growth Factor (VEGF); and/or a Fibroblast Growth Factor (FGF); and further a ROCK (Rho-kinase) inhibitor. The present invention further relates to a method for ex vivo de novo generation of epidermis-derived stem cells. Furthermore, the present invention relates to an epidermis-derived stem cell that is obtainable by a method according to the present invention. Uses of said epidermis-derived stem cell, e.g. uses of said epidermis-derived stem cell for in vitro tissue production, in vitro drug discovery screening and medical applications, are also provided herein. The present invention further relates to a cell culture medium that is employed in the context of a method of the present invention.

The present invention relates to a method for culturing epidermis-derived stem cells comprising the step of culturing epidermis-derived stem cells in the presence of a three-dimensional extracellular matrix (3D-ECM) and a basal cell culture medium comprising: Epidermal Growth Factor (EGF); and/or a Vascular Endothelial Growth Factor (VEGF); and/or a Fibroblast Growth Factor (FGF); and further a Rho-kinase (ROCK) inhibitor. The present invention further relates to a method for ex vivo de novo generation of epidermis-derived stem cells. Furthermore, the present invention relates to an epidermis-derived stem cell that is obtainable by a method according to the present invention. Uses of said epidermis-derived stem cell, e.g. uses of said epidermis-derived stem cell for in vitro tissue production, in vitro drug discovery screening and medical applications, are also provided herein. The present invention further relates to a cell culture medium that is employed in the context of a method of the present invention.

Adult somatic stem cells (SCs) fuel tissue renewal, repair, and remodeling in mature organs. By tuning their proliferation rate to match the changing needs of their resident tissues, SCs maintain organ form and function.

Mammalian skin is characterized by a hair coat that maintains body temperature, homeostasis and serves a protective function. During postnatal life, mammalian skin and its hair coat are constantly renewed. SCs residing in the epidermis (referred to as epidermis-derived stem cells) such as interfollicular epidermal SCs or hair follicle stem cells (HFSCs) ensure the maintenance of adult skin homeostasis and/or trigger hair regeneration. Moreover, both SCs residing in the epidermis and within the hair follicles (HFs) participate in the repair of the epidermis after injuries (Bianpain & Fuchs, 2014, Science 344, 1242281).

In particular, HFSCs, one important class of epidermis-derived stem cells, have the function of triggering the renewal of the hair coat. The constant renewal of the hair coat occurs by renewing the lower portion of the existing HFs in cycling phases of growth (anagen), regression (catagen), and rest (telogen) (Paus & Cotsarelis, 1999, N Engl J Med 341: 491-497). The continuous self-renewal of the HF is fueled by HFSCs residing in the specific niche referred to as bulge region that locates to the base of the non-cycling part of the HF. During telogen these HFSCs exist in a quiescent, slow-cycling state. At the onset of anagen they are activated to proliferate, expand, leave the niche and finally to differentiate in order to generate a new HF. Tight regulation of HFSC quiescence and activation is required to maintain a stable pool of stem cells enabling HF cycling throughout the lifetime of an individual (Blanpain & Fuchs, 2009, Nat Rev Mol Cell Biol 10: 207-217).

SCs are also found in epidermal tumor tissue and contribute to tumor development and growth. These epidermis-derived SCs, which are typically referred to as epidermis-derived cancer stem cells (CSCs) possess characteristics associated with normal epidermal SCs and are specifically characterized by the ability to self-renew i.e. to generate new CSCs as well as to give rise to all cell types found in a particular epidermis-derived tumor. To date, CSCs remain poorly characterized and no methods to specifically culture, maintain and/or expand these cells in vitro have been reported (Thieu et al., 2013, Cancer Lett 10, 82-88).

Studying the precise molecular mechanisms that regulate SCs and in particular epidermis-derived SC quiescence and activation requires inter alia good in vitro culturing methods, allowing for maintaining, expanding and/or manipulation of these cells.

Recently, cell culture systems that support survival and expansion of SCs from mouse and human epithelial tissues other than the epidermis have been reported. These tissues include: intestine (Sato et al., 2009, Nature 14: 262-265), colon (Sato et al., 2011, Gastroenterology 141: 1762-1772), stomach (Barker et al., 2010, Cell Stem Cell 5: 175-181), lung (Lee et al., 2014, Cell 156: 440-455), pancreas (Huch et al., 2013, EMBOJ 16: 2708-2721), and liver (Huch et al., 2013, Nature 14: 247-250) among others. For example, intestinal SC cultures are known as mini-gut or organoid cultures and can be established from a single Lgr5-expressing SC. In these organoid cultures SCs generate intestinal crypt-like structures that resemble the functionality and cellular heterogeneity of a normal gut. Similar to tissues/organs, these organoids contain a large proportion of differentiated cells in addition to SCs and they form anatomically distinct structures such as crypt-like structures.

WO 2010/090513 A2 describes a method for culturing of epithelial stem cells and isolated tissue fragments comprising epithelial SCs. In particular, the method described in WO 2010/090513 A2 is a method for culturing epithelial stem cells or tissue fragments comprising epithelial stem cells, wherein said stem cells or tissue fragments originate from the intestine, colon, stomach or pancreas but not from the epidermis. The disclosed culturing method involves the use of an extracellular matrix and a special cell culture medium, which comprises inter alia a Bone Morphogenic Protein (BMP) inhibitor and a mitogenic growth factor. The culturing method results in organoid structures. Accordingly, this method is a method for in vitro generation and/or culturing of organoids that comprise stem cells and a number of differentiated cells that form distinct anatomical structures. In particular, the culturing conditions are adapted to epithelial stem cells from the intestine, colon, stomach or pancreas but have not been used for culturing epidermis-derived stem cells. Besides these methods for culturing epithelial stem cells other than epidermis-derived stem cells, also methods for culturing epidermis-derived SCs have been described in the art. However, known culturing methods have certain disadvantages and/or limitations as further discussed below.

Most commonly, epidermis-derived SCs are cultured with in vitro culturing methods that have been described for culturing primary epidermal keratinocytes and also HF-derived keratinocytes (Barrandon & Green, 1987, Cell 50: 1131-1137, Blanpain et al., 2004, Cell 118: 635-648, Lien et al., 2014, Nat Cell Biol 16: 179-190, Ouji et al, 2015, J Invest Dermatol 135: 1598-1608, Trempus et al., 2003, J Invest Dermatol 120: 501-511, Bilousova & Roop, 2013, Methods Mol Biol, 961: 337-350, Jensen et al., 2010, Nat Protoc 5: 898-911, Lichti et al., 2008, Nat Protoc 3: 799-810, Oh et al., 2013, J Invest Dermatol 133: e14 doi:10.1038/jid.2013.387). Such methods typically involve culturing primary epidermal keratinocytes and also HF-derived keratinocytes comprising epidermis-derived stem cells on a fibroblast feeder-layer in a growth medium with low serum content, in low calcium content and in presence of growth factors, most commonly a combination of epidermal growth factor (EGF) and insulin (Shevchenko et al., 2010 J R Soc Interface 43, 229-258).

In particular, it has been shown that HFSCs that were initially FACS-purified from a freshly isolated mixture of epidermis-derived cells and afterwards grown using such culture conditions known for primary epidermal keratinocytes and also HF-derived keratinocytes comprising epidermis-derived SCs, form tight colonies known as holoclones (Barrandon & Green, 1987, Cell 50: 1131-1137), that contained HFSCs (Blanpain et al., 2004, Cell 118: 635-648, Kandyba et al., 2013, Proc Natl Acad Sci USA, 110: 1351-1356). Such holoclones could be clonally passaged up to 10 (Blanpain et al., 2004, Cell 118: 635-648) and 20 (Kandyba et al., 2013, Proc Natl Acad Sci USA, 110: 1351-1356) times while preserving their typical morphology. Upon passaging, however, the capacity of holoclones to give rise to higher number of colonies decreases (Blanpain et al., 2004, Cell 118: 635-648), indicating that the HFSCs do not self-renew in this system and therefore the number of HFSC cannot be increased using this culture method. This inability to self-renew and therefore the inability to expand cultured HFSCs are major limitations of this method. Another limitation of this method is the need of employing a heterologous feeder cell layer, most commonly fibroblasts, for culturing. Such feeder cell layers are particularly disadvantageous for a number of downstream applications of cultured cells.

In another study, epidermal SCs from newborn mice were cultured on collagen I and fibronectin-coated 2D-surfaces using newborn fibroblast-conditioned medium, i.e. growth media preincubated with fibroblasts, as growth medium (Reiisi et al, 2010, In vitro Cell Dev. Biol. 46:54-59). However, first due to usage of markers that are not sufficient to clearly distinguish SCs from differentiated epidermal progenitor cells, it is not clear whether this method is suitable for culturing epidermis-derived stem cells. Second, it is unclear if such method efficiently maintains multipotency of such epidermal-derived SCs (Reiisi et al, 2010, In vitro Cell Dev. Biol. 46:54-59). Third, another severe limitation of this methods is the requirement of conditioned media from feeder cells. Such conditioned media is similarly to the alternative heterologous feeder cells (most commonly fibroblasts) also disadvantageous as regarding down-stream analysis of cultured cells, but needed to maintain keratinocytes and epidermal SC growth.

Recently, a culturing system for epidermis-derived stem cells that does not involve disadvantageous heterologous feeder cells or newborn fibroblast-conditioned medium has been described. In this method, FACS-purified murine HFSCs were successfully cultured without feeder cells in a 2D-cell layer in the presence of Wnt3a (Ouji et al, 2015, J Invest Dermatol 135: 1598-1608). However, this method has other limitations. In particular, only a small fraction (about 10%) of CD34⁺α6⁺ HFSCs could be maintained, and this only through continuous FACS-based enrichment and subsequent passaging every 10 days. Thus this method is extremely laborious and does not allow for efficient expansion of the HFSC population in vitro.

Another method for culturing epidermis-derived stem cells, in particular HFSCs involves the reprogramming multipotent keratinocytes from induced pluripotent stem cells (iPS cells) and subsequent maintenance of FACS-purified HFSCs (Bilousova & Roop, 2013, Methods Mol Biol 961: 337-350; Lichti et al., 2008, Nat Protoc 3: 799-810; Oh et al., 2013, J Invest Dermatol 133: e14; Ouji et al, 2015, J Invest Dermatol 135: 1598-1608). In this method, certain committed adult cell types, typically for example a fibroblast, is reprogrammed into a iPS cell by introducing a set of transcription factors (KLF4, c-MYC, OCT4, and SOX2, also known as the core Yamanaka factors). The iPS cells are subsequently differentiated into keratinocytes using retinoic acid and BMP-4 to induce differentiation toward a keratinocyte lineage, which is then followed by the growth of differentiated iPS cells on collagen type I- and collagen type IV-coated dishes to enrich for iPS cell-derived keratinocytes. The main disadvantages of this system are the laborious multistep process, lack of complete purity of keratinocytes (80-90%) and the lack of multipotency, i.e. the cells generated through this method have not been demonstrated to form hair (Bilousova & Roop, 2013, Methods Mol Biol 961). Accordingly, although a variety of culture systems have been described for culturing epidermis-derived stem cells, there is a need for an improved culture system for epidermis-derived stem cells. In particular, no culture system for epidermis-derived stem cells is known that allows specific and targeted enrichment and/or expansion of these cells in vitro.

Thus, the technical problem underlying the present invention is the provision of improved means and methods for ex vivo culturing and/or expansion of epidermis-derived stem cells.

The solution is provided by the embodiments as defined herein below and as characterized in the claims.

The present invention relates to a method for culturing epidermis-derived stem cells comprising the step of culturing epidermis-derived stem cells in the presence of a three-dimensional extracellular matrix (3D-ECM) and a basal cell culture medium, said cell culture medium comprising:
Epidermal Growth Factor (EGF); and/or
a Vascular Endothelial Growth Factor (VEGF); and/or
a Fibroblast Growth Factor (FGF);
and further a ROCK (Rho-kinase) inhibitor.

Surprisingly, the present inventors could demonstrate that the above-mentioned method allows for improved culturing of epidermis-derived stem cells ex vivo while preserving an undifferentiated phenotype, self-renewing capabilities and/or multipotency. In particular, culturing epidermis-derived stem cells (e.g. hair follicle stem cells) in the presence of a 3D-ECM and a basal cell culture medium comprising e.g. a ROCK inhibitor (referred to as “Y-E”, see Figures and Examples) or a EGF and a ROCK inhibitor (referred to as “Y”, see Figures and Examples) improves the survival of epidermis-derived stem cells during culturing compared to 2D culture systems with coated cell culture plates (referred to as “KGM 2D”, see Figures and Examples). An even improved survival and/or even a expansion of epidermis-derived stem cells during culturing could, for example, be achieved by a culturing method according to the present invention that employs a 3D-ECM and a basal cell culture medium that comprises EGF, a FGF and a ROCK inhibitor (referred to as “YF”, see Figures and Examples); EGF, a VEGF and a ROCK inhibitor (referred to as “YV”, see Figures and Examples); or a FGF, a VEGF and a ROCK inhibitor (referred to as “3C-E”, see Figures and Examples). An even further improved survival and/or expansion of epidermis-derived stem cells during culturing could be achieved by a culturing method according to the present invention that employs a 3D-ECM and a basal cell culture medium that comprises EGF, a FGF, a VEGF and a ROCK inhibitor (referred to as “3C”, see Figures and Examples). The method for culturing epidermis-derived stem cells according to the present invention involves culturing of these cells in so-called “3D” cultures that are characterized by the presence of a three-dimensional extracellular matrix (3D-ECM) and a specially adopted cell culture medium that consists of a basal cell culture medium comprising one or more mitogenic growth factor(s) selected from the group consisting of EGF, a VEGF and a FGF; and further comprising a ROCK inhibitor. The provided ECM forms a three-dimensional structure (3D-ECM) in which the cultured epidermis-derived stem cells are embedded. This is achieved by mixing the cells and the ECM components before the ECM components solidify. This is in contrast to known conventional 2D culture systems, in which the cell culture plates are coated with ECM components such as collagen I to form a thin, adhesive layer, and the cells are seeded on these coated plates after the ECM components have adhered to the surface of the tissue culture plastic. As shown in the Figures and the Examples culturing epidermis-derived stem cells in the presence of a 3D-ECM (referred to as “3D” culturing) is crucial for the method of culturing epidermis-derived stem cells according to the present invention. Furthermore, the inventors could demonstrate that different types of 3D-ECMs such as 3D-ECMs generated with commercially available ECMs such as basement membrane extracts (Matrigel, BME) or gels comprising a collagen (e.g. collagen I) or 3D-ECM gels comprising a collagen (e.g. collagen I) and a laminin (e.g. laminin-332; laminin-511; or laminin-332 and laminin-511) can be employed in the method for culturing epidermis-derived stem cells according to the present invention (see Figures and Examples). As regarding the cell culture medium, it has been surprisingly found that a basal cell culture medium for human and/or animal cells, which comprises the above mentioned mitogenic growth factor(s) and a ROCK inhibitor, is particularly advantageous for culturing epidermis-derived stem cells, in particular in the presence of 3D-ECM (see Figures and Examples).

In the context of the present invention it was surprisingly found that only by combining of culturing epidermis-derived stem cells such as HFSCs in presence of 3D-ECM (“3D” culturing conditions) with culturing in the presence of a basal cell culture medium comprising the above mentioned mitogenic growth factor(s) and ROCK inhibitor(s) can maintain epidermis-derived stem cells with the corresponding cellular markers efficiently (see Figures and Examples). By contrast, standard “2D” keratinocyte culturing methods known in the art resulted in a rapid depletion of epidermis-derived stem cells, even if employing basal cell culture medium according to the present invention (see Figures and Examples).

As mentioned above, the method for culturing epidermis-derived stem cells preferably comprises the step of culturing epidermis-derived stem cells in the presence of a three-dimensional extracellular matrix (3D-ECM) and a basal cell culture medium comprising:
Epidermal Growth Factor (EGF); and
a Vascular Endothelial Growth Factor (VEGF); and
a Fibroblast Growth Factor (FGF);
and further a ROCK (Rho-kinase) inhibitor.

Such culturing conditions as regarding the provision of a three-dimensional extracellular matrix (3D-ECM) and the basal cell culture medium mentioned above are referred to as “3C”, “3C conditions” or “3C culturing conditions” elsewhere herein. The term “3C medium” refers to the cell culture medium composition of these conditions. The 3C culturing conditions allow for culturing of epidermis-derived stem cells in the sense of maintenance/survival of epidermis-derived stem cells and additionally also trigger expansion/growth and/or enrichment of epidermis-derived stem cells in cell mixtures. For instance, in cultured cell mixtures comprising epidermis-derived stem cells, such 3C conditions result in an increase in the proportion of epidermis-derived stem cells with a simultaneous increase in the absolute stem cell numbers in the culture. As shown e.g. in Figures and Examples, the proportion of HFSCs in bulk keratinocyte cultures could be increased from about 5.6% to more than 40% in only 14 days of culturing under 3C conditions. In addition, the absolute number of epidermis-derived stem cells increased approximately 5-fold in only 14 days. Similarly, also cultivation of tumour cells that have been isolated from papillomas resulted in an increase proportion of comprised epidermis-derived cancer stem cells (see Figures and Examples).

The present invention also provides for a method for expanding epidermis-derived stem cells comprising the step of culturing epidermis-derived stem cells in the presence of a three-dimensional extracellular matrix (3D-ECM) and a basal cell culture medium comprising Epidermal Growth Factor (EGF), a Vascular Endothelial Growth Factor (VEGF), a Fibroblast Growth Factor (FGF) and a Rho-kinase (ROCK) inhibitor.

In the context of the present invention, it was surprisingly found that with the above-mentioned method epidermis-derived stem cells can be expanded in vitro. By contrast, other methods known in the art can only achieve maintenance/survival of epidermis-derived stem cells to a certain degree but not expand these cells. Thus, the method surprisingly allows for efficiently amplifying epidermis-derived stem cells in vitro.

In the context of the method for expanding epidermis-derived stem cells the term “expanding” means that the number of cells, e.g. of a specific cell type (e.g. epidermis-derived stem cells) increases during the time of in vitro culturing by mitotic cell division. In principle, expansion of the cultivated cells during culturing can be unlimited. Preferably, expansion means to increase the number of epidermis-derived stem cells by at least 1.5-fold, 2-fold, 5-fold, 10-fold, 20-fold, 50-fold, 100-fold or 200-fold during the time of culturing. Culturing in this context relates to maintaining cells in vitro/ex vivo.

The present invention provides an improved method for culturing epidermis-derived stem cells which allows to expand epidermis-derived stem cells in vitro in an in principle unlimited manner. Also provided is a method that allows expansion of epidermis-derived stem cells. Thus, epidermis-derived stem cells can be expanded in large quantities amenable for high-throughput applications.

In particular, the present inventors surprisingly found that with the method according to the present invention, epidermis-derived SCs such as HFSCs or CSCs (originating from epidermis-derived papilloma or carcinoma) can be maintained without losing the surface proteins that are characteristic markers for their stem cell identity. Furthermore, by using the culturing method according to the present invention, the cultured epidermis-derived SCs maintain their multipotency and/or self-renewing capability. Thus, the epidermis-derived stem cells remain their capability of regenerating epidermal and/or hair follicle tissue (see Figures and Examples). Accordingly, the epidermis-derived stem cells cultured by the method of the present invention can give rise to intact epidermal tissue and/or hair follicles and thus can be used, for example, in tissue engineering and or tissue transplantation. Surprisingly, HFSCs cultured with the method according to the invention can give rise to hair growth even more efficiently than freshly isolated keratinocytes comprising HFSCs (see Figures and Examples).

Other methods for ex vivo culturing of epidermis-derived stem cells, such as HFSCs, known in the art involve repetitive rounds of enriching for stem cells which maintained the respective stem cell surface markers, because otherwise those cells would be depleted during culturing. Thus, the method for culturing epidermis-derived stem cells according to the present invention is much less time and resource consuming, i.e. it is more time- and/or resource-efficient. The present invention therefore provides an improved, highly reliable and easy to handle method for culturing epidermis-derived stem cells. Also provided is a method that allows expansion of epidermis-derived stem cells.

A particular further advantage of the method for culturing epidermis-derived stem cells according to the present invention is that the method does not necessarily involve feeder cells and is therefore more robust, more reproducible, more easy to manipulate and more amenable to scale-up applications than the culturing methods known in the art that make use of feeder cells or conditioned medium from feeder cells. Such a feeder-free culturing system is more precisely defined and can, for example, be particularly advantageous for studying basic cellular processes such as SC fate determination, receptor-ligand responses and cell signaling.

A particular advantage of the method for culturing epidermis-derived stem cells according to the present invention is that this method unlike other available culturing methods can not only be applied to previously purified epidermis-derived stem cells but can also easily be established from bulk keratinocytes comprising said epidermis-derived stem cells (e.g. see Figures and Examples). Thereby the method provides the possibility to avoid the laborious FACS-sorting needed to purify particular epidermis-derived stem cells prior to culturing that is an essential step of most culturing methods described in the prior art.

Furthermore, the method for culturing of epidermis-derived SCs does not require any steps of FACS-purification of epidermis-derived stem cells during culturing. This is a step that some known culturing methods employ in order to avoid the depletion of epidermis-derived SCs from the culture. The method according to the present invention establishes, however, a stable equilibrium between epidermis-derived SCs and non-SCs without any additional FACS purification of epidermis-derived stem cells during culturing.

The method for culturing epidermis-derived stem cells according to the present invention also provides the advantage of preserving the karyotypic integrity, the multipotency and the self-renewal capability of epidermis-derived stem cells such as e.g. HFSCs, even after long-term culturing. Long-term in this context means at least 360 days, at least 250 days, at least 100 days, at least 50 days, at least 20 days, at least 14 days, at least 10 days, at least 8 days, at least 5 days, at least 3 days or at least 2 days.

Another advantage compared to methods for culturing epidermis-derived stem cells currently known in the art is that the epidermis-derived stem cells resulting from the method according to the present invention can be freeze-thawn and therefore stored during culturing for later applications. Notably, a freeze-thaw cycle does not result in an evident loss of epidermis-derived stem cells and their multipotency (see Figures and Examples). The increased freeze-thaw resistance is in particular of commercial relevance, as it, for example allows for shipping of the epidermis-derived stem cells. A further advantage resulting from the increased freeze-thaw resistance is that it allows for long-term storage of epidermis-derived stem cells after which these cells can be re-cultured with a method according to the present invention. None of the previously reported methods for culturing epidermis-derived SCs has reportedly permitted freezing and subsequent thawing and repropagation of SCs without loss of their SC properties.

Surprisingly, the present inventors identified that the method for culturing epidermis-derived stem cells according to the present invention always results in a mixture of epidermis-derived stem cells and differentiated progeny cells independent of the starting material (see Figures and Examples). In other words even if isolated epidermis-derived stem cells are subjected to the culturing method of the present invention the cultivation will result in a mixture of cells comprising epidermis-derived stem cells and differentiated progeny cells (see Figures and Examples). Notably, a stable equilibrium of stem cells and differentiated cells is reached within only 12 days and is stably maintained. The resulting cell mixture is particularly advantageous to efficiently maintain and/or expand/proliferate the epidermis-derived stem cells in vitro.

Importantly, for transplantation experiments typically freshly isolated keratinocyte cell mixtures are used. These freshly isolated mixtures, however, comprise a much lower proportion of epidermis-derived stem cells and actually also are less efficient than epidermis-derived stem cells comprised in a cell mixture that arises from the culturing method of the present invention (e.g. under 3C conditions) as, for example shown in FIG. 16. Accordingly, the method according to the present invention can even provide more potent cell mixtures for medical or cosmetic transplantation.

Moreover, if an application of epidermis-derived stem cells requires to produce pure/isolated epidermis-derived stem cells, the method according to the present invention may further comprise a step of isolating said epidermis-derived stem cells; i.e. isolating the cells e.g. by FACS-sorting based on the cellular markers of the respective epidermis-derived stem cells as further specified herein elsewhere.

In one aspect the method for culturing epidermis-derived stem cells according to the present invention employs an ECM, in particular a 3D-ECM, and a basal cell culture medium comprising EGF, a VEGF, a FGF and a ROCK inhibitor/antagonist, wherein said basal cell culture medium further comprises a Sonic Hedgehog (SHH) inhibitor/antagonist.

As shown in the Figures and Examples, further addition of a SHH inhibitor cyclopamine increased the proportion of HFSCs (as example for epidermis-derived stem cells) in the cultured cell mixture up to 93.6%. By contrast, 3C culturing conditions typically resulted in a cell mixture comprising around 50% of HFSCs. Accordingly, the further addition of SHH inhibitor to the culture medium provides the possibility to gain cell mixtures which have an even higher content of epidermis-derived stem cells. Actually, even nearly pure epidermis-derived SC cultures can be gained with the method according to the present invention. In this regard, according to one aspect of the present invention, the epidermis-derived stem cells may also be first cultured under 3C culturing conditions and after some passages under 3C culturing conditions further comprising a SHH inhibitor. Notably, methods known in the art for culturing epidermis-derived SCs, even if starting from nearly pure, FACS-purified epidermis-derived stem cells result in cell mixtures with much lower SC content. In particular, also organoid cultures that have been described for epithelial stem cells other than epidermis-derived SCs, are not capable of generating cultures with such a high SC proportion.

The invention also relates to a method that allows for ex vivo de novo conversion of differentiated epidermal cells (that lack epidermis-derived SCs) into epidermis-derived SCs with undifferentiated phenotype and self-maintenance capabilities. The epidermis-derived stem cells generated with this method can, for example, also be subsequently used in the method for culturing epidermis-derived SCs. In principle, the method for ex vivo de novo conversion of differentiated epidermal cells can be performed as described herein for the method for culturing epidermis-derived stem cells. Preferably, also here 3C culturing conditions are employed. Importantly, de novo generation of epidermis-derived stem cells can subsequently also be cultured (also in long-term) by the method for culturing epidermis-derived stem cells according to the present invention. As described above for the method of culturing epidermis-derived stem cells, the method for de novo generation of epidermis-derived stem cells also results in a cell mixture, which comprises epidermis-derived stem cells and differentiated cells (see Figures and Examples). Similarly, after around 12 days of culturing an equilibrium between epidermis-derived stem cells and differentiated cells (e.g. with about 40-60% epidermis-derived stem cells; see Figures and Examples) is reached. In the Figures and Examples, the ex vivo de novo conversion of HFSCs from non-HFSCs could exemplary be demonstrated.

The method for ex vivo de novo conversion of differentiated epidermal cells according to the present invention is particularly beneficial if an organism/individual that lacks certain epidermis-derived stem cells. In such scenarios, epidermis-derived stem cells can be de novo generated in vitro from differentiated cells and used for the treatment of such diseases/scenarios. For example, such a treatment can be envisaged in the treatment of Alopecia, a disease where patients lose hair due to destruction of HFs and/or loss of HFSCs, thereby resulting in inability to regenerate hair.

In general, epidermis-derived stem cells such as HFSCs originating from a method of culturing and/or a method for de novo generation of HFSCs can, for example be applied in the field of regenerative medicine, in particular, for example, for treatment of skin diseases, skin injuries and/or alopecia.

As mentioned above, the present invention relates to a method for culturing epidermis-derived SCs comprising the step of culturing epidermis-derived SCs in the presence of a three-dimensional extracellular matrix (3D-ECM) and a basal cell culture medium comprising:
Epidermal Growth Factor (EGF); and/or
a Vascular Endothelial Growth Factor (VEGF); and/or
a Fibroblast Growth Factor (FGF);
and further a ROCK (Rho-kinase) inhibitor.

Moreover, the present invention also relates to a method for culturing epidermis-derived stem cells comprising the step of culturing epidermis-derived stem cells in the presence of a three-dimensional extracellular matrix (3D-ECM) and a basal cell culture medium comprising a ROCK (Rho-kinase) inhibitor. Already the presence of only a ROCK inhibitor improves the survival of epidermis-derived stem cells (preferably HFSCs) compared to standard 2D culturing methods.

The present invention further relates to a method for culturing epidermis-derived stem cells comprising the step of culturing epidermis-derived stem cells in the presence of a three-dimensional extracellular matrix (3D-ECM) and a basal cell culture medium comprising:
Epidermal Growth Factor (EGF); and
a Vascular Endothelial Growth Factor (VEGF); and/or
a Fibroblast Growth Factor (FGF);
and further a ROCK (Rho-kinase) inhibitor.

Similarly, the present invention relates to a method for culturing epidermis-derived stem cells comprising the step of culturing epidermis-derived stem cells in the presence of a three-dimensional extracellular matrix (3D-ECM) and a basal cell culture medium comprising:
Epidermal Growth Factor (EGF); and
a Vascular Endothelial Growth Factor (VEGF); and
a Fibroblast Growth Factor (FGF);
and further a ROCK (Rho-kinase) inhibitor.

In the context of the present invention the term “epidermis-derived stem cell”, or as interchangeably also referred to as “epidermal stem cell” herein, means a stem cell (SC) that is contained in the epidermis/epidermal tissue of mammals and/or can be derived from the epidermis/epidermal tissue of mammals (e.g. by method known in the art to isolate epidermal cells as described further below), including also the epidermis/epidermal tissue of mammals that comprises papilloma or carcinoma. In particular, an epidermis-derived stem cell according to the present invention is characterized by high expression of integrins α6 and/or β1. Specific antibodies that recognize these cell surface proteins can be used to identify these cells (e.g. by FACS) and/or to isolate them from the tissue using for example FACS purification.

Examples of antibodies that can be used in FACS analysis and FACS purification are anti-CD34 (clone RAM34, for example from eBioscience or BD Biosciences), anti-α6 integrin (clone GoH3, for example from eBioscience, Abcam, Novus Biologicals), anti-CD200 (clone OX90, for example from eBioscience, Abcam, Thermo Fischer), anti-EpCAM (clone G8.8, for example from eBioscience, Abcam, Thermo Fischer), anti-CD140a (clone APA5, for example from eBioscience, Abcam, Thermo Fischer); anti-CD45 (clone 30-F11, for example from eBioscience, Novus Biologicals, Thermo Fischer), anti-CD31 (clone MEC13.3, for example from eBioscience, Novus Biologicals).

To label cells with specific antibodies for FACS analysis or purification for example the following protocol can be used. Single cell suspensions are incubated for example with the above-mentioned antibodies for 30 min at +4° C. After two washes with FACS Buffer (2% FCS, 2 mM EDTA in phosphate-buffered saline) cells are analyzed in a flow cytometer such as the FACSCanto II (BD Biosciences) or purified in a flow cytometer such as FACSAria II or a FACSAria Fusion (both from BD Biosciences). Within the flow cytometer sample cells are passed through a narrow channel one at a time, and light is used to illuminate the cells in the channel. A series of sensors detect the types of light that are refracted or emitted from the cells. Data acquired by the sensors is compiled and integrated to build a dataset where the measured intensities of each cell are represented allowing quantification of staining intensities or purification of cell populations according to specific staining intensities.

Expression of cell surface markers is analyzed on live cells after exclusion of cell doublets and dead cells using DAPI (2-(4-amidinophenyl)-1H-indole-6-carboxamidine), 7AAD (eBioscience) or Fixable Viability Dye eFluor405 (eBioscience).

In particular, in the context of the present invention epidermis-derived stem cells/epidermal SCs are, for example, mammalian HFSCs, mammalian interfollicular epidermis SCs, and mammalian cancer stem cells (CSCs), wherein said CSCs originate from the skin and/or epidermis. In the context of the present invention, CSCs originating from the skin and/or epidermis are, for example, SCs that reside in a skin papilloma and/or carcinoma.

Furthermore, the terms “epidermis-derived stem cell” or “epidermal stem cell” according to the present invention also includes a stem cell with high expression of integrins α6 and β1 generated in vitro from stem cells, preferably induced pluripotent stem cells (iPS cells).

In this case iPS cells are differentiated into keratinocytes using retinoic acid and bone-morphogenetic protein-4 to induce differentiation toward a keratinocyte lineage, which is then followed by the growth of differentiated iPS cells on collagen type I- and collagen type IV-coated dishes to enrich for iPS cell-derived keratinocytes as described in Kogut et al, 2014, Methods Mol Biol, 1195: 1-12.

The epidermis comprises multiple, independent epidermis-derived stem cell populations that exist in various specialized microenvironments termed niches within the epidermis and the hair follicle. One of the most important SCs reside in the hair follicle and are termed hair follicle stem cells (HFSCs). HFSCs have the capacity to differentiate along multiple lineages and give rise to hair follicles, interfollicular epidermis and sebaceous glands. HFSCs can be identified by the expression of high levels of α6 integrins and/or β1 integrins and CD34 on their cell surface. In addition, these cells are marked by Sox9, K15, Nfatc1, Lhx2, Lgr5, and Lgr6 expression. Of these, α6 integrins, CD34, Lgr5, and Lgr6 are cell surface markers that particularly can be used to isolate these cells, e.g. by FACS as described above. In this context for example the following antibodies can be employed: anti-CD34 (clone RAM34, eBioscience, BD Biosciences), anti-α6 Integrin (clone GoH3, eBioscience, Abcam, Novus Biologicals).

Gene profiling of bulge hair follicle stem cells suggests that they exist in two states. Quiescent stem cells express high levels of cell cycle inhibitors and downregulate cell cycle promoters, thus maintaining quiescence. These quiescent stem cells can be primed, leading to their translocation downwards into the hair germ of the HF. The primed stem cells give rise to the activated transit amplifying cells of the hair matrix, at the onset of the anagen growth phase.

Apart from the bulge, other hair follicle stem cell niches are the junctional/infundibulum zone that expresses Lrig1 and the isthmus that expresses Lgr6 and Plet1 (also called MTS24). All of these hair follicle stem cell populations seem to be distinct from CD34⁺ bulge SCs. Lgr6⁺ and Lrig1 SCs have been shown to be multipotent during development and in skin reconstitution experiments, respectively. However, during normal homeostasis Lrig1⁺ stem cells have been shown to be bipotent and only contribute to the regeneration of interfollicular epidermis and the sebaceous gland.

The interfollicular epidermis also contains of stem or progenitor cells. These keratinocytes, when plated at low clonal density, can give rise to highly proliferative colonies (holoclones), aborted colonies (paraclones) and relatively small heterogeneous colonies (meroclones). The fact that the in vitro progeny of a single cell from such holoclone can give rise to a functional self-renewable epidermis in vivo, suggests that these cells are indeed stem cells or progenitors. Also when grafted on the body of burn patients, interfollicular epidermal cells can produce viable and functional epidermal sheets. Like hair follicle stem cells, the stem cells of the interfollicular epidermis express high levels of α6 and/or β1 integrins.

The mammalian epidermis/epidermal tissue is composed of a pilosebaceous unit that consists of the hair follicle (HF), the sebaceous gland and the surrounding interfollicular epidermis. Each pilosebaceous unit contains multiple distinct SC populations that fuel the constant renewal of the interfollicular epidermis and HFs during postnatal tissue homeostasis and regeneration.

The epidermis, comprising of keratinocytes, is a multilayered stratified epithelium consisting of a basal, undifferentiated layer of stem cells and several suprabasal, differentiated cell layers. The stem cells of the basal layer are mitotically active and characterized by the expression of certain intermediate filament proteins called keratins.

As basal to suprabasal fate switch occurs, cells switch off the expression of integrins with a concomitant switch of keratin expression. Suprabasal cells contribute to three distinct layers of a stratified epithelium. The first layer is the spinous layer where the cells generate even more robust network of intermediate filament proteins. As cells move upwards, they form the second granular layer, named according to the presence of additional structural proteins deposited below the plasma membrane making these cells appear granular. Loricrin and Filaggrin are produced and mark the granular layer. During subsequent stages of differentiation specialized lipids are extruded out of the cell from intracellular lamellar granules in the extracellular space, thus sealing the skin surface with an indestructible fibrous mass of keratins and sandwiched with lipids in the outermost stratum corneum layer. This layer is eventually shed from the body. Mammalian epidermis is known to renew every two weeks making the skin one of the most active self-renewing organs of the body. Tight regulation of stem cell self-renewal and differentiation is of central importance to this process.

The epidermis also comprises hair follicles, organs that are generated from the interfollicular epidermis and specialize to generate hair. Hair follicle morphogenesis is initiated by an inductive bi-directional signaling crosstalk between keratinocytes that eventually form the HF and a specialized population of dermal fibroblasts that constitute the dermal papilla. This crosstalk that involves multiple signaling pathways ultimately results in the down growth of the hair follicle through a combination of cell migration and proliferation. After morphogenesis during postnatal life, the hair coat is constantly renewed. This occurs by renewing the lower portion of the hair follicle in cycling phases of growth (anagen), regression (catagen), and rest (telogen). The bulge is the niche for quiescent SCs that during anagen are activated to leave the niche to fuel the growing hair follicle. Tight regulation of stem cell quiescence and activation is required to maintain a stable pool of stem cells enabling HF cycling throughout the lifetime of an individual.

Cells that are contained in the epidermis and/or can be derived from the epidermis are termed “epidermis-derived” in the context of the present invention.

In the context of the present invention the term “stem cells” relates to undifferentiated biological cells that can differentiate into specialized cells and can divide (through mitosis) to produce more stem cells (a process referred to as self-renewal). They are found in multicellular organisms. In mammals, there are two broad types of stem cells: embryonic stem cells, which are isolated from the inner cell mass of blastocysts, and adult somatic stem cells (SCs), which are found in various tissues. In the context of the present invention the term stem cells refers to the group of adult somatic stem cells. Adult somatic stem cells maintain organ form and function. The characteristics of stem cells vary according to tissue of origin, but they share the following properties: an undifferentiated phenotype; potency to differentiate toward all lineages of the tissue of origin (multipotency); capacity to self-renew; capacity to regenerate the tissue upon injury. The homeostatic and reparative functions of stem cells require that their behavior is adjusted to the needs of the tissue. Hence, stem cells must be able to gain inputs on the state of the tissue and organism and transform these inputs into fate decisions. The stem cells reside in specific anatomical locations termed niches that integrate information from the local tissue environment as well as from systemic signals. To be able to execute these functions, niches are complex system composing of multiple cell types and a specialized extracellular matrix.

Stem cells are typically identified by one cellular marker or a combination of cellular markers. These markers or marker combinations are in most cases proteins, which are expressed in stem cells, in particular on their cell surface. Typically one or several of such marker proteins (combinations of markers), which can e.g. be detected by FACS/flow cytometry analysis as described further below, can clearly identify a stem cell and/or stem cell family/class. The markers typically differ between different types of stem cells. As mentioned above, epidermis-derived stem cells are in particular characterized by high expression of integrins α6 and β1.

In the context of the present invention in principle any epidermis-derived stem cell can be employed. Preferably, a epidermis-derived stem cell selected from the group consisting of mammalian hair follicle stem cells (HFSCs), mammalian interfollicular epidermal stem cells, and mammalian CSCs wherein said CSCs originate from the epidermis (in particular the epidermis of papilloma- or carcinoma-comprising skin) are, for example epidermis-derived stem cells known in the art. Preferably, in the context of the present invention epidermis-derived stem cells/epidermal stem cells are selected from the group consisting of HFSCs and epidermis-derived cancer stem cells. Said epidermal-derived cancer stem cells are preferably selected from the group consisting of papilloma-derived and carcinoma-derived stem cells. Accordingly, preferably the method for culturing epidermis-derived stem cells is a method for culturing epidermis-derived stem cells selected from the group consisting of hair follicle stem cells and epidermis-derived cancer stem cells. Most preferably, the method for culturing epidermis-derived stem cells is a method for culturing hair follicle stem cells. Furthermore the epidermis-derived stem cells employed in the context of the present invention are preferably of human or murine origin, most preferably human origin.

Preferably, epidermis-derived stem cells cultured with the method for culturing epidermis-derived stem cells according to the present invention are comprised in an isolated tissue fragment; are isolated/pure, i.e. the cells cultured do not comprise significant amounts of other cell types; or preferably are comprised in a mixture of cell types, which further comprises at least one differentiated epidermal cell type (e.g. such mixture can be bulk keratinocytes/bulk skin cell mixtures). The term “not comprise significant amounts” in the context of isolated/pure cells means less than 1%, 2%, 3%, 5% or 10% of cells other than epidermis-derived stem cells. The remaining amount of other epidermal cell types can, for example, be determined by FACS-analysis. Such FACS-analysis, as described above, allows to determine the proportion of epidermis-derived stem cells based on the markers mentioned above for epidermis-derived stem cells in general and certain types epidermis-derived stem cells. Respective antibodies for the FACS-analysis are preferably selected depending on the type of epidermis-derived stem cells. The amount of other epidermal stem cell can be calculated by subtracting the number of epidermis-derived stem cells from 100%. If epidermis-derived stem cells are cultured in said mixture of cell types, such mixture of cell types preferably comprises isolated keratinocytes, epidermal papilloma cells or epidermal carcinoma cells. Keratinocytes, such as bulk keratinocytes preferably comprise one or more cell types of the epidermis. These are among others: epidermal progenitor cells, transit amplifying cells, outer root sheath cells, hair germ cells, hair matrix cells and keratinocytes.

Most preferably, in the method according to the present invention cell mixtures comprising epidermis-derived stem cells are crude single-cell suspension derivable from tissue biopsies as described further below. As mentioned herein elsewhere the method according to the present invention has the particular advantage of being able to start from such crude keratinocyte samples and therefore does not necessarily require further purification (e.g. by FACS) of epidermis-derived stem cells.

The preferred number of cells used to initiate a culture employing isolated/pure epidermis-derived stem cells or a cell mixture is between 1×10⁴ and 3×10⁵ cells or at least 2×10⁴ and not higher than 3×10⁵ cells. A preferred number is at least 1×10⁴, 2×10⁴, 3×10⁴, 4×10⁴, 5×10⁴, 6×10⁴, 7×10⁴, 8×10⁴, 9×10⁴, 1×10⁵, cells and not higher than 2×10⁵, 3×10⁵, 4×10⁵, 5×10⁵ cells. A more preferred number is at least 7×10⁴ and not higher than 9×10⁴ cells. An even more preferred number is about 8×10⁴ cells or 8×10⁴ cells. Preferably the initial cells comprise at least 1%, preferably at least 2%, most preferably at least 4% epidermis-derived stem cells.

In the context of tissue fragments, preferably tissue fragments comprising epidermis-derived stem cells, wherein said tissue is skin are cultured with the method of the present invention. Skin fragments can be isolated by protocols that are known to the person skilled in the art. For example, skin fragments may be isolated by obtaining a punch biopsy of the skin or excising a piece of skin using a surgical scalpel.

Cell mixtures/populations comprising epidermis-derived stem cells can be isolated/gained by protocols that are known to the person skilled in the art. For example, skin fragments may be isolated from a tissue biopsy using trypsin or other proteolytic enzymes to degrade the basement membrane, thereby separating the epidermis from the underlying connective tissue cells in a form of a single-cell suspension. For example, skin biopsies can be obtained by incubating skin biopsies in 0.8% Trypsin (for example from Gibco) for 50 min at 37° C. after which the epidermis can be mechanically separated from the dermis by scraping. Single cells can then be separated by passing the epidermis tissue through 70 or 45 μm cell strainers (for example from BD Biosciences). If desired, epidermis-derived stem cells or only particular epidermis-derived stem cell types (e.g. HFSCs) can be further purified from the cell suspension using antibodies against the specific cell surface antigens and FACS sorting as described above.

In particular, cell mixtures/populations containing interfollicular epidermal stem cells can be obtained by protocols that are known to the skilled person. For example, cells can be isolated by incubation of isolated tissue with proteases such as trypsin that release cells from the basement membrane. After separation, the keratinocyte cell layer along with the hair follicle cells is mechanically separated from the dermis and minced. This is followed by extraction of single cells using, for example, filtration and/or centrifugation steps. Other proteolytic enzymes, such as dispase I, can be used instead of or in combination with trypsin.

Hair follicle stem cells can also be isolated/separated/purified from other cell types prior to culturing. Methods to specifically separate hair follicle stem cells from other skin cells are known in the art. A preferred method is based on the fact that hair follicle stem cells express CD34 and α6 integrin on their surface. A preferred method comprises preparing a cell suspension from the epithelial tissue, contacting the cell suspension with CD34/α6 integrin binding compound, removing the non-bound, excess of the CD34 and α6 integrin binding compound by washing, and isolating the stem cells that bind the binding compound. In another alternative method, stem cell surface markers are Lrig1, Lgr5, and Lgr6. In addition, it will be clear to a skilled person that stem cells can further be isolated from genetically engineered mice where promoters of known stem cell markers such as Lgr5, Lgr6, Sox9, Keratin15, Lrig5 control the expression of a reporter protein such as, for example, GFP, YFP, RFP, and LacZ.

Fluorescence-activated cell sorting (FACS) and magnetic bead-based cell separation make use of labeled-binding compounds e.g. antibodies that specifically recognize cell surface proteins. After labeling epidermal-derived cell mixtures with said binding compounds stem cells can be isolated with high purity (up to 99.9%) from the non-stem cells, which do not bind said binding compounds. FACS makes use of lasers to excite fluorophores attached to the binding compounds and then specifically selects/purifies cells that are bound by said binding compounds. Magnetic bead-based cell separation also isolates cells that specifically bind binding compounds with the help of antibodies attached to magnetic beads. These antibodies recognize the fluorophores or labels (e.g. biotin) attached to the binding compounds. Subsequently, the magnetic beads and any binding compounds and cells attached to them are isolated from solution with a magnet and eluted into a buffer solution. Magnetic bead-based cell separation of HFSCs with multiple binding compounds needs to be carried sequentially.

Epidermis-derived cancer stem cells (CSCs), derived from for example papilloma or carcinoma cells, can be isolated from tumor tissue by methods known in the art. In particular, as for example described for the isolation of cancer stem cells from epidermal papilloma or carcinoma, methods comprising the use of dissociating agents such as dispase I, collagenase, trypsin and EDTA and/or mechanical disruption may be employed to obtain single cell suspensions containing CSCs. For example cell suspensions can be generated from tumor biopsies by first mechanically mincing them using a surgical scalpel and subsequently incubating them in 0.25% collagenase (for example from Sigma), 62.5 U/mL DNaseI (Roche) in Hank's Balanced Salt Solution, (for example from Gibco) for 60 min with gentle agitation at 37° C. Single cell suspensions can be obtained by passing the cell suspension through a 45 μm cell strainer (for example from BD Biosciences).

CSCs can be further purified from the single cell suspension by methods known in the art. These methods include but are not limited to FACS and magnetic bead separation, as describe above for other epidermis-derived stem cells. In particular, for separating CSCs from papilloma or/carcinoma the cell surface markers CD34 and α6 integrin can be employed as some CSCs express CD34 and α6 and/or β1 integrin. These cells should be further express EpCAM and be negative for CD31, CD140a and CD45.

In the case of CSCs from papilloma and/or carcinoma, however, also additional cell surface markers others than CD34 and α6 integrin are employed and are known in the art. For example a surface marker set to isolate CSCs from papilloma or/carcinoma contains at least the following markers: CD140a, CD31, CD45, EpCAM, CD34 and α6 integrin, wherein CSCs are EpCAM-, CD34- and α6 integrin-positive and negative for CD140a, CD31 and CD45. Cells expressing these markers can be isolated from the papilloma or carcinoma using CD140a-, CD31-, CD45-, EpCAM-, CD34- and α6-binding compounds, followed by magnetic bead separation and/or FACS as described for other epidermis-derived stem cells. Another example of a surface marker set to isolate CSCs from papilloma or/carcinoma contains at least the following markers: CD140a, CD31, CD45, EpCAM, CD34, β1 integrin and α6 integrin, wherein CSCs are EpCAM-, CD34-, β1 integrin- and α6 integrin-positive and negative for CD140a, CD31 and CD45. These cells can be isolated as describe for the first set of markers above.

In the context of the present invention the term “culture” or “culturing”, which is also referred to as “cultivating” elsewhere herein, particularly relates to maintaining and/or preferably expanding/growing cells of multicellular organisms, in particular mammals ex vivo. Said culturing is a method which is well known in the art and is, for example described in Phelan, 2007, Curr Protoc Cell Biol, 36: 1.1.1-1.1.18. Typically culturing involves the provision of cell culture medium to the cells (which is described further below) and exchanging this cell culture medium in regular time frames by fresh cell culture medium. Cultures are preferably incubated/cultured at 34-37° C., preferably 37° C. Furthermore, cells are preferably cultured in an atmosphere comprising between 5% and 10%, preferably 5% CO₂. The cell culture medium is preferably exchanged every 2, 3, 4, 5, 6, or 7 days. Most preferably every 2 days. During culturing cells are preferably also regularly removed from the cultivation vessel and all or some of these cells are transferred again in the same or a fresh cultivation vessel to allow for further maintenance and/or expansion/growth of the cells. This is referred to as cell passaging and after each round of passage the cells are typically referred to a corresponding passage, i.e. for example cells that have been passaged twice are referred to as passage two cells. As discussed below said passaging in the context of the presence invention typically also comprises the isolation of the cultured cells from the 3D-ECM. Passaging of cells may, for example, be performed every 2, 4, 6, 8, 10, 12, 14, 16, 18, 20, 22, 24, 26, 28, 30 days. Most preferably every 14 days.

In the context of the method according to the present invention, epidermis-derived SCs are cultured in the presence of a three-dimensional extracellular matrix (3D-ECM). In the context of the present invention a “three-dimensional extracellular matrix (3D-ECM)” refers to a three-dimensionally shaped microenvironment or structure comprising at least one ECM component. The 3D-ECM microenvironment is preferably constituted in a manner that cells, preferably single cells can be/are embedded in a way that they are surrounded by 3D-ECM components. Accordingly, in other words a 3D-ECM is preferably an ECM that surrounds the embedded cells. This can, for example be achieved by mixing cells and liquid ECM components before such ECM components solidify and build a gel-like ECM. The purpose of forming a 3D-ECM is to mimic in vitro the conditions in vivo, in which cells are typically embedded inside an extracellular matrix.

“In the presence of a 3D-ECM” means that the cells are embedded within such 3D-ECM. Accordingly, a preferred method for culturing epidermis-derived stem cells is further a method, wherein said epidermis-derived stem cells are embedded in said 3D-ECM. In other words, ECM or one or more ECM component(s) is/are provided and mixed with the cells intended to be embedded (e.g. epidermis-derived stem cells) before the ECM has solidified. In this context preferably, a 3D shape is generated by forming a droplet as described below and/or by using a structured shape in which the mixture is embedded. A method for culturing and/or generating cells according to the present invention employed “in presence of a 3D-ECM” may also be expressed as a method employed in the presence of ECM, wherein said cells are embedded in the ECM. Optionally this alternative expression for “in presence of a 3D-ECM” may further comprise: “wherein the ECM forms a three-dimensional structure”.

For instance, cells can be embedded within an extracellular matrix, or in other words a 3D-ECM (comprising cells) may be generated by methods known in the art as described for example in Haycock, 2011, Methods Mol Biol 695: 1-15. Preferably, for example, according to the following protocol: epidermis-derived stem cells (e.g. gained by one of the methods described above) are harvested by centrifugation and resuspended in a buffer solution such as a keratinocyte growth medium (e.g. MEM Spinner's modification (Sigma) containing 8% calcium-depleted fetal calf serum) containing an extracellular matrix protein (e.g. collagen I) or a mixture of extracellular matrix proteins (e.g. a basement membrane extract). This mixture of epidermis-derived stem cells and extracellular matrix is dispensed as a drop on the bottom of a tissue culture petri dish or other appropriate receptacle. The extracellular matrix solution/mixture will subsequently solidify to a 3D-ECM when the pH is increased (for example from pH 3.0 to 7.5 in the case of collagen I) and/or when the temperature is increased (for example from 4° C. to 25° C. in the case of an extracellular matrix extract such as Matrigel). After solidification the droplet is covered by a cell culture media for culturing epidermis-derived stem cells according to the present invention.

For passaging cells are typically isolated from the 3D-ECM, for example, using the following protocol: The 3D-ECM mixture is mechanically disrupted by repeated aspiration with a pipet tip in a solution containing 0.5% Trypsin, 0.5 mM EDTA in PBS or alternatively in Accutase (Gibco). This is followed by further 10 min incubation at 37° C. in the said solutions. Cells are subsequently harvested by centrifugation, diluted in a ratio of 1:3-1:5 and re-suspended in a new mixture of ECM, which is subsequently transformed to a 3D-ECM by methods as described elsewhere herein. Preferably, cells are passaged every 10-14 days.

Culturing of epidermis-derived stem cells in particular refers to maintenance of these cells without losing typical features of epidermis-derived stem cells such as multipotency and/or the capacity to self-renew. In the context of culturing HFSCs it is particularly envisaged that these cells will be able to generate new HFSCs through cell division as well as to give rise to all differentiated cell types of the hair follicle and the interfollicular epidermis. Similarly, in the context of interfollicular epidermal stem cells it is particularly envisaged that these cells will be able to generate new interfollicular epidermal stem cells through cell division as well as to give rise to all differentiated cell types of the hair follicle and the interfollicular epidermis. Similarly, in the context of epidermis-derived cancer stem cells it is particularly envisaged that these cells will be able to generate new cancer stem cells through cell division as well as to give rise to all differentiated cell types of the tumor.

In one aspect culturing relates to maintaining epidermis-derived stem cells, which means the survival of said cells ex vivo in cell culture. Maintenance relates to a survival of the cells initially subjected to in vitro culturing according to the method of the current invention of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 100%. The number of surviving epidermis-derived stem cells can be determined by flow cytometry. In particular, epidermis-derived stem cells cultured according to the present invention are extracted from the 3D-ECM as described above and analyzed by a respective FACS analysis. For example, for analyzing HFSCs, single cell suspensions are then labeled with fluorescently labeled binding compounds that bind HFSCs, such as CD34 and α6 integrin. For CSCs, single cell suspensions are labeled with fluorescently-labeled binding compounds that bind, for example, CD140a, CD31, CD45, EpCAM, CD34 and α6 integrin. Labeled cells are analyzed using a flow cytometer to determine the proportion of epidermis-derived stem cells in respect to other cell populations.

More preferably, culturing epidermis-derived stem cells not only includes maintenance/survival of the cells subjected to culturing, but also relates to an expansion/growth of said cells. Expansion/growing means that the number of cells is increased during the time of in vitro culturing by mitotic cell division. In principle, expansion of the cultivated cells during culturing can be unlimited. Preferably, expansion means to increase the number of epidermis-derived stem cells by at least 1.5-fold, 2-fold, 5-fold, 10-fold, 20-fold, 50-fold, 100-fold or 200-fold during the time of culturing.

An “extracellular matrix” (ECM) is a collection of extracellular molecules secreted by cells that provides structural and biochemical support to the surrounding cells. In context of the present invention, the term “extracellular matrix” (ECM) means a collection of one or more extracellular molecules (as e.g, secreted by cells) that provides structural and biochemical support to the surrounding cells (in particular the cultured cells. Components/Extracellular molecules of an extracellular matrix (ECM) that are known in the art are, for example, a variety of polysaccharides, water, elastin and glycoproteins, wherein the glycoproteins comprise collagens, entactin(s) (also referred to as nidogen(s)), fibronectins, perlecan and/or laminins. More particular, an ECM in the context of the present invention, for example, may comprise and/or consist of: Collagen type I, Collagen type II, Collagen type III, Collagen type IV; Laminin-111, Laminin-211, Laminin-121, Laminin-221, Laminin-332, Laminin-3832, Laminin-311, Laminin-321, Laminin-411, Laminin-421, Laminin-511, Laminin-521, Laminin-213, Laminin-423, Laminin-522, Laminin-523, an Entactin/Nidogen (such as Nidogen 1, Nidogen 2), Perlecan, Vitronectin, and/or another Heparan sulfate proteoglycan than Perlecan such as agrin or Collagen XVIII.

As mentioned above, in the context of the method for culturing epidermis-derived stem cells according to the present invention, cells have to be embedded in a three-dimensional ECM (3D-ECM). In 3D-ECM cultures or 3D cultures, single cell suspensions are embedded within the ECM mixture prior to its solidification, allowing the cells to be embedded within the ECM from all sides. 3D-ECM can, for example, be generated by utilizing the natural properties of ECM components, in particular ECM proteins to self-assemble and to polymerize. 3D-ECM can also be produced by using synthetic polymers to which ECM components, in particular ECM proteins, or synthetic reactive groups of ECM components, in particular ECM proteins, are subsequently incorporated to as for example described in Rimann & Hauser, 2012, Curr Opin Biotech, 23: 1-7; Prestwich, 2007, J Cell Biochem 101: 1370-1383.

By contrast 2-dimensional (2D) culture conditions, 2D-ECM or 2D-culture conditions are achieved by coating a tissue culture petri dish with ECM components as listed above, allowing them to form a thin layer on top of the surface of the tissue culture receptacle and subsequently plating the cells on top of the ECM surface. 2D-ECM does therefore not comprise embedded cells. Further, it typically rather forms a thin 2D-layer on which cells are subsequently plated.

In the context of the present invention, in principle, any three-dimensional extracellular matrix (3D-ECM) can be employed. Different types of 3D-ECM with different compositions, in particular including different types of glycoproteins and/or different combinations of glycoproteins, are known in the art. Non-limiting examples are, for example, described in Hynes, R. O. and Naba, A. 2012. Cold Spring Harb. Perspect. Biol. 4, a004903.

In principle, an ECM/ECM components can be produced by culturing ECM-producing cells, such as, for example, fibroblast cells or various cancer-derived cell lines, such as, but not limited to M1536B3, PYS, HT1080, PF-HR9. In this regard, ECM-producing cells are cultured in a receptacle such as tissue culture petri dishes using standard culture conditions known in the art. ECM component production occurs throughout the period of culture, most typically between 2 and 20 days. The ECM components are secreted into the culture medium as soluble proteins from where they can be deposited as a soluble or insoluble protein meshwork, termed the extracellular matrix (ECM), into the bottom of the petri dish/receptacle. ECM proteins can be harvested directly from the culture medium as soluble proteins or by solubilizing the ECM meshwork using detergents such as Triton-X 100 or urea. ECM proteins can be further purified using methods known in the art for protein purification such as precipitation, ultracentrifugation, or chromatography (Yurchenco et al, 2002, Methods Cell Biol, 69:111-44). Such ECM protein(s), ECM meshwork, or further purified ECM protein(s) can subsequently be employed in the method for culturing epidermis-derived SCs according to the present invention. For this purpose, the cells are embedded in the ECM to form a 3D-ECM as described above in the context of culturing/passaging of cells.

Alternatively, the 3D-ECM can be assembled from purified proteins that have been purified from biological materials such as tissues or produced using methods of recombinant protein production in E. coli, P. pastoris, or other organisms suitable for protein production as known to a skilled person. These recombinant ECM proteins can, for example, be purchased commercially (e.g. provided by Invitrogen, Biolamina, or Millipore), or are recombinantly expressed and purified according to protocols well known in the art. Embedding of cells in such an ECM to form a 3D-ECM is achieved as described above in the context of culturing/passaging of cells. In the following it exemplary described how a collagen type I-based 3D-ECM with embedded cells in accordance with the method of the present invention is formed. The formation of 3D-ECMs comprising other or more ECM proteins and the embedding of cells therein is performed similarly. Only the respective proteins are exchanged. A collagen type I-based 3D-ECM to culture epidermis-derived stem cells is prepared by diluting commercially available, purified rat tail collagen type I (Millipore) in the desired growth medium (preferably one of the culturing media described by the current invention), mixing this dilution with epidermis-derived stem cells and then dispensing the mixture as a droplet on the bottom of a tissue culture petri dish, where it will subsequently solidify. The solidified 3D-ECM-cell mixture is subsequently covered by the culture medium described in the present invention. This way of culturing is typically also referred to as 3D-culturing.

As another and in the context of this invention preferred alternative, a commercially available ECM mixture may be employed, in particular for generating a 3D-ECM. Examples for commercially available ECMs are, for example, Matrigel™ (BD Biosciences), Cultrex™ (Amsbio), EHS Matrix™ (Sigma) or Geltrex™ (LifeTechnologies). All these commercially available ECMs are soluble forms of basement membrane purified from Engelbreth-Holm-Swarm (EHS) tumor and comprise: between 60% and 85% of laminins, between 5% and 30% collagen IV, between 1% and 10% nidogen, and 1 between 1% and 10% heparan sulfate proteoglycan. Thus, in the context of the method for culturing epidermis-derived stem cells according to the present invention preferably, a 3D-ECM is employed that comprises or consists of basement membrane proteins, preferably for example purified from Engelbreth-Holm-Swarm (EHS) tumor and composing between 60% and 85% of laminins, between 5% and 30% collagen IV, between 1% and 10% nidogen, and between 1% and 10% heparan sulfate proteoglycan. Such 3D-ECM is preferably generated by solidifying a initially soluble form of basement membrane proteins, preferably for example purified from Engelbreth-Holm-Swarm (EHS) tumor and composing between 60% and 85% of laminins, between 5% and 30% collagen IV, between 1% and 10% nidogen, and between 1% and 10% heparan sulfate proteoglycan to a 3D structure under defined conditions as described elsewhere herein.

Preferably, in the context of the present invention, a synthetic hydrogel ECM/3D-ECM or a naturally occurring ECM/3D-ECM can be employed. A synthetic 3D-ECM is generated with a commercially available ECM mixture as described above, or is assembled by one or more purified or recombinantly produced ECM components as described above. As mentioned above, the most preferred synthetic 3D-ECMs are generated with commercially available ECM components, e.g. soluble forms of basement membrane purified from Engelbreth-Holm-Swarm (EHS) tumor. Naturally occurring ECMs are preferably ECMs that are produced by ECM-producing cells as described above.

Preferably, in the context of the present invention, a 3D-ECM is employed that comprises one or more of the components selected from the group consisting of Collagen type I, Collagen type II, Collagen type III, Collagen type IV; Laminin-111, Laminin-211, Laminin-121, Laminin-221, Laminin-332, Laminin-3B32, Laminin-311, Laminin-321, Laminin-411, Laminin-421, Laminin-511, Laminin-521, Laminin-213, Laminin-423, Laminin-522, Laminin-523, a Entactin/nidogen (Nidogen 1 or Nidogen 2), Perlecan, Vitronectin, and/or another Heparan sulfate proteoglycan.

In particular, in the context of the present invention, a 3D-ECM comprising at least one ECM glycoprotein may be used. ECM glycoproteins are, for example, collagens, entactin(s) (nidogen(s)), fibronectins, perlecan, and/or laminins. The preferred ECM glycoprotein is at least one collagen or laminin. Most preferred the at least one ECM glycoprotein is a collagen.

Preferably, a method for culturing epidermis-derived stem cells according to the present invention employs a 3D-ECM that comprises at least two distinct glycoproteins. Particularly preferred is the use of a 3D-ECM comprising a laminin and a collagen. Even more preferred is the usage of a 3D-ECM comprising a laminin, a collagen and an entactin. Even more preferred is the employment of a 3D-ECM comprising a laminin, a collagen and an entactin, wherein said laminin is laminin-511 or laminin-332. Similarly, preferred is the employment of a 3D-ECM comprising a laminin, a collagen and an entactin, wherein said collagen is collagen IV. Most preferred is to employ a 3D-ECM that comprises: laminin-511, collagen IV and an entactin; or laminin-332, collagen IV and an entactin (also referred to as nidogen). Preferably, an entactin/nidogen is nidogen 1 or nidogen 2.

In the context of the present invention the term “basal cell culture medium” relates to a liquid medium that is known in the art for being suited for culturing human and/or animal cells. For example such basal cell culture medium can be a commercially available cell culture medium such as MEM (Spinners modification, Sigma) or DMEM. A preferred cell culture medium is a defined synthetic medium that is buffered at a pH of 7.4 (preferably between 7.2 and 7.6 or at least 7.2 and not higher than 7.6) with a carbonate-based buffer, while the cells are cultured in an atmosphere comprising between 5% and 10% CO₂, or at least 5% and not more than 10% CO₂, preferably 5% CO₂. A preferred cell culture medium can for example be selected from MEM that does not contain calcium, such as the Spinner's modification. Preferably, the basal cell culture medium comprises L-glutamine, insulin, hydrocortisone, penicillin, streptomycin, calcium-depleted fetal calf serum and/or calcium-depleted fetal bovine serum. Fetal bovine serum or fetal calf serum are preferably depleted of calcium, for example by chelating agents or dialysis. Supplements such as, for example, Ethanolamine and/or Phosphoethanolamine as supplements for phospholipid synthesis can further be added to the medium.

The basal cell culture medium may further comprise epidermal growth factor (EGF). EGF is a mitogenic growth factor that stimulates cell growth, proliferation, and differentiation by binding to its receptor EGFR. EGFs from different mammalian species are well known in the art and are for example described in (Edwin et al, 2006, Methods Mol Biol, 327:1-24). In general, EGF(s) of any mammalian organisms may be employed. Preferably, however, EGF(s) from the same organism from which the cultured epidermis-derived stem cells originate are employed. Similarly, EGF(s) of other organisms, which are known by the person skilled in the art to be biologically active in the organism from which the cultured epidermis-derived stem cells originate, are preferably employed. Accordingly, for example, in the context of culturing human epidermis-derived stem cells with the method of the current invention, human and/or murine EGF(s) originating from humans and/or mouse and/or most preferably resulting from recombinant protein production are employed.

Moreover, in principle, also (mutant) variants of EGF(s) known in the art such as LONG® EGF may be employed. Particularly preferred is LONG® EGF, a recombinant analogue of human that comprises the human EGF amino acid sequence plus a 53 amino acid N-terminal extension peptide. It is particularly preferred as it is animal-free, recombinantly expressed in E-coli. It can, for example, replace human, murine or recombinant EGF and it is already used in approved and marketed cell-based therapies. LONG® EGF is commercially available, for example from Repligen Inc.

EGF is preferably comprised in the basal cell culture medium employed in the method for culturing epidermis-derived stem cells according to the present invention at a concentration of between 2 ng/ml and 500 ng/ml or of at least 2 ng/ml and/or not higher than 500 ng/ml. A preferred concentration is at least 5, 10, 20, 25, 30, 40, 45, or 50 ng/ml and/or not higher than 500, 450, 400, 350, 300, 250, 200, 150, or 100 ng/ml. A more preferred concentration is between 5 ng/ml and 100 ng/ml. A more preferred concentration is also at least 5 ng/ml and/or not higher than 100 ng/ml. An even more preferred concentration is between 5 ng/ml and 100 ng/ml. An even more preferred concentration is about 20 ng/ml, or exactly 20 ng/ml.

Similarly, the basal cell culture medium may comprise Vascular Endothelial Growth Factor(s) (VEGF(s)). The VEGF family comprises of five members in mammals (VEGF-A, placenta growth factor (PGF), VEGF-B, VEGF-C and VEGF-D) and VEGFs are produced by cells that stimulate vasculogenesis and angiogenesis. In the context of the present invention the term VEGF in particular refers to the VEGF-A. Accordingly, if the term VEGF is used elsewhere herein, VEGF-A is preferably meant. VEGF's (VEGF-A) normal function is to create new blood vessels in different occasions such as for example during embryonic development, after injury, in the process of muscle formation following exercise and the bypass of blocked vessels. VEGF is also used by tumor cells to induce neovascularization within tumors. VEGF stimulates cellular responses by binding to VEGF receptors (the VEGFRs), which are tyrosine kinase receptors on the cell surface. The VEGF receptors have an extracellular portion consisting of 7 immunoglobulin-like domains, a single transmembrane spanning region, and an intracellular portion containing a split tyrosine-kinase domain. VEGF-A binds to VEGFR-1 (Flt-1)) and VEGFR-2 (KDR/Flk-1), however, VEGFR-2 appears to mediate almost all of the known cellular responses to VEGF.

VEGF (VEGF-A) exists in multiple isoforms that result from alternative splicing of mRNA from the VEGF (VEGF-A) gene. VEGFs and their isoforms from different mammalian species are well known in the art and are for example described in Chung & Ferrara, 2011, Annu Rev Cell Dev Biol 27: 563-584. In the context of the present invention, in principle, any VEGF isoform(s) originating from any mammalian organisms may be employed. Preferably, however, VEGF(s) from the same organism from which the cultured epidermis-derived stem cells originate are employed. Similarly, VEGF(s) of other organisms, which are known to be biologically active in the organism from which the cultured epidermis-derived stem cells originate, are preferably employed. Accordingly, for example, in the context of culturing human epidermis-derived stem cells with the method of the current invention, human and/or murine VEGF(s) originating from humans and/or mouse or most preferably from recombinant protein production are employed.

In particular, for culturing epidermis-derived stem cells VEGF(s), which is/are selected from the group of VEGF isoforms consisting of VEGF-121, VEGF-120, VEGF-145, VEGF-165, VEGF-164, VEGF-183, VEGF-188, VEGF-189 and VEGF-206 (Holmes and Zachary. 2005. Genome Biol 6:209), is (are) preferably employed. Preferably, VEGF-188, VEGF-164 and/or VEGF-120 is/are murine. Preferably, VEGF-121, VEGF-145, VEGF-165, VEGF-183, VEGF-189 and/or VEGF-206 is/are human. Preferably, VEGF(s) selected from the group of VEGF isoforms consisting of VEGF-164, VEGF-165, VEGF-121, VEGF-120 and VEGF-188 is/are employed. Most preferably, the isoform(s) VEGF-164 and VEGF-121 is (are) employed as VEGF(s).

Moreover, in principle, also (mutant) variants of VEGF(s) known in the art such as chimeric molecules consisting of mosaic of VEGF-A NEGF-C or VEGF-A/ENZ7 may be employed (Jeltsch et al, 2006, J Biol Chem, 281:12187-12195; Zheng et al, 2007, Aterioscler Thromb Vasc Biol, 27: 503-511).

A VEGF is preferably comprised in the basal cell culture medium employed in the method for culturing epidermis-derived stem cells according to the present invention at a total concentration of between 2 ng/ml and 500 ng/ml or of at least 2 and/or not higher than 500 ng/ml. A preferred concentration is at least 2, 5, 10, 20, 25, 30, 40, 45, or 50 ng/ml and/or not higher than 500, 450, 400, 350, 300, 250, 200, 150, or 100 ng/ml. A more preferred concentration is between 5 ng/ml and 100 ng/ml. A more preferred concentration is also at least 5 ng/ml and/or not higher than 100 ng/ml. An even more preferred concentration is between 5 ng/ml and 100 ng/ml. An even more preferred concentration is about 20 ng/ml, or even more preferred exactly 20 ng/ml.

If more than one VEGF (e.g. different VEGF isoforms and/or VEGFs originating from different organisms) is employed, the concentration of a VEGF is as defined above and refers to the total concentration of VEGFs used, i.e. the total sum of VEGF concentrations (e.g. if VEGF-164 and VEGF-165 are employed, the sum of the concentrations of VEGF-164 and VEGF-165). In such a case the above-mentioned values apply only to the sum of VEGF concentrations and do not mean that all of the VEGFs need to be present in the above mentioned concentration ranges. For example, at least 5 ng/ml of total VEGFs would be present if the basal cell culture medium comprises 3 ng/ml VEGF-164 and 2 ng/ml VEGF-165.

Furthermore, in the context of the present invention, the basal cell culture medium may comprise Fibroblast Growth Factor(s) (FGF(s)). FGFs are a family of mitogenic growth factors, with members involved in angiogenesis, wound healing, embryonic development and various endocrine signaling pathways. The FGFs are heparin-binding proteins and interactions with cell-surface-associated heparan sulfate proteoglycans have been shown to be essential for FGF signal transduction. FGFs are key players in the processes of proliferation and differentiation of wide variety of cells and tissues. In mammals, for examples the following members of the FGF family are known: FGF-1, FGF-2, FGF-3, FGF-4, FGF-5, FGF-6, FGF-7, FGF-10, FGF-22, FGF-8, FGF-17, FGF-18, FGF-24, FGF-9, FGF-16, FGF-20, FGF-11, FGF-12, FGF-13, FGF-14, FGF-19, FGF-21, and FGF-23. FGFs from different mammalian species are well known in the art and are, for example, described in Ornitz & Itoh, 2001, Genome Biol 2: 3005.1-3005.12. FGF(s) function is mediated by its (their) binding to so called fibroblast growth factor receptor(s) (FGFRs). The mammalian fibroblast growth factor receptor family has 4 members, FGFR1, FGFR2, FGFR3, and FGFR4.

Generally, in the context of the embodiments of the method for culturing epidermis-derived stem cells according to the present invention any FGF(s) of any mammalian organism may be employed. In particular, FGF(s) is/are preferably selected from the group consisting of FGF-1, FGF-2, FGF-3, FGF-4, FGF-5, FGF-6, FGF-7, FGF-10, FGF-22, FGF-8, FGF-17, FGF-18, FGF-24, FGF-9, FGF-16, FGF-20, FGF-11, FGF-12, FGF-13, FGF-14, FGF-19, FGF-21, and FGF-23. Preferably, FGF-2, FGF-7, FGF-10 and/or FGF-18 are employed. Most preferred, FGF-2 and/or FGF-18 are employed. In general, FGF(s) from any mammalian organisms may be employed. Preferably, however, FGF(s) from the same organism from which the cultured epidermis-derived stem cells originate is/are employed. Similarly, FGF(s) of other organisms, which is/are known to be biologically active in the organism from which the cultured epidermis-derived stem cells originate, is/are preferably employed. In the context of culturing epidermis-derived stem cells (preferably human or murine) in the present invention, for example, human and/or murine FGF(s) originating from humans or mouse or most preferably from recombinant protein production are employed.

Moreover, in principle, also (mutant) variants of FGF(s) known in the art can be employed. These include, but are not restricted to a heat-stable chimeric variant of FGF, termed FGFC. FGFC is a chimeric protein composed of human FGF1 and FGF2 domains that exhibits higher thermal stability and protease resistance than do both FGF-1 and FGF-2 (Onuma et al., 2015, Plos One 10:e0118931).

A FGF is preferably comprised in the basal cell culture medium employed in the method for culturing epidermis-derived stem cells according to the present invention at a concentration of between 2 ng/ml and 500 ng/ml or of at least 2 ng/ml and/or not higher than 500 ng/ml. A preferred concentration is at least 2, 5, 10, 20, 25, 30, 40, 45, or 50 ng/ml and/or not higher than 500, 450, 400, 350, 300, 250, 200, 150, or 100 ng/ml. A more preferred concentration is between 5 ng/ml and 100 ng/ml. A more preferred concentration is also at least 5 ng/ml and/or not higher than 100 ng/ml. An even more preferred concentration is between 5 ng/ml and 100 ng/ml. An even more preferred concentration is about 20 ng/ml, or even more preferred exactly 20 ng/ml.

If more than one FGF (e.g. different members of the FGF family and/or FGFs originating from different organisms) is employed, the concentration of a FGF is as defined above and refers to the total concentration of FGFs used, i.e. the total sum of FGF concentrations (e.g. if FGF-2 and FGF-7 are used, the sum of the concentrations of FGF-2 and FGF-7). In such a case the above mentioned values apply only to the sum of FGF concentrations and do not mean that all of the FGFs need to be present in the above mentioned concentration ranges. For example, at least 5 ng/ml of total FGFs would be present if the basal cell culture medium comprises 3 ng/ml FGF-2 and 2 ng/ml FGF-7.

According to the present invention, a basal cell culture medium comprising an inhibitor of the Rho-kinase (ROCK), referred to as ROCK inhibitor/antagonist is employed.

Rho-kinase (ROCK) is a kinase of the serine-threonine kinase family. It is involved mainly in regulating the shape and movement of cells by acting on the cytoskeleton. ROCK exists in two homologous, ROCK1 and ROCK2. ROCKs (ROCK1 and ROCK2) occur in mammals (human, rat, mouse, cow), zebrafish, Xenopus, invertebrates (C. elegans, Mosquito, Drosophila) and chicken and are, for example described in Julian & Olson, 2014, Small GTPases e29846. doi: 10.4161/sgtp.29846. The addition of a Rock inhibitor/antagonist was found to prevent anoikis that is a form of programmed cell death that is induced by anchorage-dependent cells detaching from the surrounding extracellular matrix (ECM).

A “ROCK inhibitor”, “ROCK antagonist” or “ROCK inhibitor/antagonist” is a chemical substance, which inhibits the kinase activity of ROCK1 (also known as p160ROCK) and/or ROCK2. Most preferred a “ROCK inhibitor”, “ROCK antagonist” or “ROCK inhibitor/antagonist” inhibits ROCK1. Preferably, the kinase activity of ROCK 1 and/or ROCK2 is inhibited by at least 10%, at least 20%, at least 30%, at least 40%, at least 50%, at least 60%, at least 70%, at least 80%, at least 90%, or at least 95% or at least 99%. Preferably the inhibition/reduction in activity of ROCK1 and/or ROCK2 is high, but only as high as it does not have significant negative impacts on cell viability. The inhibition of ROCK1 and/or ROCK2 can be measured by methods known in the art, like e.g. in vitro kinase activity assays or in vivo measurement of the phosphorylation status of one or more ROCK substrates. An example for a ROCK substrate is the myosin light chain. Thus, for example, myosin light chain phosphor-specific antibodies may be used in assays like western blotting to detect the reduction of phosphorylation, i.e. the inhibition/reduction in activity of ROCK1 and/or ROCK2 (Julian & Olson, 2014, Small GTPases e29846. doi: 10.4161/sgtp.29846).

In the context of the present invention, in principle any ROCK inhibitor can be employed. Preferably, the ROCK inhibitor(s) is (are) selected from the group consisting of (R)-(+)-trans-4-(1-aminoethyl)-N-(4-Pyridyl)cyclohexanecarboxamide dihydrochloride monohydrate (referred to as Y-27632), 5-(1,4-diazepan-1-ylsulfonyl) isoquinoline (referred to as fasudil or HA 1077), and (S)-(+)-2-methyl-1-[(4-methyl-5-isoquinolinyl)sulfonyl]-hexahydro-1H-1,4-diazepine dihydrochloride (referred to as H-1152). Most preferably, Y-27632 is employed in the method of culturing epidermal-derived stem cells as mentioned above.

In principle, every concentration of a ROCK inhibitor that results in inhibition of ROCK, as defined and measured by the methods described above, can be used. Preferably, a ROCK inhibitor is comprised in the basal cell culture medium at a total concentration of between 2 μM and 20 μM, preferably of between 3 μM and 15 μM or most preferably of between 5 to 10 μM or most preferably 5 μM.

If more than one ROCK inhibitor (e.g. Y-27632 and HA 1077) is employed, the concentration of a ROCK inhibitor is as defined above and refers to the total concentration of ROCK inhibitors used, i.e. the total sum of ROCK inhibitor concentrations (e.g. if Y-27632 and HA 1077 are used, the sum of the concentrations of Y-27632 and HA 1077). In such a case the above mentioned values apply only to the sum of ROCK inhibitor concentrations and do not mean that all of the ROCK inhibitors need to be present in the above mentioned concentration ranges. For example, at least 5 μM of total ROCK inhibitors would be present if the basal cell culture medium comprises 3 μM Y-27632 and 2 μM HA 1077.

Rho-kinase (ROCK) inhibitor(s), EGF, VEGF(s), FGF(s), and/or any other supplements of the basal cell culture medium may be added to the culture medium in regular time frames, e.g. every, every third, every forth or preferably every second day. Preferably, it/they are added together with fresh cell culture medium, which is, as described above, regularly (e.g. every second day) provided.

In the context of the present invention it is preferred that the Rho-kinase (ROCK) inhibitor(s), EGF, VEGF(s), FGF(s), and/or any other supplements of the basal cell culture medium are present in the culture medium during the complete culturing.

In the context of the present invention it is, however, in principle also possible to culture and/or expand epidermis-derived stem cells in the absence of ROCK inhibitors after the cells have been cultured and/or expanded (also referred to as pre-culturing or initial culturing) for at least 2 days, preferably at least 3 days, more preferably at least 4 days, even more preferably at least 5 days, even more preferably at least 6 days, and most preferably at least 7 days according to the methods of the present invention using a basal cell culture medium comprising a Rho-kinase (ROCK) inhibitor(s) and EGF, VEGF(s), FGF(s), and/or any other supplements. In the pre-culturing or initial culturing preferably Y-27632 is employed as ROCK inhibitor. Most preferably, Y-27632 is employed in the concentrations as defined elsewhere herein for the ROCK inhibitor(s) and/or Y27632. As shown in the appended Examples and Figures, the presence of a ROCK inhibitor is only absolutely required during the first two days of culturing and/or expanding epidermis-derived stem cells comprised in freshly isolated keratinocytes. After the initial two days of culturing the presence of ROCK inhibitor(s) is/are not absolutely required to maintain or increase the proportion of epidermis-derived stem cells in the cultured cells. However, the continuous presence of ROCK inhibitor is still preferred, because in the absence of ROCK inhibitor(s) after the initial culturing for at least 2 days (in presence of ROCK inhibitor(s)) the absolute numbers of epidermis-derived stem cells decreases over time. An advantage of the absence of ROCK inhibitor(s) after two days of culturing may nevertheless be advantageous as regarding reducing costs and efforts for providing ROCK inhibitor(s) or in case the cultures are being used to study the actomyosin cytoskeleton, whereby inhibiting ROCK might not be desired. In view of the above, the present invention also relates to a method for culturing epidermis-derived stem cells comprising the step of culturing epidermis-derived stem cells in the presence of a three-dimensional extracellular matrix (3D-ECM) and a basal cell culture medium comprising:
Epidermal Growth Factor (EGF); and/or
a Vascular Endothelial Growth Factor (VEGF); and/or
a Fibroblast Growth Factor (FGF);
   wherein said epidermis-derived stem cells are pre-cultured prior to said step of culturing for at least 2 days in the presence of a three-dimensional extracellular matrix (3D-ECM) and a basal cell culture medium comprising:
Epidermal Growth Factor (EGF); and/or
a Vascular Endothelial Growth Factor (VEGF); and/or
a Fibroblast Growth Factor (FGF);
and further a Rho-kinase (ROCK) inhibitor (preferably Y-27632).

In other words, the present invention also provides for a method for culturing epidermis-derived stem cells comprising:

a first step of culturing epidermis-derived stem cells for at least 2 days in the presence of a three-dimensional extracellular matrix (3D-ECM) and a basal cell culture medium comprising:
Epidermal Growth Factor (EGF); and/or
a Vascular Endothelial Growth Factor (VEGF); and/or
a Fibroblast Growth Factor (FGF);
and further a Rho-kinase (ROCK) inhibitor (preferably Y-27632); and
   a subsequent second step of culturing epidermis-derived stem cells after said least 2 days in the presence of a three-dimensional extracellular matrix (3D-ECM) and a basal cell culture medium comprising:
Epidermal Growth Factor (EGF); and/or
a Vascular Endothelial Growth Factor (VEGF); and/or
a Fibroblast Growth Factor (FGF).

Preferably, in this context the second step may be no longer than 10 days, preferably no longer than 9 days, more preferably no longer than 8 days, more preferably no longer than 7 days, more preferably no longer than 6 days, more preferably no longer than 5 days, more preferably no longer than 4 days, more preferably no longer than 3 days and most preferably no longer than 2 days.

The concept that ROCK inhibitor(s) are only absolutely required for the first 2 days of culturing and can subsequently be omitted from the employed basal cell culture medium can also be applied mutatis mutandis in the methods for de novo generation of epidermis-derived stem cells disclosed in the present invention. Accordingly, also corresponding methods for de novo generation of epidermis-derived stem cells employing either cells pre-cultured for at least 2 days as described above or having the above-mentioned first and second steps are disclosed herein.

The method for culturing epidermis-derived stem cells of the present invention employs, inter alia, a basal cell culture medium comprising EGF and/or a VEGF and/or a FGF and a ROCK inhibitor. EGF, VEGF(s) and FGF(s) are known to be mitogenic growth factors. Accordingly, in other words the basal cell culture medium employed in the method of the current invention preferably comprises: a) at least one mitogenic growth factor selected from the group consisting of EGF, a VEGF and a FGF; and b) a ROCK inhibitor.

Accordingly, the method of culturing epidermis-derived stem cells may employ a basal cell culture medium, which comprises a VEGF and a ROCK inhibitor, a FGF and a ROCK inhibitor or preferably EGF and a ROCK inhibitor. In other words, the cell culture medium preferably comprises: a) one mitogenic growth factor selected from the group consisting of EGF, a VEGF, and a FGF; and b) a ROCK inhibitor.

Preferably, the method of culturing epidermis-derived stem cells employs a basal cell culture medium, which comprises EGF, a VEGF and a ROCK inhibitor; EGF, a FGF and a ROCK inhibitor; or a VEGF, a FGF and a ROCK inhibitor. In other words, the basal cell culture medium employed in the method of the current invention preferably comprises: a) at least two mitogenic growth factors selected from the group consisting of EGF, a VEGF, and a FGF; and b) a ROCK inhibitor.

Furthermore, the method for culturing epidermis-derived stem cells preferably employs a basal cell culture medium comprising: EGF and a VEGF and/or a FGF and a ROCK inhibitor. In other words, the basal cell culture medium employed in the method of the current invention preferably comprises: EGF; at least one mitogenic growth factor selected from the group consisting of the VEGFs VEGF-121, VEGF-120, VEGF-145, VEGF-165, VEGF-164, VEGF-183, VEGF-188, VEGF-189 and VEGF-206 and the FGFs FGF-1, FGF-2, FGF-3, FGF-4, FGF-5, FGF-6, FGF-7, FGF-10, FGF-22, FGF-8, FGF-17, FGF-18, FGF-24, FGF-9, FGF-16, FGF-20, FGF-11, FGF-12, FGF-13, FGF-14, FGF-19, FGF-21, and FGF-23; and a ROCK inhibitor. Preferably the group of VEGFs consists of VEGF-121 and VEGF-164. Similarly, the group of FGFs preferably consists of FGF-2, FGF-7, FGF-10 and FGF-18. Even more preferably, the group of FGFs consists of FGF-2 and FGF-18.

In particular, the basal cell culture medium preferably comprises: EGF, a VEGF selected from the group consisting of VEGF-164 and VEGF-121, and further a ROCK inhibitor (preferably Y-27632); or EGF, a FGF selected from the group consisting of FGF-2, FGF-7, FGF-10 and FGF-18, and further a ROCK inhibitor (preferably Y-27632); or a VEGF selected from the group consisting of VEGF-164 and VEGF-121, a FGF selected from the group consisting of FGF-2, FGF-7, FGF-10 and FGF-18, and further a ROCK inhibitor (preferably Y-27632). Also in this context the group of FGFs consists even more preferably of FGF-2 and FGF-18.

Thus, the method for culturing epidermis-derived stem cells preferably employs a basal cell culture medium comprising: EGF, FGF-2, and a ROCK inhibitor (preferably Y-27632); or EGF, FGF-7, and a ROCK inhibitor (preferably Y-27632); or EGF, FGF-10, and a ROCK inhibitor (preferably Y-27632); or EGF, FGF-18, and a ROCK inhibitor (preferably Y-27632); or EGF, VEGF-164, and a ROCK inhibitor (preferably Y-27632); or EGF, VEGF-121, and a ROCK inhibitor (preferably Y-27632); or FGF-2, VEGF-121 and a ROCK inhibitor (preferably Y-27632).

Even more preferably the method for culturing epidermis-derived stem cells, as mentioned above, employs a basal cell culture medium, which comprises: EGF; a VEGF; a FGF and a ROCK inhibitor. In other words, in a preferred embodiment the basal cell culture medium employed in the method of the current invention comprises: EGF; a VEGF selected from the group consisting of VEGF-121, VEGF-120, VEGF-145, VEGF-165, VEGF-164, VEGF-183, VEGF-188 and VEGF-206; a FGF selected from the group consisting of FGF-1, FGF-2, FGF-3, FGF-4, FGF-5, FGF-6, FGF-7, FGF-10, FGF-22, FGF-8, FGF-17, FGF-18, FGF-24, FGF-9, FGF-16, FGF-20, FGF-11, FGF-12, FGF-13, FGF-14, FGF-19, FGF-21, and FGF-23; and a ROCK inhibitor.

In particular, the method for culturing epidermis-derived stem cells according to the present invention preferably employs a basal cell culture medium comprising: EGF; a VEGF selected from the group consisting of VEGF-164 and VEGF-121; and a FGF selected from the group consisting of FGF-2, FGF-7, FGF-10 and FGF-18; and a ROCK inhibitor (preferably Y-27632). Preferably, a FGF selected from the group consisting of FGF-2 and FGF-18 is employed.

Most preferably, the method for culturing epidermis-derived stem cells, as mentioned above, employs a basal cell culture medium, which comprises: EGF, VEGF-164, FGF-2 and a ROCK inhibitor (preferably Y-27632); or EGF, VEGF-164, FGF-18 and a ROCK inhibitor (preferably Y-27632); or EGF, VEGF-121, FGF-2 and a ROCK inhibitor (preferably Y-27632); or EGF, VEGF-121, FGF-18 and a ROCK inhibitor (preferably Y-27632); or EGF, VEGF-164, FGF-7 and a ROCK inhibitor (preferably Y-27632); or EGF, VEGF-164, FGF-10 and a ROCK inhibitor (preferably Y-27632); or EGF, VEGF-121, FGF-7 and a ROCK inhibitor (preferably Y-27632); or EGF, VEGF-121, FGF-10 and a ROCK inhibitor (preferably Y-27632). It is particularly preferred that the method for culturing epidermis-derived stem cells, as mentioned above, employs a basal cell culture medium, which comprises: EGF, VEGF-164, FGF-2 and the ROCK inhibitor Y-27632; or EGF, VEGF-164, FGF-18 and the ROCK inhibitor Y-27632; or EGF, VEGF-121, FGF-2 and the ROCK inhibitor Y-27632; or EGF, VEGF-121, FGF-18 and the ROCK inhibitor Y-27632; or EGF, VEGF-164, FGF-7 and the ROCK inhibitor Y-27632; or EGF, VEGF-164, FGF-10 and the ROCK inhibitor Y-27632; or EGF, VEGF-121, FGF-7 and the ROCK inhibitor Y-27632; or EGF, VEGF-121, FGF-10 and the ROCK inhibitor Y-27632. Similarly, it is particularly preferred that the method for culturing epidermis-derived stem cells, as mentioned above, employs a basal cell culture medium, which comprises: EGF, VEGF-164, FGF-2 and the ROCK inhibitor Y-27632. It is also particularly preferred that the method for culturing epidermis-derived stem cells, as mentioned above, employs a basal cell culture medium, which comprises: EGF, VEGF-121, FGF-2 and the ROCK inhibitor Y-27632.

In another preferred embodiment of the method for culturing epidermis-derived stem cells according to the present invention, a basal cell culture medium as described herein is employed which further comprises a Sonic Hedgehog (SHH) inhibitor/antagonist.

The term “Sonic Hedgehog (SHH)” in the context of SHH inhibitor/antagonist refers to the Sonic Hedgehog (SHH) signaling pathway. Dysregulation of this pathway is usually lethal in early embryonic stages. Mutations in the SHH pathway have been identified in a large variety of malignant tumors. The protein Hedgehog is the extracellular component of the pathway and activates intracellular signals after binding to its specific receptor “Patched” (Ptch), a protein located on the cellular membrane. After binding of Hedgehog to Patched, a protein called “Smoothened” (SMO) becomes activated and thus induces transcription of target genes of the Hedgehog pathway. In the absence of Hedgehog the activity of Smoothened is suppressed by Patched and thus the target genes of the Hedgehog pathway are not expressed.

Thus, the term “SHH inhibitor/antagonist”, “SHH inhibitor” or “SHH antagonist” in the context of the present invention is defined as a chemical compound targeting components of the SHH signaling pathway, thus inhibiting its activity/signaling activity. Preferably, the activity/signaling activity is inhibited by at least 10%, at least 20%, at least 30%, at least 40%, at least 50%, at least 60%, at least 70%, at least 80%, at least 90%, at least 95% or at least 99%. In principle, higher values, i.e. stronger inhibition/reduction in activity/signaling activity of the SHH signaling pathway, are preferred. The inhibition of the SHH signaling pathway can be measured by methods known in the art, like e.g. measurement of the expression levels of the target genes and the corresponding encoded proteins by methods that are well known in the art. In particular, measuring mRNA levels of the SHH target genes Patched1 and Gli1-3 are preferred (Varjosalo & Taipale, 2008, Genes Dev 22: 2454-2472).

In principle, any SHH inhibitor/antagonist can be employed in the method for culturing epidermis-derived stem cells according to the present invention. In particular, in the context of the present invention a SHH inhibitor/antagonist is employed, which is selected from the group consisting of (2′R,3S,3′R,3′aS,6'S,6aS,6bS,7′aR,11aS,11bR)-1,2,3,3′a,4,4′,5′,6,6′,6a,6b,7,7′,7′a,8,11,11a,11b-Octadecahydro-3′,6′,10,11b-tetramethylspiro[9H-benzo[a]fluorene-9,2′(3′H)-furo[3,2-b]pyridin]-3-ol (referred to as Cyclopamine); 2-chloro-N-(4-chloro-3-pyridin-2-ylphenyl)-4-methylsulfonylbenzamide (referred to as HhAntag691); N-((2S,3R,3aS,3′R,4a′R,6S,6a′R,6b'S,7aR,12a'S,12b'S)-3,6,11′,12b′-tetramethyl-2′,3a,3′,4,4′,4a′,5,5′,6,6′,6a′,6b′,7,7a,7′,8′,10′,12′,12a′,12b′-icosahydro-1′H,3H-spiro[furo[3,2-b]pyridine-2,9′-naphtho[2,1-a]azulen]-3′-yl)methanesulfonamide; (referred to as IPI-926); N-[6-[(2S,6R)-2,6-dimethylmorpholin-4-yl]pyridin-3-yl]-2-methyl-3-[4-(trifluoromethoxy)phenyl]benzamide (referred to as LDE225); and N-[(4-chlorophenyl)methyl]-2-[(2R,6S,8E)-5,12-dioxo-2-phenyl-1-oxa-4-azacyclododec-8-en-6-yl]acetamide (referred to as Robotnikinin). At least some of these SHH inhibitors/antagonists are commercially available. Preferably, in the method for culturing epidermis-derived stem cells according to the present invention, the SHH inhibitor cyclopamine is employed.

In general, every concentration of a SHH inhibitor that results in inhibition of the SHH signaling pathway, as defined and measured by the methods described above, can be used. Preferably, a SHH inhibitor is comprised in the basal cell culture medium at a total concentration of between 1 μM and 20 μM, preferably of between 5 μM and 15 μM, more preferably of between 5 μM and 10 μM and most preferably of about 10 μM or 10 μM. These concentration range(s) or concentration(s) is/are particularly preferred if cyclopamine is used as a SHH inhibitor.

If more than one SHH inhibitor (e.g. Cyclopamine and HhAntag691) is employed, the concentration of a SHH inhibitor is as defined above and refers to the total concentration of SHH inhibitors used, i.e. the total sum of SHH inhibitor concentrations (e.g. if Cyclopamine and HhAntag691 are used, the sum of the concentrations of Cyclopamine and HhAntag691). In such a case the above mentioned values apply only to the sum of SHH inhibitor concentrations and do not mean that all of the SHH inhibitors need to be present in the above mentioned concentration ranges. For example, at least 5 μM of total SHH inhibitors would be present if the basal cell culture medium comprises 3 μM Y-27632 and 2 μM HA 1077.

Accordingly, the present invention also relates to a method for culturing epidermis-derived stem cells comprising the step of culturing epidermis-derived stem cells in the presence of a three-dimensional extracellular matrix (3D-ECM) and a basal cell culture medium comprising:
Epidermal Growth Factor (EGF); and/or
a Vascular Endothelial Growth Factor (VEGF); and/or
a Fibroblast Growth Factor (FGF);
and further a ROCK (Rho-kinase) inhibitor,
wherein said basal cell culture medium further comprises a Sonic Hedgehog (SHH) inhibitor.

Even more preferably, the basal cell culture medium in this context comprises:
Epidermal Growth Factor (EGF); and
a Vascular Endothelial Growth Factor (VEGF); and/or
a Fibroblast Growth Factor (FGF);
and further a ROCK (Rho-kinase) inhibitor,
wherein said basal cell culture medium further comprises a Sonic Hedgehog (SHH) inhibitor.

Most preferably in this context, the basal cell culture medium in this context comprises:
Epidermal Growth Factor (EGF); and
a Vascular Endothelial Growth Factor (VEGF); and
a Fibroblast Growth Factor (FGF);
and further a ROCK (Rho-kinase) inhibitor,
wherein said basal cell culture medium further comprises a Sonic Hedgehog (SHH) inhibitor.

In other words, the present invention also relates to a method for culturing epidermis-derived stem cells comprising the step of culturing epidermis-derived stem cells in the presence of a three-dimensional extracellular matrix (3D-ECM) and a basal cell culture medium comprising: a) at least one mitogenic growth factor selected from the group consisting of EGF, a VEGF and a FGF; and b) a ROCK inhibitor; and c) a SHH inhibitor. More preferably, in this context a basal cell culture medium comprising: a) at least two mitogenic growth factors selected from the group consisting of EGF, a VEGF and a FGF; and b) a ROCK inhibitor; and c) a SHH inhibitor.

The present invention also provides a method for culturing epidermis-derived stem cells according to the present invention, wherein a basal cell culture medium as described anywhere elsewhere herein is employed which does not comprise a Bone Morphogenetic Protein (BMP) inhibitor/antagonist. In principle, it is preferred to employ a cell culture medium that does not comprise any BMP inhibitor/antagonist. In particular it is envisaged that no BMP inhibitor(s) selected from the group consisting of Noggin, chordin, gremlin, crossveinless, USAG-1, follistatin, Dorsomorphin, K02288, DMH.1, ML 347, LDN-193189 is comprised in the basal cell culture medium in the context of the method for culturing epidermis-derived stem cells according to the present invention.

Bone morphogenetic proteins (BMPs) are a group of growth factors also known as cytokines and as metabologens. Originally discovered by their ability to induce the formation of bone and cartilage, BMPs are now considered to constitute a group of pivotal morphogenetic signals, orchestrating tissue architecture throughout the body. Known members of the BMP protein family are for example: BMP1, BMP2, BMP2a, BMP3, BMP3b, BMP4, BMP5, BMP6, BMP7, BMP8a, BMP8b, BMP10, BMP15.

The term “BMP inhibitor/antagonist”, “BMP inhibitor” or “BMP antagonist” in the context of the present invention is defined as a chemical compound and/or a protein with inhibitory activity towards the activity/signaling activity of one or more BMP proteins. Such an inhibitory protein could, for example be Noggin, chordin, gremlin, crossveinless, USAG-1 and follistatin (Walsh et al., 2010 Trends Cell Biol 20:244-256). Preferably, a BMP inhibitor is a compound that inhibits the activity/signaling activity of one or more BMP by at least 20%, at least 30%, at least 40%, at least 50%, at least 60%, at least 70%, at least 80%, at least 90%, at least 95% or at least 99%. In principle, higher values, i.e. stronger inhibition/reduction in activity/signaling activity of the BMP activity/signaling activity, are a preferred definition. The inhibition of the BMP activity/signaling activity can be measured by methods known in the art, like e.g. measurement of the expression levels of the target genes and the corresponding encoded proteins by methods that are well known in the art, and/or by measuring the activity status of the pathway proteins. In particular, analyzing the expression of BMP target genes such as Id1, Id2, and Id3 or measuring the phosphorylation status of BMP pathway signaling components, Smad 1, 5, and 8 (Walsh et al., 2010, Trends Cell Biol 20:244-256) may be used. Alternatively, the transcriptional activity of BMP, as for example exemplified in Zilberberg et al. 2007, BCM Cell Biol 8:41.

The present invention also relates to a method for culturing epidermis-derived stem cells according to the present invention, wherein a basal cell culture medium as described anywhere elsewhere herein is employed which does not comprise a Bone Morphogenetic Protein (BMP) inhibitor/antagonist and/or a Wnt agonist. Similarly, the present invention further relates a method for culturing epidermis-derived stem cells according to the present invention, wherein a basal cell culture medium as described anywhere elsewhere herein is employed which does not comprise a Wnt agonist.

The Wnt signaling pathway is defined by a series of events that occur when a Wnt protein binds to a cell-surface receptor of a Frizzled receptor family member. This results in the activation of Dishevelled family proteins which inhibit a complex of proteins that includes axin, GSK-3, and the protein APC to degrade intracellular β-catenin. The resulting enriched nuclear β-catenin enhances transcription by TCF/LEF family transcription factors. A Wnt agonist is defined as an agent that activates TCF/LEF-mediated transcription in a cell. Wnt agonists are therefore selected from true Wnt agonists that bind and activate a Frizzled receptor family member including any and all of the Wnt family proteins, an inhibitor of intracellular β-catenin degradation, and activators of TCF/LEF. Said Wnt agonist stimulates a Wnt activity in a cell by at least 10%, more preferred at least 20%, more preferred at least 30%, more preferred at least 50%, more preferred at least 70%, more preferred at least 90%, more preferred at least 99%, relative to a level of said Wnt activity in the absence of said molecule. As is known to a skilled person, a Wnt activity can be determined by measuring the transcriptional activity of Wnt, for example by pTOPFLASH and pFOPFLASH Tcf luciferase reporter constructs (Korinek et al, 1997 Science 275 1784-1787).

For example, a Wnt agonist is or comprises a secreted glycoprotein such as: Wnt-I/Int-1, Wnt-2/Irp (InM-related Protein), Wnt-2b/13, Wnt-3/Int-4, Wnt-3a (R&D sytems), Wnt-4, Wnt-5a, Wnt-5b, Wnt-6 (Kirikoshi H et al 2001 Biochem Biophys Res Com 283 798-805), Wnt-7a (R&D sytems), Wnt-7b, Wnt-8a/8d, Wnt-8b, Wnt-9a/14, Wnt-9b/14b/15, Wnt-IOa, Wnt-10b/12, WnM I, and/or Wnt-16. An overview of human Wnt proteins is provided in “THE WNT FAMILY OF SECRETED PROTEINS”, R&D Systems Catalog, 2004. Further Wnt agonists are or include the R-spondin family of secreted proteins, which is implicated in the activation and regulation of Wnt signaling pathway and which is comprised of 4 members (R-spondin 1 (NU206, Nuvelo, San Carlos, Calif.), R-spondin 2 ((R&D sytems), R-spondin 3, and R-spondin-4), and Norrin (also called Nome Disease Protein or NDP) (R&D sytems), which is a secreted regulatory protein that functions like a Wnt protein in that it binds with high affinity to the Frizzled-4 receptor and induces activation of the Wnt signaling pathway (Kestutis Planutis et al (2007) BMC Cell Biol 8 12). A small-molecule agonist of the Wnt signaling pathway, an aminopyrimidine derivative, was recently identified and is also expressly included as a Wnt agonist (Lm et al (2005) Angew Chem Int Ed Engl 44, 1987-90).

Preferably, in the method for culturing epidermis-derived stem cells according to the present invention, a basal cell culture medium as described in any one of the embodiments herein is employed which further comprises ethanolamine, phospho-ethanolamine and/or transferrin.

The method for culturing epidermis-derived stem cells according to the present invention can in principle be performed for an unlimited time. As mentioned elsewhere herein it is, however, a particular advantage of the method according to the present invention to allow for long-term culturing. Accordingly the method is preferably a method for long-term culturing. In particular, the method is a method for culturing epidermis-derived stem cells, wherein said culturing is performed for at least 360 days, at least 250 days, at least 150 days, at least 100 days, at least 50 days, at least 20 days, at least 14 days, at least 10 days, at least 8 days, at least 5 days, at least 3 days, at least 2 days. Although the method is particularly suited for long-term culturing, in principle in this context lower minimal culturing times are preferred.

Similarly, the method for culturing epidermis-derived stem cells according to the present invention is preferably a method for expanding said epidermis-derived stem cells (in vitro/ex vivo).

Preferably, the method for culturing epidermis-derived stem cells according to the present invention is a method for generating a mixture of cell types comprising epidermis-derived stem cells and at least one differentiated cell type.

Moreover, the method for culturing epidermis-derived stem cells according to the present invention is further a method for enriching said epidermis-derived stem cells provided in said mixture of cell types or said isolated tissue fragment relative to the other cell type(s) comprised in a mixture of cell types or a isolated tissue fragment subjected to the method of culturing according the method provided herein.

Preferably, the method of culturing epidermis-derived stem cells as described in any one of the embodiments herein may further be a method for maintaining the multipotency, proliferative potential and/or capacity to self-renew of epidermis-derived stem cells.

Cell potency is the ability of a cell to give rise to other cell types. In the context of the present invention the term “multipotency” describes the potential of stem cells to differentiate into multiple different, but a limited number of different cell types. In the case of epidermis-derived stem cells, multipotency describes the potential to generate both the differentiated cell types in the interfollicular epidermis as well as in the hair follicles. This property can be, for example, be assessed in full thickness skin reconstitution assays, also referred to as transplantation assays as described in detail further below where the ability of a given epidermis-derived stem cell to generate interfollicular epidermis and/or hair follicles can be assayed. For confirming multipotency of HFSCs and/or interfollicular epidermal stem cells preferably the ability to generate both hair follicles (as detected for example by hair growth) and interfollicular epidermis is measured. For confirming multipotency of epidermis-derived cancer stem cells the ability of these cells to generate a tumor consisting of all differentiated cell types of the tumor of origin is measured.

The capacity of self-renew describes the ability of a stem cell to go through numerous cycles of cell division while maintaining the undifferentiated stem cell state. This property can be assessed by defining the amount of stem cells present in a culture or tissue over a period of time. The amount of epidermis-derived stem cells can, for example, be determined by the FACS analysis as described elsewhere herein.

Stem cells are characterized by their ability to generate a large progeny of differentiated cells. This is in contrast to differentiated cells that can undergo a limited number of cell divisions and thereby produce only limited amount of progeny. This characteristic is referred to as proliferative potential and can be assessed in a colony-forming assay. A colony-forming assay can, for example be conducted as previously described by Jensen and colleagues (Jensen et al., 2010, Nat Protoc 5: 898-911). For example, 2000-4000 cells are plated on 6-well plates containing Mitomycin C-treated feeder cells (J2 fibroblasts). Cultures are incubated at 37° C., 5% CO₂ for 12-14 days during which the medium is replaced every 2 days. Experiments are typically terminated when colonies reach a sufficient size to be visually identified and quantified. Then, colonies are fixed with 4% PFA 10 min and are subsequently stained with 1% crystal violet. Colony number and area are determined using the ImageJ software. Preferably, each condition to be analyzed is performed in triplicates.

In one aspect, the method for culturing epidermis-derived stem cells as described in herein is further a method for amplifying epidermis-derived stem cells, wherein said method further comprises a step of isolating said epidermis-derived stem cells from said cell mixtures after said culturing. In this context the isolation/purification of epidermis-derived stem cells can be achieved by methods known in the art and as described herein elsewhere. In particular, for example, the same methods as used for isolating/purifying/separating epidermis-derived stem cells prior to culturing (starting from a single cell suspension) may be employed. Preferably the step of isolation/purification/separation results in reducing the content of cells other than epidermis-derived stem cells below 1%, below 2%, below 3%, below 5% or below 10% of cells other than epidermis-derived stem cells.

Preferably, the method for culturing epidermis-derived stem cells is employed without employing “feeder cells” or conditioned medium produced by feeder cells. “Feeder cells” or as sometimes interchangeably referred to as “feeders” are cells, typically of fibroblastic origin, which are placed in layer in a cell culture dish and are simultaneously cultured with stem cells. Feeder cells in this context serve as a substrate for the stem cells to grow on. Inter alia, they may secrete a number of essential growth factors that are important for maintaining, for example, the multipotency of the cultured stem cells. Feeder cells can alternatively be cultured to produce a medium termed conditioned medium. Here feeders are cultured for a period of time, typically 2-7 days, during which the cells secrete growth factors and other bioactive compounds into the culture medium. This culture medium, now termed condition medium, can subsequently be transferred to cells to promote their survival and growth. The precise composition of the condition medium is not defined.

The culturing of epidermis-derived stem cells can also be performed with epidermis-derived SCs, preferably hair follicle stem cells or cancer stem cells that are de novo generated ex vivo. Methods for de novo generating epidermis-derived SCs, preferably hair follicle stem cells or cancer stem cells, ex vivo are not known in the art but are also provided herein below. Thus, in the method for culturing epidermis-derived stem cells of the present invention, said epidermis-derived stem cells are preferably epidermis-derived SCs, preferably hair follicle stem cells or cancer stem cells that are de novo generated by the method for ex vivo de novo generation of epidermis-derived SCs, preferably hair follicle stem cells or cancer stem cells, as described in any embodiment of the present invention.

The present invention also provides for a method for expanding epidermis-derived stem cells comprising the step of culturing epidermis-derived stem cells in the presence of a three-dimensional extracellular matrix (3D-ECM) and a basal cell culture medium comprising Epidermal Growth Factor (EGF), a Vascular Endothelial Growth Factor (VEGF), a Fibroblast Growth Factor (FGF) and a Rho-kinase (ROCK) inhibitor.

In the context of the method for expanding epidermis-derived stem cells the term “expanding” means that the number of cells, e.g. of a specific cell type (e.g. epidermis-derived stem cells) increases during the time of in vitro culturing by mitotic cell division. In principle, expansion of the cultivated cells during culturing can be unlimited. Preferably, expansion means to increase the number of epidermis-derived stem cells by at least 1.5-fold, 2-fold, 5-fold, 10-fold, 20-fold, 50-fold, 100-fold or 200-fold during the time of culturing. Culturing in this context relates to maintaining cells in vitro/ex vivo.

The definitions and preferences as regarding the epidermis-derived stem cells as described in the context of the method for culturing epidermis-derived stem cells further below also apply mutatis mutandis for the method for expanding epidermis-derived stem cells. Accordingly, for example, preferably epidermis-derived stem cells are HFSCs, interfollicular epidermal stem cells and/or epidermis-derived CSCs. Even more preferably, epidermis-derived stem cells are HFSCs and/or epidermis-derived CSCs. Most preferably epidermis-derived stem cells are HFSCs.

Similarly, the definitions and preferences as regarding the 3D-ECM/ECM and the basal cell culture medium (in particular also as regarding EGF, VEGF and FGF selection(s)) as described in the context of the method for culturing epidermis-derived stem cells further below also apply mutatis mutandis for the method for expanding epidermis-derived stem cells.

The present invention further relates to a method for ex vivo de novo generation of epidermis-derived stem cells comprising the step of culturing epidermal cells that lack epidermis-derived stem cells intended to be de novo generated in the presence of a three-dimensional extracellular matrix (3D-ECM) as defined mutatis mutandis in any embodiment relating to 3D-ECM in the context of the method of culturing epidermis-derived stem cells elsewhere herein and a basal cell culture medium as defined mutatis mutandis in any embodiment relating to basal cell culture in the context of the method of culturing epidermis-derived stem cells elsewhere herein, wherein said culturing is performed for at least 2 days, at least 5 days, at least 12 days, at least 14 days or at least 360 days. In particular, also the preferences relating to the basal cell culture components and/or the three-dimensional extracellular matrix as described for the method for culturing epidermis-derived stem cells apply mutatis mutandis. Preferably, epidermis-derived stem cells in this context are hair follicle stem cells, interfollicular epidermal stem cells and/or epidermis-derived cancer stem cells. More preferably, epidermis-derived stem cells in this context are hair follicle stem cells and/or epidermis-derived cancer stem cells. Most preferably epidermis-derived stem cells are HFSCs. These preferences refer to both the de novo generated epidermis-derived stem cells and the ones lacking in the epidermal cells used for the de novo generation method.

In principle, a shorter culturing time is preferred. A higher yield of epidermis-derived stem cells can, however, be achieved by culturing said epidermal cells for at least at least 2 days, at least 6 days, at least 12 days, at least 18 days, at least 24 days or at least 30 days. The longer the culturing time, the higher is the yield.

In the context of the method for ex vivo de novo generation of hair follicle stem cells according to the present invention the epidermal cells are preferably selected from a group consisting of the following cell types: epidermal cells of the interfollicular epidermis or of the hair follicle that are depleted of HFSCs as defined by the set of cell surface markers mentioned. Epidermal cells of the interfollicular epidermis are particularly preferred in this context. Preferred is also the usage of epidermal cells originating from a mammal that lack HFSCs, e.g. due to a disease and/or genetic defect.

Particularly in the context of HFSCs, non-HFSCs characterized in that they are CD34⁻α6⁺ are employed.

In the context of the method for ex vivo de novo generation of interfollicular epidermis stem cells according to the present invention the epidermal cells are preferably selected from a group consisting of HFSCs (characterized in that they are CD34+α6⁺) and epidermal cells of the interfollicular epidermis (characterized in that they are CD34⁻α6⁺).

In the context of the method for ex vivo de novo generation of cancer stem cells according to the present invention the epidermal cells are epidermal cells from epidermis-derived tumors (e.g. papilloma and/or carcinoma) that are depleted of CSCs as defined by the set of cell surface markers mentioned above are employed in this context.

Particularly in the context of CSCs, non-CSCs characterized in that they are CD140α⁻, CD31⁻, CD45⁻, EpCAM⁺, CD34⁻α6⁺ are employed.

The present invention also relates to an epidermis-derived stem cell, wherein said epidermis-derived stem cell is obtainable by a method for culturing epidermis-derived stem cells according to the present invention or a method for de novo ex vivo generation of epidermis-derived stem cells. The present invention further relates to a cell mixture comprising said epidermis-derived stem cell. Similarly the present invention relates to a tissue fragment comprising said epidermis-derived stem cell.

Preferably, said epidermis-derived stem cell according to the present invention is a HFSC, an interfollicular epidermis stem cell, or an epidermis-derived CSC (preferably a papilloma-derived or a carcinoma-derived epidermis-derived CSC). Most preferably the epidermis-derived stem cell is a HFSC.

The epidermis-derived stem cell and/or the cell mixture comprising said epidermis-derived stem cell and/or the tissue fragment comprising said epidermis-derived stem cell according to the present invention is preferably characterized by a higher potential of hair formation and/or interfollicular epidermis formation after transplantation. Such higher potential may, for example, be measured by transplantation experiments into immunocompromised hairless mice where maintenance of multipotency can be measured by the ability of the said stem cells to regenerate hair and/or interfollicular epidermis. These type of transplantation assays are also known in the art as “full thickness skin reconstitution assays” as described for example in Jensen et al., 2010, Nat Protoc, 5: 898-911. In this assay epidermis-derived cells containing stem cells (preferentially around 1-5×10⁵ epidermal cells) are mixed with dermal fibroblasts (preferentially around 5×10⁶ fibroblasts) and grafted onto the back of immunocompromised hairless mice. When epidermis-derived stem cells are present in the transplanted cell mixture, they will generate hair follicles that will subsequently grow hair and/or interfollicular epidermis de novo. A higher/increased potential of hair formation can, for example be measured by comparing the amount of hair follicles generated by the cultured and freshly isolated epidermis-derived stem cells. In principle, also the morphology of the grown hair may be compared. A higher/increased potential of interfollicular epidermis formation can, for example, be measured by comparing the area (e.g. in cm²) of newly grown interfollicular epidermis between cultured and freshly isolated epidermis-derived stem cells. Alternatively, also the speed of interfollicular epidermis growth may be compared by comparing the times in which a distinct area of interfollicular epidermis has been generated. Preferably, HFSCs are characterized by a higher/increased potential of hair follicle/hair formation. Similarly, interfollicular epidermal stem cells are preferably characterized by a higher/increased potential of interfollicular epidermis formation. Bona fide multipotent HFSCs and interfollicular epidermis stem cells are capable of generating both hair follicles and interfollicular epidermis. These transplantation experiments can be performed with either mouse or human cells.

Further the present invention also relates to a pharmaceutical composition comprising a epidermis-derived stem cell according to the present invention, a cell mixture according to the present invention, or tissue fragment according to the present invention along with other cell types such as fibroblasts and/or an ECM scaffold.

The present invention further provides an epidermis-derived stem cell according to the present invention, a cell mixture comprising said epidermis-derived stem cell according to the present invention, a tissue fragment comprising said epidermis-derived stem cell or a pharmaceutical composition according to the present invention for use as a medicament. The invention also relates to corresponding method of treatments.

In particular, the invention also relates to an epidermis-derived stem cell according to the present invention, a cell mixture comprising said epidermis-derived stem cell according to the present invention, a tissue fragment comprising said epidermis-derived stem cell or a pharmaceutical composition according to the present invention for use in tissue transplantation. Preferably, the tissue in this context is skin or even more preferably skin with hairs. Most preferably, said epidermis-derived stem cell is a hair follicle stem cell and said tissue transplantation reconstitutes hair growth.

Preferably, an epidermis-derived stem cell according to the present invention, a cell mixture comprising said epidermis-derived stem cell according to the present invention, a tissue fragment comprising said epidermis-derived stem cell or a pharmaceutical composition according to the present invention for use in treatment of dermal burn, treatment of conditions where areas of skin have been removed due to surgical operation, biopsy or trauma, chronic wounds, baldness is provided. Preferably, in this context baldness is selected from the group consisting of male pattern baldness, nutrition-induced baldness, infection-induced baldness, drug-induced baldness, traction alopecia or other trauma-induced baldness, pregnancy-induced baldness, alopecia areata and/or alopecia mucinosa.

The present invention also relates to a method of treating baldness. Also here baldness is preferably selected from the group consisting of male pattern baldness, nutrition-induced baldness, infection-induced baldness, drug-induced baldness, traction alopecia or other trauma-induced baldness, pregnancy-induced baldness, alopecia areata and/or alopecia mucinosa. Preferably, the method of treating baldness is exclusively used for cosmetic purposes.

The present invention further provides uses of an epidermis-derived stem cell, a cell mixtures comprising said epidermis-derived stem cell or a tissue fragment comprising said epidermis-derived stem cells according to the present invention.

The present invention also relates to an epidermis-derived stem cell, a cell mixtures comprising said epidermis-derived stem cell or a tissue fragment comprising said epidermis-derived stem cells according to the present invention for in vitro tissue production. Preferably the tissues generated are skin or skin comprising hair follicle (preferably also hairs). Said in vitro tissue production may, for example be achieved by isolating epidermal cells from a tissue biopsy. These cells can be cultured using the method of the invention allowing expansion of multipotent epidermis-derived stem cells or alternatively generation of hair follicle stem cells de novo from hairless epidermis and their subsequent expansion. This method would thus allow generation of epidermis-derived stem cells in large quantities for transplantation, or alternatively for generation of epidermal organs in vitro (so called organotypic cultures; Oh et al., 2013, J Invest Dermatol 133: e14 doi:10.1038/jid.2013.387) that can be grafted or transplanted to recipients at a later time point.

In another aspect, the present invention relates to an epidermis-derived stem cell, a cell mixtures comprising said epidermis-derived stem cell or a tissue fragment comprising said epidermis-derived stem cells according the present invention for in vitro drug discovery screening.

In the context of the present invention the term “in vitro drug discovery screening” refers to a method which comprises the step of contacting said epidermis-derived stem cells with one or more molecules that are intended as a medicament. In particular, such a method may further comprise a step that allows for determining whether the intended medical effect is achieved. Preferably, said drug discovery screening is performed to identify molecules/compounds that affect stem cell function, quiescence, growth and/or differentiation. Similarly, said drug discovery screening is performed to identify molecules/compounds that modulate hair growth. Modulating hair growth includes increasing or decreasing hair growth upon application of said molecule/compound (e.g. systematically or locally) to an organism (preferably a mammal, most preferably a human). An increase or decrease in hair growth may, for example, be an increase in the number of hairs of at least 2%, at least 4%, at least 6%, at least 10% or at least 20%, whereas a decrease in hair growth may, for example, be an decrease in the number of hairs of at least 2%, at least 4%, at least 6%, at least 10% or at least 20%.

The present invention further relates to a method for culturing epidermis-derived stem cells, wherein said method additionally comprises a step of in vitro generating a tissue from the cultured epidermis-derived stem cells. Similarly, the invention also provides a method for ex vivo de novo generation of epidermis-derived stem cells according to the present invention, wherein said method additionally comprises a step of in vitro generating a tissue from the cultured epidermis-derived stem cells.

Further, the present invention provides a method for culturing epidermis-derived stem cells, wherein said method additionally comprises a step of incubating said cultured epidermis-derived stem cells with a drug candidate. Preferably, said drug candidate is a molecule as described above for the use of an epidermis-derived stem cell according to the present invention for in vitro drug discovery screening. Most preferably said drug candidate modulates stem cell function, quiescence, growth and/or differentiation and/or hair growth. Similarly, the invention also provides a method for ex vivo de novo generation of epidermis-derived stem cells according to the present invention, wherein said method additionally comprises a step of incubating said cultured epidermis-derived stem cells with a drug candidate. In this context the same preferences for the drug candidate as described above for the method of culturing epidermis-derived stem cells apply.

The present invention also relates to a cell culture medium as defined mutatis mutandis in the sections describing a method for culturing epidermis-derived stem cells or a method for ex vivo de novo generation of epidermis-derived stem cells. Accordingly, the present invention, for example, relates to a cell culture medium which is a basal cell culture medium that further comprises:
Epidermal Growth Factor (EGF); and/or
a Vascular Endothelial Growth Factor (VEGF); and/or
a Fibroblast Growth Factor (FGF);
and further a ROCK (Rho-kinase) inhibitor.

Similarly, the invention provides a cell culture medium which is a basal cell culture medium that further comprises:
Epidermal Growth Factor (EGF); and
a Vascular Endothelial Growth Factor (VEGF); and/or
a Fibroblast Growth Factor (FGF);
and further a ROCK (Rho-kinase) inhibitor.

The present invention also relates to a cell culture medium which is a basal cell culture medium that further comprises:
Epidermal Growth Factor (EGF); and
a Vascular Endothelial Growth Factor (VEGF); and
a Fibroblast Growth Factor (FGF);
and further a ROCK (Rho-kinase) inhibitor.

A preferred cell culture medium is a basal cell culture medium that comprises:
Epidermal Growth Factor (EGF); and
a Vascular Endothelial Growth Factor (VEGF); and
a Fibroblast Growth Factor (FGF);
and further a ROCK (Rho-kinase) inhibitor,
wherein said basal cell culture medium further comprises a SHH inhibitor.

The selection of components of the basal cell culture medium, the concentration of components that are added to the basal cell culture medium have been described in the context of the method for culturing epidermis-derived stem cells and apply here mutatis mutandis.

The present invention also relates to uses of a method for culturing epidermis-derived stem cells and/or a method for ex vivo de novo generation of epidermis-derived stem cells according to the present invention. Everything mentioned in the context of the methods of the present invention applies here mutatis mutandis.

In particular, the use of a method for culturing epidermis-derived stem cells and/or a method for ex vivo de novo generation of epidermis-derived stem cells according to the present invention for expanding/growing said epidermis-derived stem cells is envisaged.

Expanding/growing means that the number of cells is increased during the time of in vitro culturing by mitotic cell division. In principle, expansion of the cultivated cells during culturing can be unlimited. Preferably, expansion means to increase the number of epidermis-derived stem cells by at least 1.5-fold, 2-fold, 5-fold, 10-fold, 20-fold, 50-fold, 100-fold or 200-fold during the time of culturing.

Furthermore, the use of a method for culturing epidermis-derived stem cells and/or a method for ex vivo de novo generation of epidermis-derived stem cells according to the present invention for generating a mixture of cell types comprising said epidermis-derived stem cells and at least one differentiated epidermal cell type is envisaged. Thereby a differentiated epidermal cell type can be a differentiated epidermal cell type as defines elsewhere herein.

Another use of method for culturing epidermis-derived stem cells and/or a method for ex vivo de novo generation of epidermis-derived stem cells according to the present invention is the use for enriching said epidermis-derived stem cells provided in said mixture of cell types or said isolated tissue fragment relative to the other cell type(s) comprised in said mixture of cell types or said isolated tissue fragment.

Further the use of method for culturing epidermis-derived stem cells and/or a method for ex vivo de novo generation of epidermis-derived stem cells according to the present invention for maintaining multipotency of said epidermis-derived stem cells is provided in the present invention.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The present invention, inter alia, relates to the following items:
1. A method for culturing epidermis-derived stem cells comprising the step of culturing epidermis-derived stem cells in the presence of a three-dimensional extracellular matrix (3D-ECM) and a basal cell culture medium comprising:
   Epidermal Growth Factor (EGF); and/or
   a Vascular Endothelial Growth Factor (VEGF); and/or
   a Fibroblast Growth Factor (FGF);
   and further a Rho-kinase (ROCK) inhibitor.
2. The method of item 1, wherein said basal cell culture medium comprises said EGF, said VEGF, said FGF and said ROCK inhibitor.
3. The method of item 1 or 2, wherein said Vascular Endothelial Growth Factor (VEGF) is selected from the group consisting of VEGF-121, VEGF-120, VEGF-145, VEGF-165, VEGF-164, VEGF-183, VEGF-188, VEGF-189 and VEGF-206.

Under item 3, VEGF is preferably selected from the group consisting of VEGF-164, VEGF-165, VEGF-121, VEGF-120 and VEGF-188. More preferably, VEGF is selected from the group consisting of VEGF-164, VEGF-165 and VEGF-121. Most preferably, VEGF is selected from the group consisting of VEGF-164 and VEGF-121.
4. The method of any one of items 1 to 3, wherein said Fibroblast Growth Factor (FGF) is selected from the group consisting of: FGF-1, FGF-2, FGF-3, FGF-4, FGF-5, FGF-6, FGF-7, FGF-10, FGF-22, FGF-8, FGF-17, FGF-18, FGF-24, FGF-9, FGF-16, FGF-20, FGF-11, FGF-12, FGF-13, FGF-14, FGF-19, FGF-21, and FGF-23.

Under item 4, FGF is preferably selected from the group consisting of FGF-2, FGF-7, FGF-10 and FGF-18. Even more preferably FGF is from the group consisting of FGF-2 and FGF-18.
5. The method of any one of items 1 to 4, wherein said ROCK inhibitor is selected from the group consisting of (R)-(+)-trans-4-(1-aminoethyl)-N-(4-Pyridyl)cyclohexanecarboxamide dihydrochloride monohydrate (Y-27632), 5-(1,4-diazepan-1-ylsulfonyl) isoquinoline (fasudil or HA 1077), and (S)-(+)-2-methyl-1-[(4-methyl-5-isoquinolinyl)sulfonyl]-hexahydro-1H-1,4 diazepine dihydrochloride (H-1152).

Under item 5 said ROCK inhibitor is preferably selected from the group consisting of (S)-(+)-2-methyl-1-[(4-methyl-5-isoquinolinyl)sulfonyl]-hexahydro-1H-1,4 diazepine dihydrochloride (H-1152) and (R)-(+)-trans-4-(1-aminoethyl)-N-(4-Pyridyl)cyclohexanecarboxamide dihydrochloride monohydrate (Y-27632). Most preferably the ROCK inhibitor is (R)-(+)-trans-4-(1-aminoethyl)-N-(4-Pyridyl)cyclohexanecarboxamide dihydrochloride monohydrate (Y-27632).
6. The method of any one of items 1 to 5, wherein said epidermis-derived stem cells are human.
7. The method of any one of items 1 to 6, wherein said EGF is human EGF and/or said VEGF is a human VEGF and/or said FGF is a human FGF.
8. The method of any one of items 1 to 7, wherein said ROCK inhibitor is an inhibitor of a human ROCK.
9. The method of any one of items 1 to 8, wherein said EGF is comprised in the basal cell culture medium at a concentration of between 2 ng/ml and 500 ng/ml, preferably of between 5 ng/ml and 100 ng/ml or most preferably of between 10 ng/ml and 50 ng/ml.
10. The method of any one of items 1 to 9, wherein said VEGF is comprised in the basal cell culture medium at a concentration of between 2 ng/ml and 500 ng/ml, preferably of between 5 ng/ml and 100 ng/ml or most preferably of between 10 ng/ml and 50 ng/ml.
11. The method of any one of items 1 to 10, wherein said FGF is comprised in the basal cell culture medium at a concentration of between 2 ng/ml and 500 ng/ml, preferably of between 5 ng/ml and 100 ng/ml or most preferably of between 10 ng/ml and 50 ng/ml.
12. The method of any one of items 1 to 11, wherein said ROCK inhibitor is comprised in the basal cell culture medium at a concentration of between 2 μM and 20 μM, preferably of between 3 μM and 15 μM or most preferably of between 5 μM and 10 μM.
13. The method of any one of items 1 to 12, wherein said basal cell culture medium further comprises a Sonic Hedgehog (SHH) inhibitor.
14. The method of item 13, wherein said SHH inhibitor is selected from the group consisting of (2′R,3S,3′R,3′aS,6'S,6aS,6bS,7′aR,11aS,11bR)-1,2,3,3′a,4,4′,5′,6,6′,6a,6b,7,7′,7′a,8,11,11a,11b-Octadecahydro-3′,6′,10,11b-tetramethylspiro[9H-benzo[a]fluorene-9,2′(3′H)-furo[3,2-b]pyridin]-3-ol (Cyclopamine), 2-chloro-N-(4-chloro-3-pyridin-2-ylphenyl)-4-methylsulfonylbenzamide (HhAntag691), N-((2S,3R,3aS,3′R,4a′R,6S,6a′R,6b'S,7aR,12a'S,12b'S)-3,6,11′,12b′-tetramethyl-2′,3a,3′,4,4′,4a′,5,5′,6,6′,6a′,6b′,7,7a,7′,8′,10′,12′,12a′,12b′-icosahydro-1′H,3H-spiro[furo[3,2-b]pyridine-2,9′-naphtho[2,1-a]azulen]-3′-yl)methanesulfonamide (IPI-926), N-[6-[(2S,6R)-2,6-dimethylmorpholin-4-yl]pyridin-3-yl]-2-methyl-3-[4-(trifluoromethoxy)phenypenzamide (LDE225) and N-R4-chlorophenyl)methyl]-2-[(2R,6S,8E)-5,12-dioxo-2-phenyl-1-oxa-4-azacyclododec-8-en-6-yl]acetamide (Robotnikinin).
15. The method of item 13 or 14, wherein said SHH inhibitor is comprised in the basal cell culture medium at a concentration of between 1 μM and 20 μM, preferably between 5 μM and 15 μM, most preferably between 5 μM and 10 μM.
16. The method of any one of items 1 to 15, wherein said basal culture medium does not comprise a Bone Morphogenetic Protein (BMP) inhibitor.
17. The method of item 16, wherein the BMP inhibitor is selected from the group consisting of Noggin, 6-[4-(2-piperid in-1-ylethoxy)phenyl]-3-pyridin-4-ylpyrazolo[1,5-a]pyrimidine; dihydrochloride (Dorsomorphin), 3-[6-amino-5-(3,4,5-trimethoxyphenyl)pyridin-3-yl]phenol (K02288), 4-[6-(4-propan-2-yloxyphenyl)pyrazolo[1,5-a]pyrimidin-3-yl]quinoline (DMH.1), 2-(hydroxymethyl)-6-[3-(hydroxymethyl)phenoxy]oxane-3,4,5-triol (ML 347), 4-[6-(4-piperazin-1-ylphenyl)pyrazolo[1,5-a]pyrimidin-3-yl]quinoline (LDN-193189).
18. The method of any one of items 1 to 17, wherein said basal cell culture medium further comprises ethanolamine, phospho-ethanolamine and/or transferrin.
19. The method of any one of items 1 to 18, wherein said basal cell culture medium further comprises L-glutamine, insulin, hydrocortisone, penicillin, streptomycin, calcium-depleted fetal calf serum and/or calcium-depleted fetal bovine serum.
20. The method of any one of items 1 to 19, wherein said method is further a method for expanding said epidermis-derived stem cells.
21. The method of any one of items 1 to 20, wherein said culturing of epidermis-derived stem cells is performed for at least 360 days, at least 250 days, at least 100 days, at least 50 days, at least 20 days, at least 14 days, at least 10 days, at least 8 days, at least 5 days, at least 3 days or at least 2 days.
22. The method of any one of items 1 to 21, wherein said epidermis-derived stem cells are hair follicle stem cells (HFSCs) or cancer stem cells (CSCs).
23. The method of item 22, wherein said cancer stem cells are papilloma-derived or carcinoma-derived stem cells.
24. The method of any one of items 1 to 23, wherein said epidermis-derived stem cells:
   are comprised in a mixture of cell types, which further comprises at least one differentiated epidermal cell type;
   are comprised in an isolated tissue fragment; or
   are isolated.
25. The method of item 24, wherein:
   said mixture of cell types comprises isolated keratinocytes, epidermal papilloma cells or epidermal carcinoma cells; or
   said isolated tissue is skin.
26. The method of any one of items 1 to 25, which is a method for generating a mixture of cell types comprising epidermis-derived stem cells and at least one differentiated cell type.
27. The method of item 25 or 26, wherein said method is a method for enriching said epidermis-derived stem cells provided in said mixture of cell types or said isolated tissue fragment relative to the other cell type(s) comprised in said mixture of cell types or said isolated tissue fragment.
28. The method of any one of items 1 to 27, wherein said epidermis-derived stem cells are maintaining their multipotency, proliferative potential and/or capacity to self-renew.
29. The method of any one of items 1 to 28, wherein said 3D-ECM is a synthetic hydrogel 3D-ECM or a naturally occurring 3D-ECM.
30. The method of any one of items 1 to 29, wherein said 3D-ECM comprises one or more component selected from the group consisting of collagen type I, collagen type II, collagen type III, collagen type IV, laminin-111, laminin-211, laminin-121, laminin-221, laminin-332, laminin-3B32, laminin-311, laminin-321, laminin-411, laminin-421, laminin-511, laminin-521, laminin-213, laminin-423, laminin-522, laminin-523, entactin, perlecan, vitronectin and heparan sulfate proteoglycan.
31. The method of any one of items 1 to 30, wherein said 3D-ECM comprises at least one ECM glycoprotein.
32. The method of any one of items 1 to 31, wherein said 3D-ECM comprises at least two distinct ECM glycoproteins.
33. The method of any one of items 1 to 32, wherein said 3D-ECM comprises a laminin, a collagen and an entactin.
34. The method of item 33, wherein said laminin is laminin-511 or laminin-332.
35. The method of item 33 or 34, wherein said collagen is collagen IV.
36. The method of any one of items 1 to 35, wherein said 3D-ECM comprises:
   laminin-511, collagen IV and an entactin; or
   laminin-332, collagen IV and an entactin.
37. The method of any one of items 30 or 33 to 36, wherein said entactin is entactin-1 or entactin-2 (also known as nidogen 1 or nidogen 2), respectively.
38. The method of any one of items 1 to 37, wherein said 3D-ECM is generated from a soluble form of basement membrane purified from Engelbreth-Holm-Swarm (EHS) tumor.

Under item 38, the ECM is a soluble form of basement membrane purified from Engelbreth-Holm-Swarm (EHS) tumor. In a solidified form it forms a 3D-ECM.
39. The method of any one of items 1 to 38, wherein said ECM comprises or consists of 60% to 85% laminin, 5 to 30% collagen IV, 1 to 10% entactin, and 1 to 10% heparan sulfate proteoglycan.
40. The method of any one of items 1 to 39, wherein said method further comprises a step of isolating said epidermis-derived stem cells from said cell mixtures after said culturing.
41. The method of any one of items 1 to 40, wherein said method does not employ feeder cells.
42. The method of any one of items 1 to 41, wherein said epidermis-derived stem cells are de novo generated ex viva
43. A method for ex vivo de novo generation of epidermis-derived stem cells comprising the step of culturing epidermal cells lacking epidermis-derived stem cells in the presence of an three-dimensional extracellular matrix (3D-ECM) as defined by any one of items 1 and 29 to 39 and a basal cell culture medium as defined in any one of items 1 to 19 for at least 2 days, at least 4 days, at least 6 days, at least 8 days, at least 10 days, at least 12 days, at least 14 days, at least 16 days, at least 18 days, at least 20 days, at least 100 days, at least 200 days, at least 360 days.

Under item 43 the method is preferably performed at least between 4 days and 30 days, most preferred between 8 days and 14 days

Under item 43 epidermis-derived stem cells (referring to both the de novo generated and the lacking epidermis-derived stem cells) are preferably HFSCs and/or epidermis-derived CSCs, most preferably HFSCs.
44. The method of item 43, wherein
   said de novo generated epidermis-derived stem cells are HFSCs and the epidermal cells comprise or consist of: epidermis-derived stem cells other than HFSCs, differentiated epidermal cells or mixtures with all possible combinations of said stem cells and differentiated cells;
   said de novo generated epidermis-derived stem cells are epidermal stem cells other than HFSCs and the epidermal cells comprise or consist of: HFSCs, differentiated epidermal cells or mixtures with all possible combinations of said stem cells and differentiated cells; or
   said de novo generated epidermis-derived stem cells are CSCs and the epidermal cells comprise or consist of: epidermal cells from premalignant, inflamed or otherwise diseased skin, epidermal cells from epidermal tumor tissue, or mixtures with all possible combinations of said stem cells, differentiated cells, premalignant cells and/or tumor cells.
45. An epidermis-derived stem cell that is obtainable by a method as defined in any one of the items 1 to 44.
46. A cell mixture comprising said epidermis-derived stem cell of item 45.
47. A tissue fragment comprising said epidermis-derived stem cell of item 45.
48. A pharmaceutical composition comprising an epidermis-derived stem cell of item 45, a cell mixture of item 46, or a tissue fragment of item 47.
49. The epidermis-derived stem cell of item 45, a cell mixture of item 46, or a tissue fragment of item 47 or the pharmaceutical composition of item 48 for use as a medicament.
50. The epidermis-derived stem cell of item 45, a cell mixture of item 46, or a tissue fragment of item 47 or the pharmaceutical composition of item 48 for use in tissue transplantation.
51. The epidermis-derived stem cell of item 45, a cell mixture of item 46, or a tissue fragment of item 47 or the pharmaceutical composition of item 48 for use in treatment of conditions where areas of skin have been removed due to surgical operation, biopsy, burn and/or trauma, and/or in treatment of conditions where the regenerative capacity of the skin is compromised such as chronic wounds and/or baldness
52. The epidermis-derived stem cell, the cell mixture, the tissue fragment or the pharmaceutical composition according to item 51 for the use according to item 51, wherein baldness is selected from the group of male pattern baldness, nutrition-induced baldness, infection-induced baldness, drug-induced baldness, traction alopecia or other trauma-induced baldness, pregnancy-induced baldness, alopecia areata and alopecia mucinosa.
53. The epidermis-derived stem cell, the cell mixture, the tissue fragment or the pharmaceutical composition according to item 51 for the use according to item 51, wherein said epidermis-derived stem cell is a hair follicle stem cell and said tissue transplantation reconstitutes hair growth.
54. Use of the epidermis-derived stem cell of item 45, a cell mixture of item 46, or a tissue fragment of item 47 for in vitro tissue production.
55. Use of the epidermis-derived stem cell of item 45, a cell mixture of item 46, or a tissue fragment of item 47 for in vitro drug discovery screening.
56. The use of item 55, wherein said drug discovery screening is performed to identify compounds that affect stem cell function, quiescence, growth and/or differentiation.
57. The use of item 55 or 56, wherein said drug discovery screening is performed to identify compounds that modulate hair growth.
58. The method of any one of items 1 to 44, wherein said method additionally comprises a step of generating a tissue in vitro from the cultured epidermis-derived stem cells of any one of items 1 to 44.
59. The method of any one of items 1 to 44 or 58, wherein said method additionally comprises a step of incubating said cultured epidermis-derived stem cells of any one of items 1 to 44 or said tissue of item 58 with a drug candidate.
60. The method of item 59, wherein said drug candidate modulates stem cell function, quiescence, growth and/or differentiation and/or hair growth.
61. A cell culture medium as defined in any one of items 1 to 19.
62. Use of a method as defined in any one of items 1 to 44 or 58 to 60 for expanding said epidermis-derived stem cells.
63. Use of a method as defined in any one of items 1 to 44 or 58 to 60 for generating a mixture of cell types comprising said epidermis-derived stem cells and at least one differentiated epidermal cell type.
64. Use of a method as defined in any one of items 1 to 44 or 58 to 60 for enriching said epidermis-derived stem cells provided in said mixture of cell types or said isolated tissue fragment relative to the other cell type(s) comprised in said mixture of cell types or said isolated tissue fragment.
65. Use of a method as defined in any one of items 1 to 44 or 58 to 60 for maintaining multipotency of said epidermis-derived stem cells.

The present invention is further described by reference to the following non-limiting figures and examples.

The Figures show:

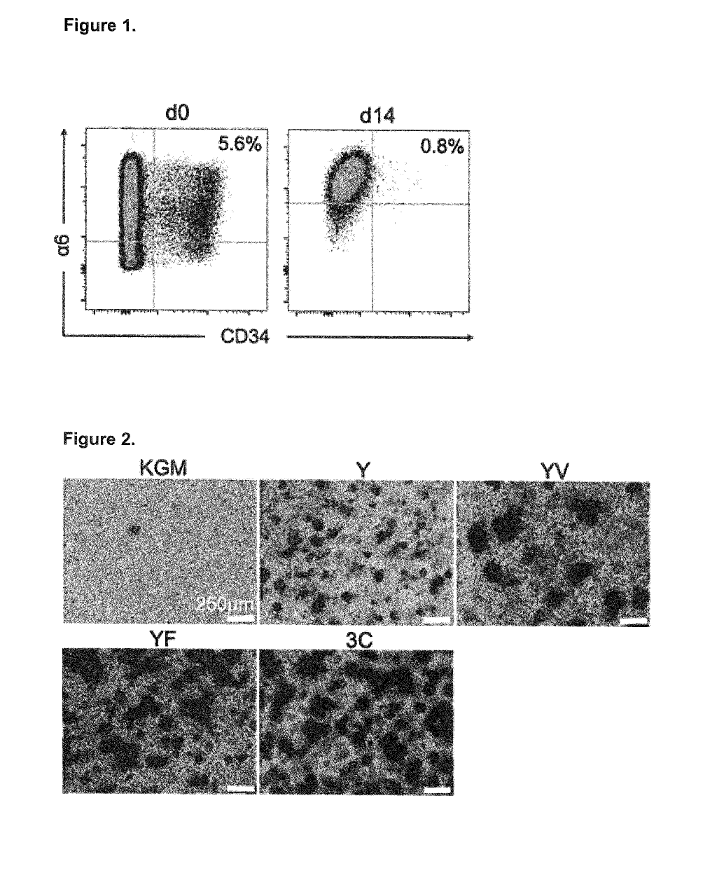

FIG. 1: Standard keratinocyte culture conditions do not support growth of CD34⁺α6⁺ HFSCs.

FACS plots of freshly isolated mouse keratinocytes (day 0; d0) and keratinocytes cultured under standard 2D culture conditions for 2 weeks (day 14; d14). Single cell suspensions were stained for the stem cell markers α6 integrin and CD34. Gates were drawn according to the respective unstained and isotype-stained controls. Percentages are indicated per quadrant.

FIG. 2: Cell growth under different culture conditions

Representative bright field images of epidermal cells grown in the indicated growth media in the presence of 3D-ECM (scale bars 250 μm). Y: EGF+Y27632; YV: Y27632+EGF+VEGF; YF: Y27632+EGF+FGF-2; 3C: Y27632+EGF+VEGF+FGF-2.

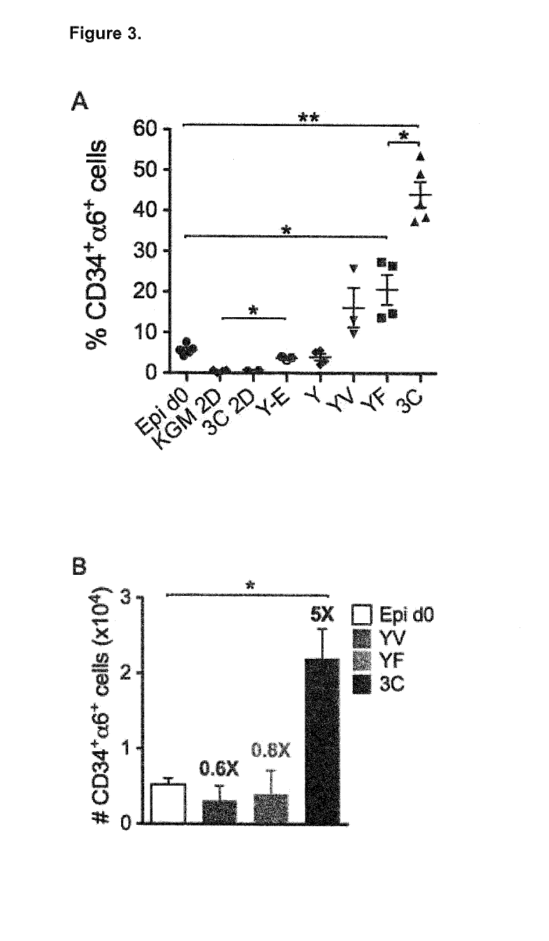

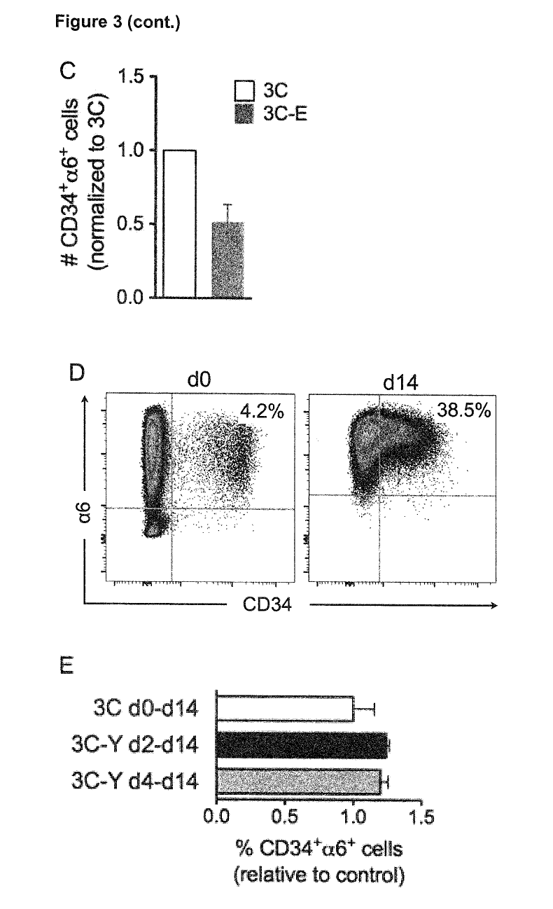

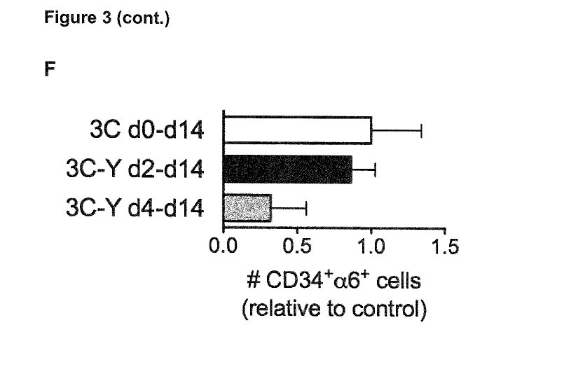

FIG. 3: CD34⁺α6⁺ HFSC enrichment under different culture conditions

A. Percentage of CD34⁺α6⁺ cells from day 14 (d14) HFSC cultures in different growth media. CD34⁺α6⁺ cells were quantified by flow cytometry (mean±SEM, n=4-5; *p≥0.05; **p≥0.01 Mann-Whitney U test). Epi d0: Freshly isolated epidermal cells, KGM 2D: basal medium in 2D; 3C 2D: Y27632+EGF+VEGF+FGF-2 in 2D; Y-E: Y27632 without EGF in the basal medium in 3D-ECM; Y: EGF+Y27632 in 3D-ECM, YV: Y27632+EGF+VEGF in 3D-ECM; YF: Y27632+EGF+FGF-2 in 3D-ECM; 3C: Y27632+EGF+VEGF+FGF-2 in 3D-ECM.

B. Absolute numbers of CD34⁺α6⁺ cells from day 14 (d14) HFSC cultures in different growth media. CD34⁺α6⁺ cells were quantified by flow cytometry. Data are plotted as fold enrichment over freshly isolated cells (mean±SEM, n=4-5; *p≥0.05; **p≥0.01 Mann-Whitney U test). Y: Y27632+EGF; YV: Y27632+EGF+VEGF; YF: Y27632+EGF+FGF-2; 3C: Y27632+EGF+VEGF+FGF-2. All conditions are in 3D-ECM.

C. Normalized numbers of CD34⁺α6⁺ cells from day 14 HFSC cultures in 3C media with (3C) and without (3C-E) EGF. CD34⁺α6⁺ cells were quantified by flow cytometry. Data are normalized to 3C in each of the three independent experiments shown. ±SEM, n=3. 3C: Y27632+EGF+VEGF-164+FGF-2; 3C-E: Y27632+VEGF-164+FGF-2 without EGF in basal medium. All conditions are in 3D-ECM.

D. Representative FACS plots of freshly isolated (day 0; d0) and keratinocytes cultured in 3C conditions for 2 weeks (day 14; d14). Gates were drawn according to the respective unstained and isotype-stained controls. Percentages are indicated per quadrant.

E. Normalized percentages of CD34⁺α6⁺ cells from day 14 HFSC cultures grown under 3C conditions for the indicated times. CD34⁺α6⁺ cells were quantified by flow cytometry. For 3C-Y conditions cells were grown from day 0 to either day 2 or day 4 in 3C conditions and then the medium was exchanged to a 3C medium lacking Y27632 (3C-Y). Data are normalized to 3C conditions in each of the two independent experiments shown. ±SD, n=2. 3C: Y27632+VEGF-164+FGF-2; 3C-Y: VEGF-164+FGF-2; all in basal medium. All conditions are in 3D-ECM. d=days.

F. Normalized absolute numbers of CD34⁺α6⁺ cells from day 14 HFSC cultures grown under 3C conditions for the indicated times. CD34⁺α6⁺ cells were quantified by flow cytometry. For 3C-Y conditions cells were grown from day 0 to either day 2 or day 4 in 3C conditions and then the medium was exchanged to a 3C medium lacking Y27632 (3C-Y). Data are normalized to 3C conditions in each of the two independent experiments shown. ±SD, n=2. 3C: Y27632+VEGF-164+FGF-2; 3C-Y: VEGF-164+FGF-2; all in basal medium. All conditions are in 3D-ECM. d=days.

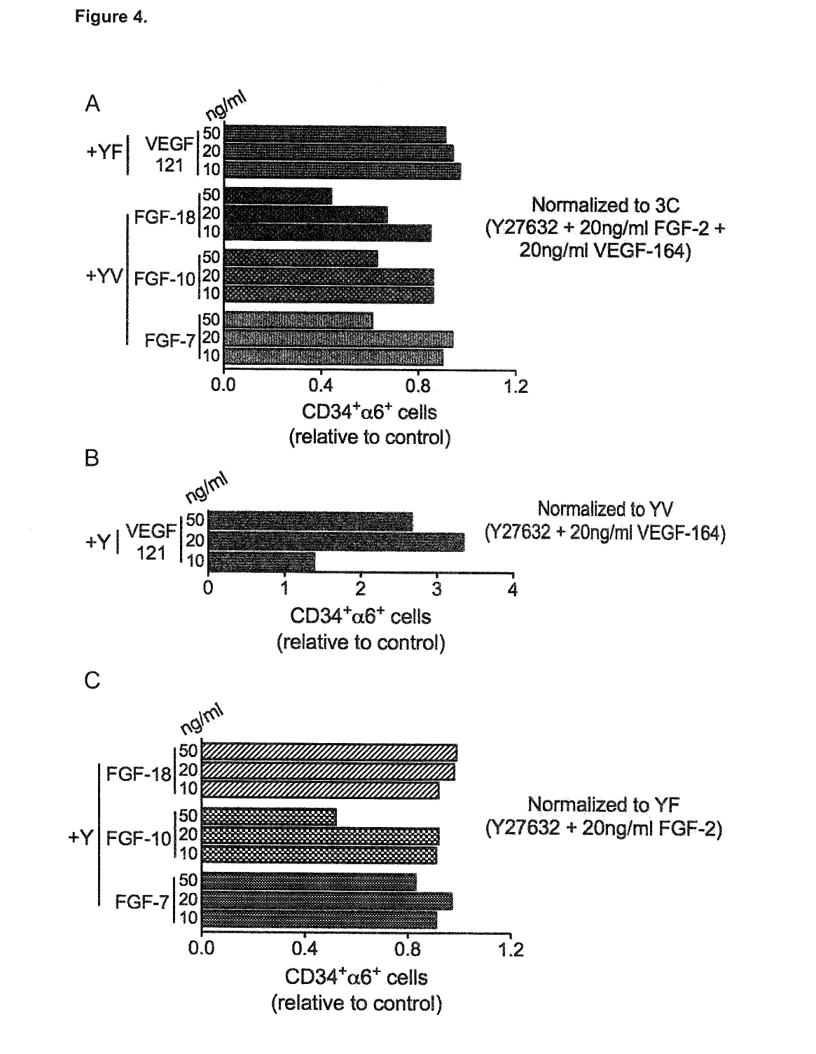

FIG. 4: CD34⁺α6⁺ HFSC enrichment with different growth factor combinations

CD34⁺α6⁺ cells were quantified by flow cytometry from day 14 Matrigel cultures containing various growth factor combinations. Normalized CD34⁺α6⁺ content of cultures is shown. A. Direct comparison of different VEGF and FGF isoforms in 3C conditions (3D-ECM; basal cell culture medium comprising Y27632+EGF+FGF+VEGF). VEGF-121 performs equally well as VEGF-164 (set to 1). FGF-12, FGF-10, and FGF-7 perform equally well as FGF-2 (set to 1). B. Direct comparison of VEGF-121 and VEGF-164 in YV conditions (3D-ECM; basal cell culture medium comprising Y27632+EGF+VEGF). VEGF-121 performs slightly better than VEGF-164 (set to 1). C. Direct comparison of different FGF isoforms in YF conditions (3D-ECM; basal cell culture medium comprising Y27632+EGF+FGF). FGF-12, FGF-10, and FGF-7 perform equally well as FGF-2 (set to 1).

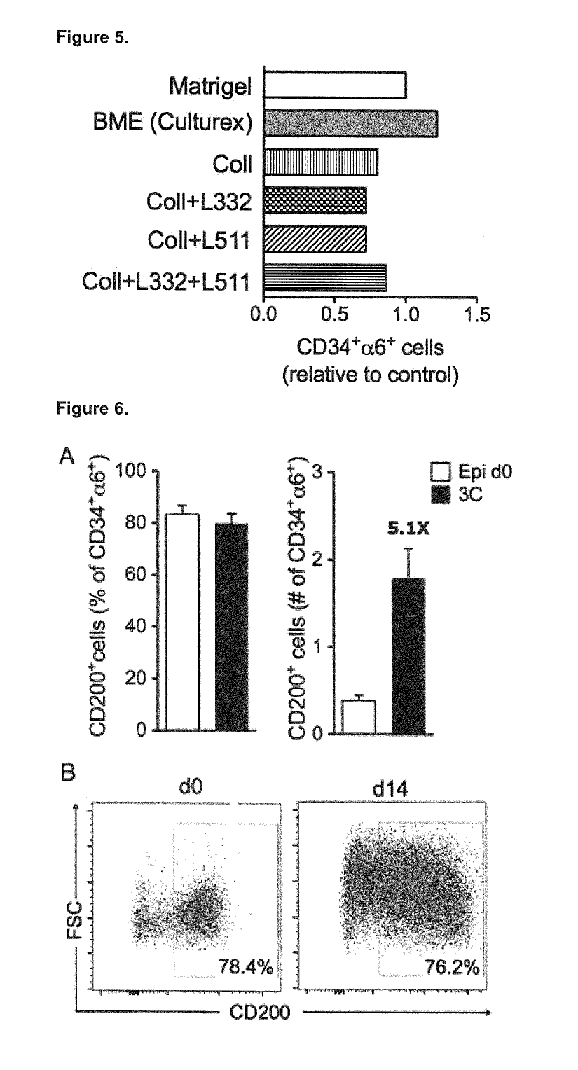

FIG. 5: CD34⁺α6⁺ HFSC enrichment in 3D-ECMs with different compositions

CD34⁺α6⁺ cells were quantified by flow cytometry from day 14 3C cultures in various ECM component-containing 3D-ECM gels. CD34⁺α6⁺ content normalized to 3C Matrigel cultures is shown. Col I: Collagen type I; L332: laminin 332; L511: laminin 511; BME: basement membrane extract.

FIG. 6: 3C cultures enrich for CD200⁺ cells

A. Percentage of CD200⁺ cells in day 14 (d14) HFSC cultures. CD200⁺ cells were quantified by flow cytometry. Fold enrichment is shown over freshly isolated cells (mean±SEM, n=3).

B. Representative FACS plots of freshly isolated (day0) and keratinocytes cultured in 3C conditions for 2 weeks (day 14; d14). Gates were drawn according to the respective unstained and isotype-stained controls. Percentage of CD200⁺ from CD34⁺α6⁺ cells is indicated per quadrant.

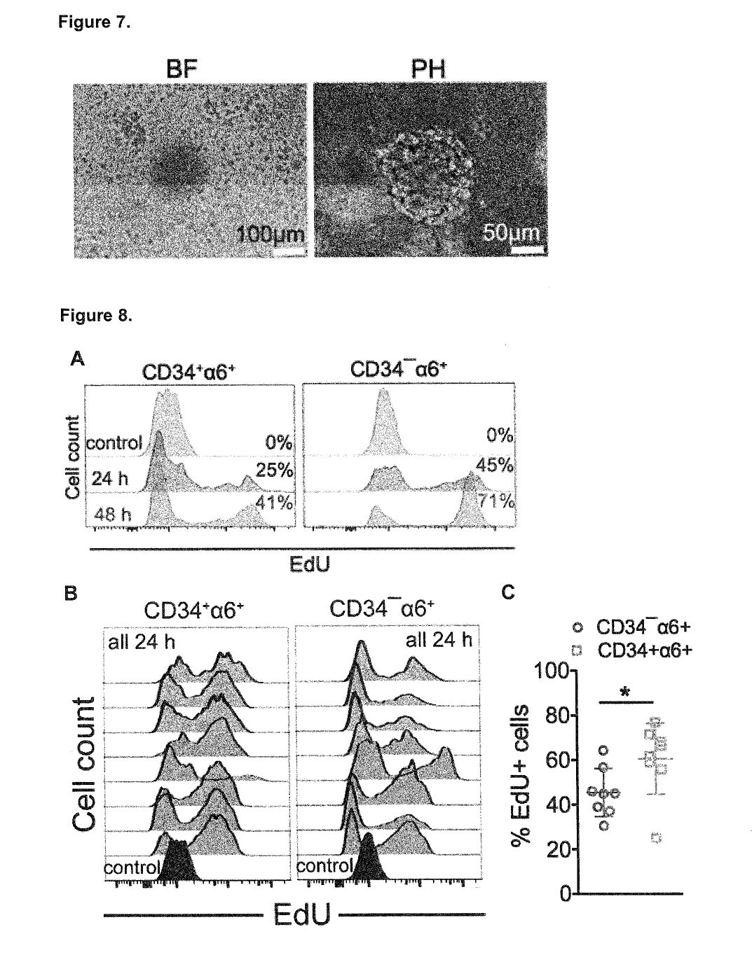

FIG. 7: Morphology of cells cultured under 3C conditions

Representative image of cell clusters formed in 3C cultures of HFSCs. Note that no lumen is observed. BF: bright field; PH: phase contrast. Scale bars 100 μm and 50 μm (right and left panel, respectively).

FIG. 8: Proliferation of cells cultured under 3C conditions

A. FACS histograms of day 14 (d14) 3C cultures of HFSCs labeled with EdU for 24 h (day 13-day 14) and 48 h (day 12-day 14). Percentage of EdU+ cells in CD34⁺α6⁺ and CD34⁻α6⁺ cells is indicated. Cells without EdU are shown as control. The Figure shows a single experiment.

B. FACS histograms of eight different 3C cultures of HFSCs labeled with EdU for 24 h (between days 10-11 and 12-13). Percentage of EdU+ cells in CD34⁺α6⁺ and CD34⁻α6⁺ cells were quantified by flow cytometry. Cells without EdU are shown as control in black color.

C. Pooled results from individual experiments shown in panels A and B (mean±SEM, n=8; *p≥0.05 Mann-Whitney U test)

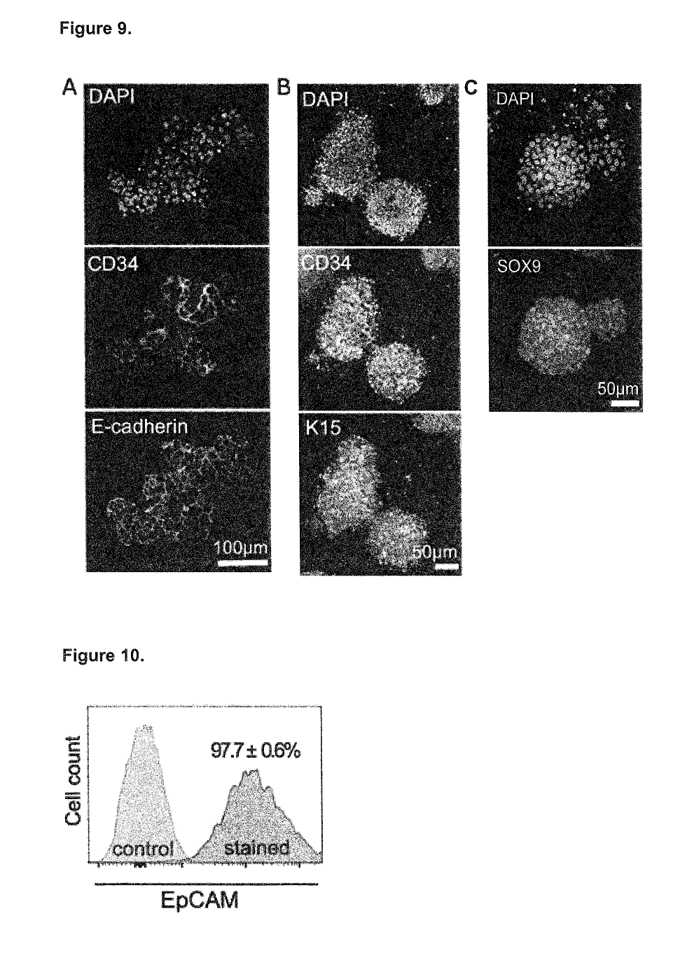

FIG. 9: Expression of stem cell markers is maintained in 3C conditions

A and B. Confocal images of HFSCs grown under 3C conditions and stained for the indicated proteins. K15: Keratin-15. Scale bar 100 μm.

FIG. 10: 3C cultures contain exclusively epithelial cells

Representative FACS histogram of day 14 (d14) HFSC cultures stained for EpCAM and quantified by flow cytometry. Unstained cells are shown as control. Percentage of EpCAM⁺ cells ±SD from 6 independent cultures is shown.

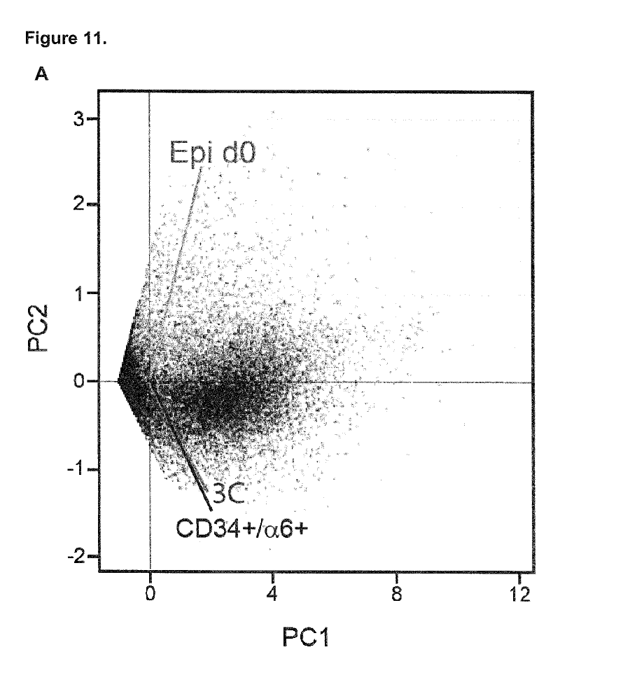

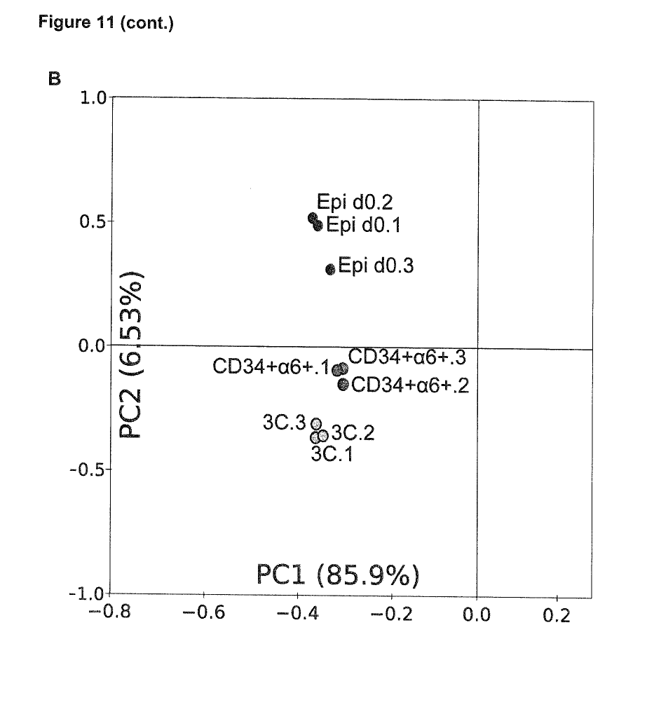

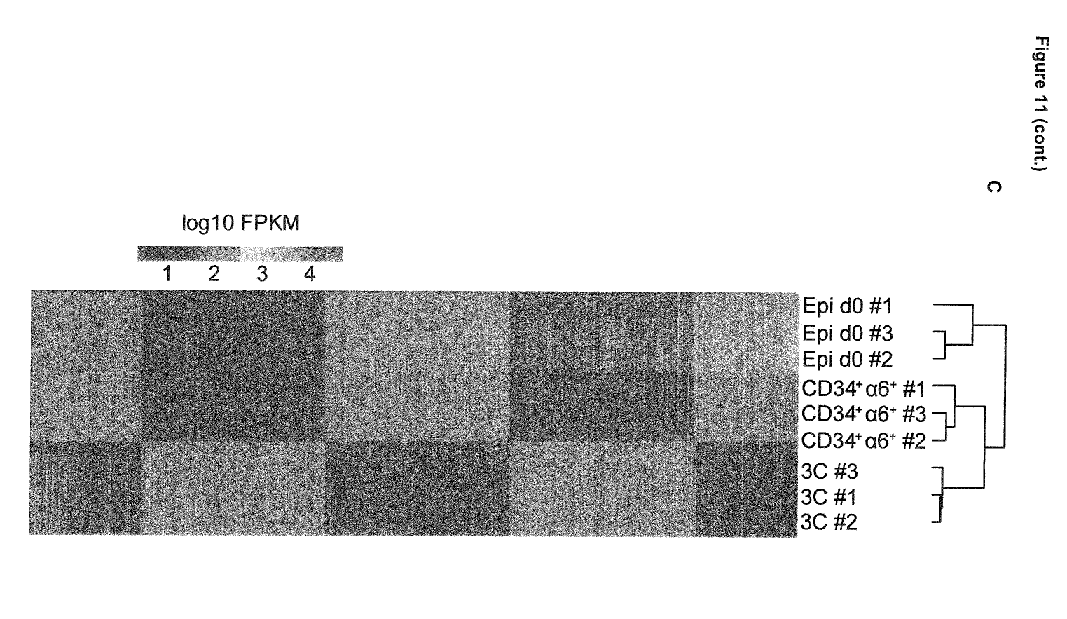

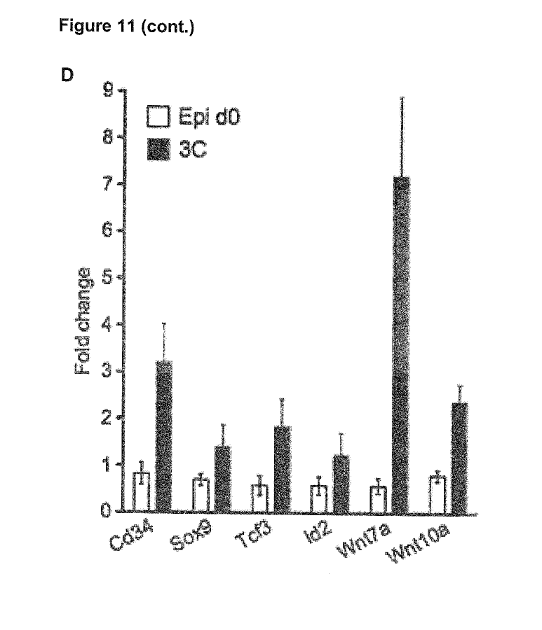

FIG. 11: Transcriptomes of cells grown in 3C cultures resemble transcriptomes of bona fide HFSCs

A. Principal component analysis of the transcriptome obtained from RNA sequencing experiments of i) epidermis-derived cells (Epi d0), ii) FACS-purified CD34⁺α6⁺ HFSCs (CD34⁺α6⁺), and iii) epidermis-derived cells cultured under 3C conditions for 14 days (3C). Purified CD34⁺α6⁺ HFSCs and cells cultured in 3C conditions cluster together and are distinct from epidermis-derived cells indicating that their transcriptomes share significant similarity.

B. Principal component analysis of the transcriptome obtained from RNA sequencing experiments of i) epidermis-derived cells (Epi d0), ii) FACS-purified CD34⁺α6⁺ HFSCs (CD34⁺α6⁺), and iii) epidermis-derived cells cultured under 3C conditions for 14 days (3C). Plot shows individual biological replicates that cluster together indicating good reproducibility. As in panel A, purified CD34⁺α6⁺ HFSCs and cells cultured in 3C conditions cluster together and are distinct from epidermis-derived cells indicating that their transcriptomes share significant similarity.

C. Heat map analysis of quantified transcripts and dendrogram show that the transcriptomes of cells cultured in 3C more closely resemble transcriptomes of CD34⁺α6⁺ HFSCs than transcriptomes of the original cell mixtures (Epi d0). For each condition three biological replicates (1, 2 and 3) are shown. The represented data is derived from the same experiment as A.

D. Quantitative PCR (qPCR) analysis of Epi d0 and 3C shows that cells in 3C conditions upregulate HFSC identity genes (e.g. Cd34, Sox9, Tcf3, Wnt7a).

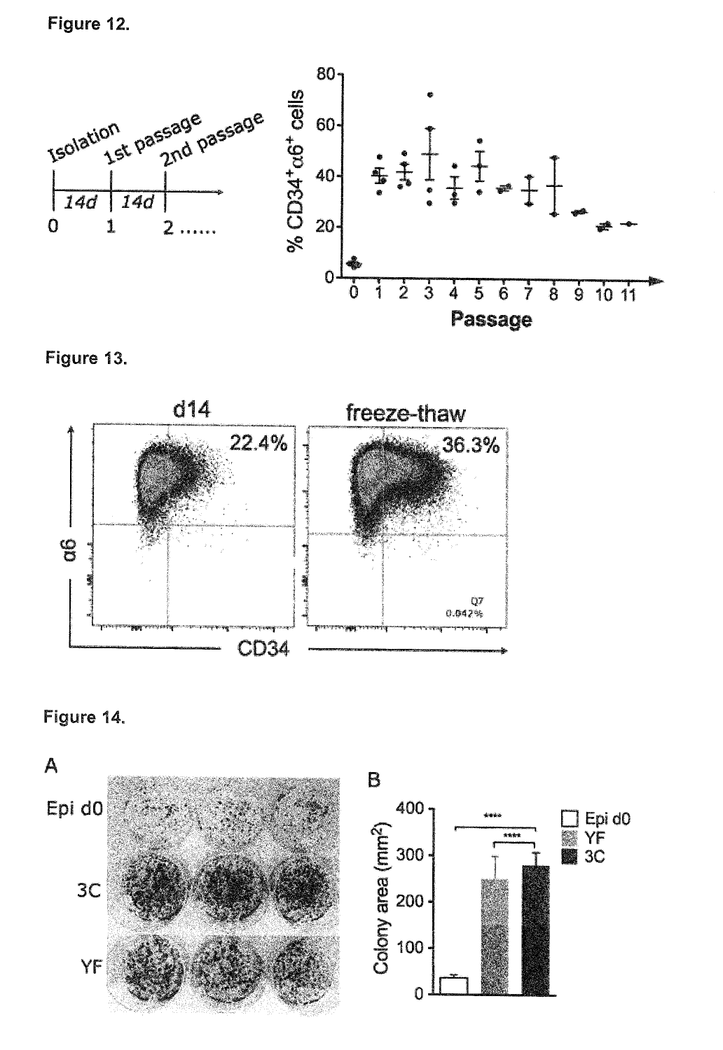

FIG. 12: Long-term passage of 3C cultures

A. Passage scheme. B. Percentage of CD34⁺α6⁺ cells from HFSC cultures quantified by flow cytometry. Individual experiments are depicted as single points. SEM is shown.

FIG. 13: HFSCs cultured under 3C conditions can be freeze-thaw

Representative FACS plots of HFSC cultures before (day 14; d14) and 14 days after freeze-thaw. Cells were frozen for at least a month before thawing them. Thawed cells grew for additional 14 days before analysis.

FIG. 14: In vitro proliferative potential of HFSCs cultured under 3C conditions

Colony forming assays were performed with 3000 cells/well derived from three mice. Representative results (A) and quantification (B) are shown. Freshly isolated epidermal cells were used as a control (mean±SEM; *≥p 0.05, Mann-Whitney U-test). YF: Y27632+EGF+FGF-2; 3C: Y27632+EGF+VEGF+FGF-2. All conditions were performed in triplicates.

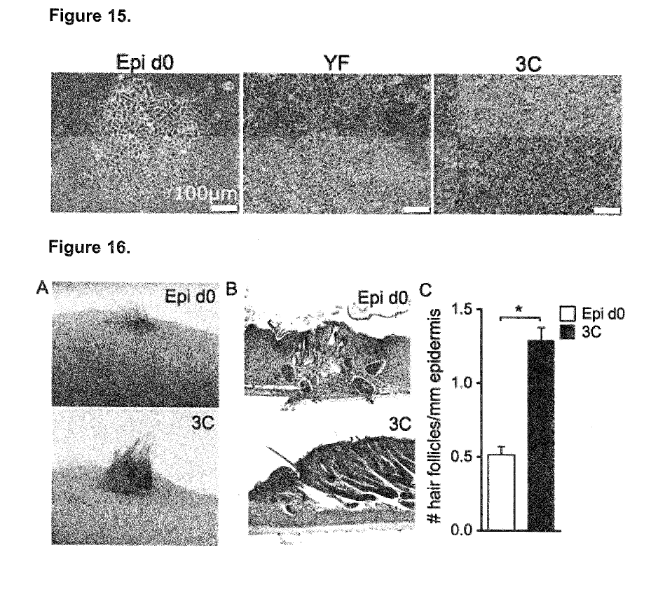

FIG. 15: Morphology of colonies from colony forming assay of HFSCs cultured under 3C conditions

Representative images of characteristic colonies generated by the indicated cells. Scale bar 100 μm. Scale bar 100 μm. YF: Y27632+EGF+FGF-2; 3C: Y27632+EGF+VEGF+FGF-2.

FIG. 16: HFSCs cultured under 3C conditions maintain their multipotency and self-renewal potential in a skin reconstitution assay

A. Representative images of recipient mice transplanted with either freshly isolated epidermal cells (Epi d0) or cultured cells cultured under 3C conditions (3C). Note that animals transplanted with cells cultured in 3C developed more hair than Epi d0 controls indicating their multipotency and self-renewal capacity after 14 days of culture. B. H&E staining from transplant's biopsies. C. Hair follicle quantification from Hematoxylin & Eosin staining.

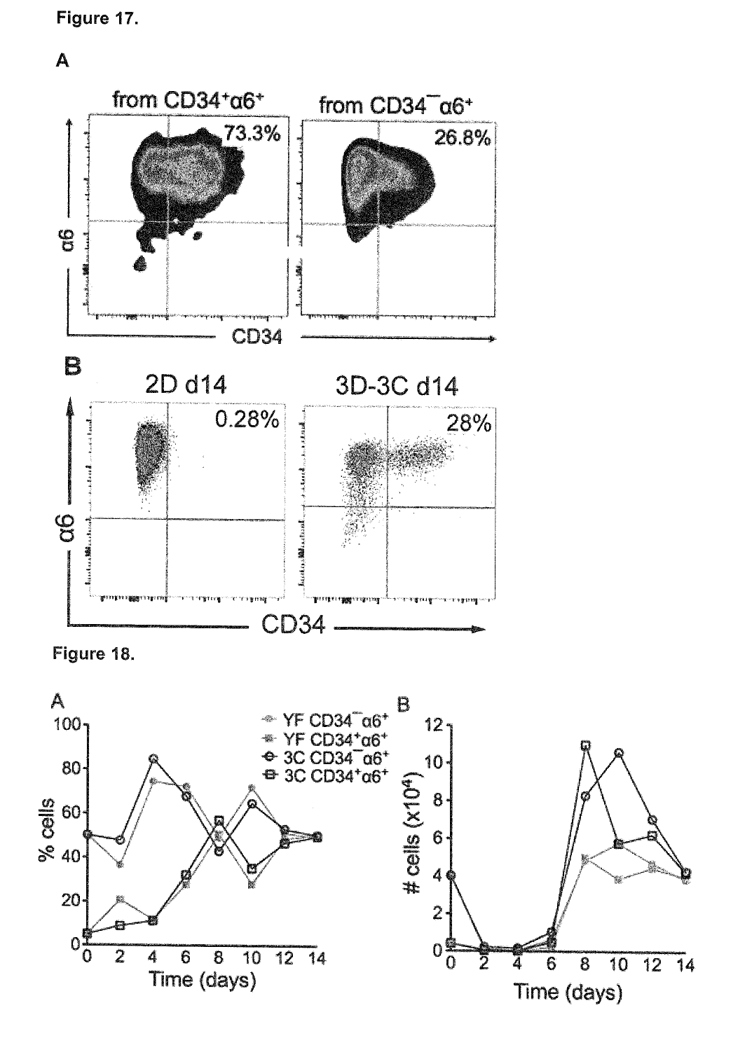

FIG. 17: De novo generation of HFSCs from non-HFSCs under 3C culture conditions

A. CD34⁺α6⁺ HFSCs and CD34⁻α6⁺ non-HFSCs were purified from total epidermis into at least 98% purity and cultured under 3C conditions for 14 days. FACS plots of day 14 cultures established from the indicated purified cell populations show that both cultures consist of a significant population of CD34⁺α6⁺ HFSCs and CD34⁻α6⁺ non-HFSCs.

B. CD34⁺α6⁺ HFSCs were completely depleted by culturing total epidermal cells in 2D growth conditions for 14 days (2D 14 d). The resulting CD34⁻α6⁺ non-HFSCs were subsequently passaged and cultured under 3C conditions for additional 14 days (3D-3C d14). Shown are FACS blots of the indicated cells and the percentage of CD34⁺α6⁺ HFSCs is indicated.

FIG. 18: 3C HFSC cultures reach equilibrium after 12 days of culture

CD34⁺α6⁺ and CD34⁻α6⁺ cells in 3C cultures were monitored every 2 days for a period of 14 days. Cell populations (A) and absolute numbers (B) were quantified by flow cytometry.

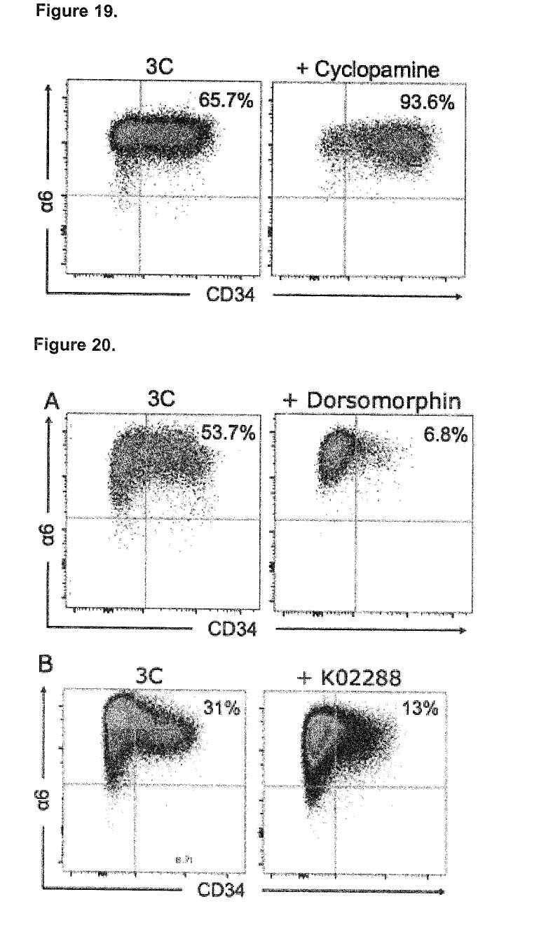

FIG. 19: Inhibition of SHH signaling increases the number of CD34⁺α6⁺ cells in 3C HFSC cultures

Cells were grown for 9 days under 3C conditions and treated with the SHH inhibitor cyclopamine (10 μM) for 5 days. Treated cultures and untreated 3C control cultures were analyzed by flow cytometry on day 14.

FIG. 20: Inhibition of BMP signaling decreases the numbers of CD34⁺α6⁺ cells in 3C HFSC cultures

Cells were grown for 14 days under 3C conditions with the BMP inhibitor dorsomorphin (2 μM) (A) or the BMP inhibitor K02288 (10 nM) (B). Treated cultures and untreated 3C control cultures were analyzed by flow cytometry.

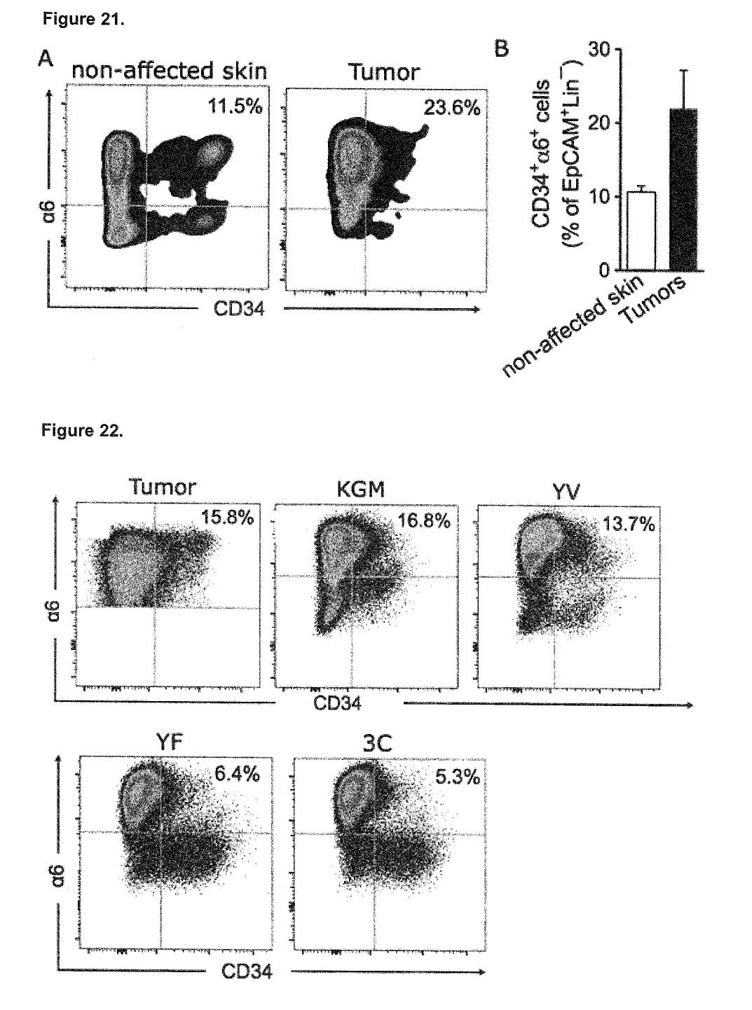

FIG. 21: Papillomas contain phenotypically defined CSCs in a mouse model of skin cancer

Papillomas arising in the K14rTA tet-O-Kras mouse model were stained for CSCs markers defined as Lin⁻EpCAM⁺CD34⁺α6⁺. Representative FACS plots from unaffected skin and tumor material. Gates were drawn according to the respective unstained and isotype-stained controls. Percentages are indicated per quadrant. n>12 tumors; n=5 mice. SEM is shown.

FIG. 22: Papilloma CSCs can be grown and maintained in vitro under different culture conditions

Single cell suspensions prepared from papillomas of the K14rTA tet-O-Kras mouse model were grown using different media that support HFSC growth. A. FACS plots showing CSC staining after 14 days of culture. Tumor: cells isolated from tumors; KGM: keratinocyte growth medium. YV: Y27632+EGF+VEGF; YF: Y27632+EGF+FGF-2; 3C: Y27632+EGF+VEGF+FGF-2.

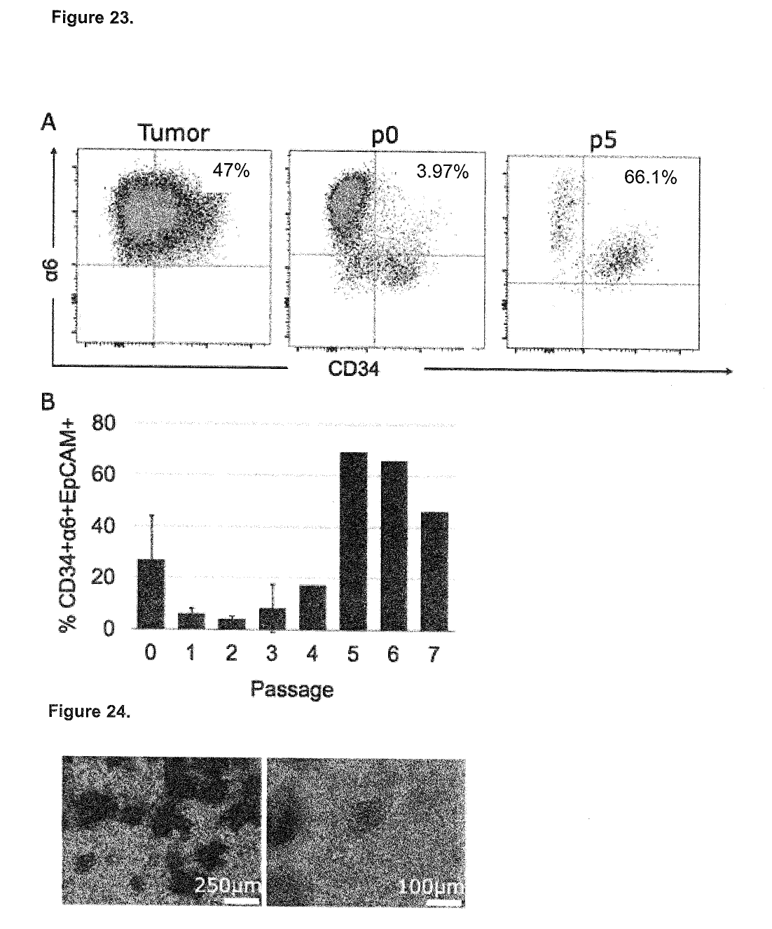

FIG. 23: 3C culture conditions support enrichment of papilloma CSCs in long-term cultures

A. FACS plots of papilloma cells before (Tumor: freshly isolated tumor cells) and after (2 weeks of culture (p0) and 12 weeks of culture (p5)) culture. B. Percentage of EpCAM⁺CD34⁺α6⁺ cells from papilloma CSC cultures quantified by flow cytometry.

FIG. 24: Morphology of papilloma CSCs cultured under 3C conditions

Representative bright field images of papilloma cells grown under 3C conditions for 12 weeks. 5× magnification. Scale bars 100 μm (left) and 250 μm (right).

The invention will now be described by reference to the following examples which are merely illustrative and are not to be construed as a limitation of the scope of the present invention.

### EXAMPLE 1: DEVELOPMENT OF CULTURE CONDITIONS FOR CULTURING HFSCS AND EXPERIMENTAL CHARACTERIZATION OF SUCH CULTIVATED HFCSS

### Materials and Methods

### Mice

Keratinocytes were isolated from C57BL/6 mice on postnatal day (P) 21, unless stated otherwise. 7-9 weeks old BALB/c-nude mice (CAnN.Cg-Foxn1nu/Crl; Charles River, Germany) were used as recipients in transplantation experiments. Primary dermal fibroblasts were isolated from C57BL/6 mice on P2. Animals were housed and maintained according to FELASA guidelines in the animal facility of the Max Planck Institute for Biology of Ageing, Cologne, Germany. All experiments were approved by local authorities.

### Reagents and Antibodies

The following components were used to prepare the basal keratinocyte growth medium (KGM basal medium): MEM (Spinners modification, Sigma), 5 μg/mL Insulin (Sigma), 10 μg/mL Transferrin (Sigma), 10 μM Phosphoethanolamine (Sigma), 10 μM Ethanolamine (Sigma), 0.36 μg/mL Hydrocortisone (Calbiochem), 2 mM Glutamine (Gibco), 100 U/mL Penicillin and 100 μg/mL Streptomycin (Gibco), 8% chelated fetal calf serum (FCS) or fetal bovine serum (FBS) (Gibco).

The following components were used to prepare the FAD medium: 2 parts of DMEM (Gibco) and 1 part of Ham's F12 (Gibco), 2 mM Glutamine (Gibco), 100 U/mL Penicillin and 100 μg/mL Streptomycin (Gibco), 10% chelated FCS or FBS (Gibco), 50 μg/ml Vitamine C (Sodium L-ascorbate; Sigma), 10 ng/ml EGF (Sigma), 5 μg/ml Insulin (Sigma), 0.5 μg/ml Hydrocortisone (Calbiochem, 0.1 nM cholera toxin (Sigma), 180 μM Adenin (Sigma).

Growth factor reduced Matrigel (BD Biosciences), (R)-(+)-trans-4-(1-aminoethyl)-N-(4-Pyridyl)cyclohexanecarboxamide dihydrochloride monohydrate (referred to as Y-27632; Sigma-Aldrich), human recombinant FGF-2, human recombinant FGF-7, human recombinant FGF-10 (all from Miltenyi Biotec), human recombinant FGF-18 (Peprotech) mouse recombinant VEGF-164, human recombinant VEGF-121 (all from Miltenyi Biotec), human recombinant EGF (LONG-EGF; Sigma), rat tail collagen type I (Millipore), human recombinant Laminin-332 and human recombinant Laminin-511 (Biolamina) were used for cell culture experiments. Collagenase (C0130, Sigma) was used for cell recovery from collagen type I gels. 7AAD (eBioscience) and Fixable Viability Dye eFluor405 (eBioscience) were used to discriminate dead cells for flow cytometry.

The following antibodies were used for immunostaining: eFluor660-CD34 (clone RAM34, eBioscience), Pacific blue-alpha-6 Integrin (clone GoH3, eBioscience), FITC-CD133 (clone 13A4, eBioscience), PE-CD200 (clone OX90, eBioscience), PE-Cy7-EpCAM (clone G8.8, eBioscience), PE-CD140a (clone APA5, eBioscience); PE-CD45 (clone 30-F11, eBioscience), PE-CD31 (clone MEC13.3, eBioscience), APC-Cy7-beta-1 Integrin (clone HMB1-1, eBioscience), SOX9 (H-90, Santa Cruz Biotechnology), Keratin15 (LHK15, NeoMarkers, Fremon, Calif.). Secondary antibodies for immunofluorescence anti-mouse AlexaFluor 488 and anti-rabbit AlexaFluor 568 were from LifeTechnologies.

### Cell Isolation and Culture

Keratinocytes were isolated from back skin of mice by incubating skin pieces in 0.8% Trypsin (Gibco) for 50 min at 37° C. After separating the epidermis from the underlying dermis, cells were homogenized, filtered through 70 μm and 45 μm cell strainers (BD Biosciences) and pelleted at 900 rpm for 3 min. For 2D culture of epidermis-derived cells, tissue culture petri dished were coated with a mixture of collagen I (30 μg/ml)+fibronectin (10 μg/ml; both from Millipore) in MEM for 1 h at 37° C. prior to plating of cells. Epidermis-derived cells were suspended in KGM or in FAD medium, and seeded on the coated plastic dishes. Medium was exchanged the next day after initial seeding and thereafter every second day. Cultures were incubated at 37° C., 5% CO₂ for 12-14 days. For 3D culture of HFSCs, 8×10⁴ cells were resuspended in 40 μl ice-cold 1:1 KGM:Matrigel mixture that was dispensed as a droplet in 24-well cell culture dishes. The suspension was allowed to solidify for 15 min after which it was overlaid with 500 μl of stem cell media. The following media compositions were used: KGM basal medium, medium Y (KGM, 5 μM Y27632, 10 ng/ml human recombinant EGF), medium Y-E (KGM, 5 μM Y27632), medium YV (KGM, 5 μM Y27632, 10 ng/ml human recombinant EGF, 20 ng/ml mouse recombinant VEGF), medium YF (KGM, 5 μM Y27632, 10 ng/ml human recombinant EGF, 20 ng/ml human recombinant FGF-2), 3C (KGM, 5 μM Y27632, 10 ng/ml human recombinant EGF, 20 ng/ml mouse recombinant VEGF, 20 ng/ml human recombinant FGF-2), and 3C-E (KGM, 5 μM Y27632, 20 ng/ml mouse recombinant VEGF, 20 ng/ml human recombinant FGF-2). Cultures were incubated at 37° C., 5% CO₂ for 12-14 days during which the medium was replaced every 2 days.

For cell passaging and flow cytometry cultured hair follicle stem cells were extracted from Matrigel by mechanical homogenization and incubation in 0.5% Trypsin (Gibco), 0.5 mM EDTA, PBS or Accutase (Gibco) for 10 min at 37° C. Cells were passaged every 10-14 days in a 1:3 to 1:5 ratio in fresh 1:1 KGM (where necessary with the respective supplements):Matrigel.

### Proliferative Potential Assay

A colony-forming assay to determine proliferative potential of stem and progenitor cells was conducted as previously described (Jensen et al., 2010, Nat Protoc 5: 898-911). Briefly, 2000-4000 cells were plated on 6-well plates containing Mitomycin C-treated feeder cells (J2 fibroblasts). Cultures were incubated at 37° C., 5% CO₂ for 12-14 days during which the medium was replaced every 2 days. Experiments were terminated when colonies reached a sufficient size to be visually identified and quantified. Colonies were fixed with 4% PFA 10 min and then stained with 1% crystal violet. Colony number and area were determined using the ImageJ software. All conditions were performed in triplicates.

### Flow Cytometry (FACS Analysis and FACS Purification)

Single cell suspensions prepared from murine back skin (described above) or from cultured cells were rinsed once with KGM and stained with fluorescently labeled antibodies for 30 min on ice. After two washes with FACS Buffer (2% FCS, 2 mM EDTA, PBS) cells were measured in a BD FACS Canto II and data was analyzed using FlowJo software version 10. For purification of cell populations (cell sorting) a BD FACS Aria II or a BD FACS Aria Fusion were used. Cells were sorted in KGM medium at 4′C. Expression of cell surface markers was analyzed on live cells after exclusion of cell doublets and dead cells, respectively.

### EdU Incorporation Assay

Cells were grown in the presence or absence of 9.4 μm EdU (LifeTechnologies) for 24 or 48 h before analysis. After preparing single-cell suspensions, cells were stained with a fixable viability dye eFluor506 (eBiosciences) followed by antibody staining before fixation in 4% PFA for 10 min at RT. Cells were next permeabilized in 0.1% Triton X-100, PBS for 10 min, and incubated 30 min in EdU reaction cocktail (100 mM Tris pH 8.5, 1 mM CuSO₄, 0.5 μM AlexaFluor-488-Azide (A10266, Life Technologies), 100 mM ascorbic acid). After 2 washes with 0.1% Triton X-100, PBS cells were analyzed by flow cytometry.

### Hematoxylin and Eosin Staining

Paraffin-embedded tissue sections were deparaffinized with Xylol and rehydrated with consecutive washes of 100% isopropanol, 95%, 75%, 50% ethanol, and distilled water. Sections were stained for 1 min with Hematoxylin and counterstained for 10 sec with Eosin. Sections were then dehydrated in 50%, 75%, 95% ethanol and isopropanol, cleared with Xylol, and mounted in Entellan (Merk).

### Immunofluorescence

Cultured hair follicle stem cells were rinsed once in PBS, followed by fixation in 2% PFA, PBS for 30 min at room temperature (RT). Fixed cells were rinsed three times with 100 mM glycine, PBS, then permeabilized and blocked for 2 h at 37° C. in 0.3% Triton-X 100, 5% BSA, PBS. Cells were stained with unlabeled or fluorescent primary antibodies in 0.3% Triton-X 100, 1% BSA, PBS overnight at RT. Secondary, fluorescent antibodies were used to detect primary antibody binding and nuclei were visualized with DAPI. Slides were mounted with Elvanol mounting medium (0.2 M Tris pH 6.5, 12% polyvinyl alcohol, 30% glycerol, 2.5% DABCO-anti fade reagent).

### Microscopy and Image Analysis

Fluorescent images were collected by laser scanning confocal microscopy (TCS SP5X; Leica) with ×63 or ×40 immersion objectives using LAS X software. All images were recorded sequentially and averaged at least twice. Image processing (linear brightness and contrast enhancement) was performed with Fiji Software version 2.0.0 or Adobe Photoshop CSS.

### Full Thickness Skin Reconstitution Assay (Transplantation Assay)

Transplantation of keratinocytes to assess their multipotency and self-renewal capacity was performed essentially as previously described (Blanpain et al., 2004, Cell 118: 635-648; Jensen et al., 2010, Nat Protoc 5: 898-911). Briefly, a mixture of 1-5×10⁵ freshly isolated keratinocytes or cultured cells together with 5×10⁶ freshly isolated neonatal fibroblasts was injected into a silicon chamber inserted into a full thickness wound on the back skin of nude mice (6-8 weeks old). The chamber was removed a week after transplantation and hair growth was monitored for the following 2-4 weeks.

### Real Time Quantitative PCR (RT-qPCR)

RNA was extracted with the RNeasy Plus Mini Kit (Qiagen). cDNA was synthesized with the SuperScript VILO (LifeTechnologies) or the High-Capacity cDNA Reverse Transcription Kit (Applied Biosystems). qPCR was performed on the CFX384 Touch Real Time PCR Detection System (Bio-Rad) and the StepOne Plus Real Time PCR System (Applied Biosystems) with DyNAmo ColorFlash SYBR Green Mix (Thermo Fisher). Gene expression changes were calculated following normalization to an ERCC spike-in reference RNA (Ambion-LifeTechnologies) using the comparative Ct (cycle threshold) method.

### RNA Sequencing

Total RNA was extracted as describe above for RT-qPCR. RNA quality was evaluated with an Agilent 2200 TapeStation. Three biological replicates/condition were sequenced. Libraries were made with NEBNext Ultra Directional RNA Library Prep Kit (New England Biolabs) followed by sequencing with HiSeq 2500 (Illumina).

Reads were mapped to the Mus musculus reference genome (build GRCm38_79), after quality control, followed by differential gene expression analysis using Cufflinks (version 2.2.1). Transcripts regulated ≥2 log 2 fold change and with adjusted p-value≤0.05 were considered significantly regulated. The principal component analysis (PCA) was performed using the cummeRbund package and R software. The data have been submitted to NCBI-GEO (GSE76779).

### Statistical Analysis

Statistical analyses were performed using GraphPad Prism software (GraphPad, version 5.0). Statistical significance was determined by the Mann-Whitney U-test or ANOVA test.

### Results

### Development of Culture Conditions for Expansion and Maintenance Hair Follicle Stem Cells

Murine keratinocytes were cultured employing standard culture conditions. Standard growth conditions are defined by the use of KGM or FAD medium, a widely used standard keratinocyte culture medium (Watt & Green, 1982, Nature 295:434-436), as growth media. In addition the cells are cultured in 2-dimensional (2D) culture conditions, which are achieved by coating a tissue culture petri dish with ECM components (such as Collagen I), allowing them to polymerize and subsequently plating the cells on top of the ECM surface.

Flow cytometry analyses of murine keratinocytes cultivated under these conditions or freshly isolated murine keratinocytes for comparison, demonstrated that the CD34⁺α6⁺ HFSC population was rapidly depleted (e.g. within 14 days) under said standard culture conditions (FIG. 1). Freshly isolated keratinocytes from a P21 mouse contained 5.6±1.2% (±SD) CD34⁺α6⁺ HFSCs (FIG. 1 and FIG. 3).

In order to develop improved in vitro culturing conditions for HFSCs, the role of laminins and collagens in a three-dimensional (3D) ECM microenvironment was assessed. To culture the cells in the presence of a 3D-ECM, single cell suspensions were embedded within the ECM mixture prior to polymerization of the ECM components, allowing the cells to be embedded within the ECM from all sides. The survival and growth/expansion of keratinocytes in 3D ECM gels composed of collagen type I, collagen type I and laminins, and a laminin-rich basement membrane extract (Matrigel (Corning) or Culturex (Amsbio)), was subsequently analyzed.

In particular, freshly isolated keratinocytes were embedded in Matrigel or Culturex (from now on referred to as 3D-Matrigel ECM), and cultured in KGM comprising EGF. Under these conditions no cell growth was observed. Yet, keratinocyte survival and/or growth/expansion could be established in the presence in 3D-Matrigel ECM by adding EGF and a ROCK inhibitor (e.g. Y27632) to the KGM culture medium (FIG. 2 and FIG. 3, see Y). As determined by flow cytometry analyses the population of CD34⁺α6⁺ HFSCs was significantly increased under this culturing condition employing a medium comprising EGF and the ROCK inhibitor Y27632 (termed Y medium; see FIG. 3).

An increased relative contribution of CD34⁺α6⁺ HFSC population within the keratinocyte population in 3D-Matrigel ECM could be achieved by adding FGF-2 and/or VEGF (both mitogenic growth factors) in addition to EGF and the ROCK inhibitor Y27632 to the KGM culture medium (FIG. 2 and FIG. 3). In particular, culturing keratinocytes in 3D-Matrigel ECM and KGM medium comprising Y27632, EGF and FGF-2 (from here on termed YF culturing conditions; the respective medium is referred to as YF medium) or Y27632, EGF and VEGF (termed YV culturing conditions; the respective medium is referred to as YV medium) increased the relative contribution of the CD34⁺α6⁺ HFSC population. However, the YF or YV culturing conditions did not increase the absolute numbers of CD34⁺α6⁺ HFSCs, suggesting that both conditions rather promote HFSCs survival than HFSCs growth/expansion. By contrast, culturing cells in 3D-Matrigel ECM and KGM medium comprising Y27632, EGF, a FGF (e.g. FGF-2) and VEGF (referred from here on as 3C culturing conditions; the respective medium is referred to as 3 C medium) had a significant positive effect not only on the survival but also on the growth/expansion of CD34⁺α6⁺ HFSCs within the cultured keratinocyte population. In particular, ˜6-fold increase in the percentage and ˜5-fold increase in absolute numbers was achieved by the 3C culturing conditions (FIG. 3).

To test whether EGF was required we compared Y and 3C conditions using a basal KGM medium with and without EGF. These culturing conditions employing 3D-Matrigel ECM and a KGM medium with the respective compositions are referred to as Y-E conditions and 3C-E conditions, respectively. The respective media are also referred to as Y-E medium and 3C-E medium, respectively. Interestingly, employing KGM without EGF did not affect the relative contribution of CD34⁺α6⁺ HFSCs within the cultured keratinocyte population (FIG. 3A) but, the absolute numbers of HFSCs decreased by almost half (FIG. 3C). This indicated that although the growth of the CD34⁺α6⁺ HFSCs within the cultured keratinocyte population is achieved in the absence of EGF, its presence supports and enhances the expansion of the CD34⁺α6⁺ HFSCs, and therefore, EGF is a preferred component of the 3C medium that is employed under the 3C culturing conditions.

The presence of ROCK inhibitor (in the present example Y27632) in 3C conditions from the beginning of the culturing time is essential for HFSC growth and expansion (see above). To test whether ROCK inhibitor is indispensable for HFSC expansion in 3C conditions after the initial culturing phase (day 0 to day 4) we compared 3C medium to 3C medium without ROCK inhibitor (3C-Y) after cells had been cultured for either 2 and 4 days in 3C medium. These culturing conditions employing 3D-Matrigel ECM and a KGM medium with the respective compositions are referred to as 30-Y culturing conditions. The respective medium is referred to as 3C-Y medium. Interestingly, removing ROCK inhibitor after 2 or 4 days of culture did not affect the relative amounts of CD34⁺α6⁺ HFSCs within the cultured cell population (FIG. 3E), but it decreased the absolute numbers of HFSCs (FIG. 3F). This indicated that although the growth of the CD34⁺α6⁺ HFSCs within the cultured keratinocyte population can be achieved in the absence of ROCK inhibitor after 2 or 4 days of culture, its presence throughout the 14 days of culture supports and enhances the expansion of the CD34⁺α6⁺ HFSCs, and therefore, a ROCK inhibitor and specifically Y27632 is a preferred component of the 3C medium that is preferably used during the complete culturing time.

Comparable growth/expansion of the CD34⁺α6⁺ HFSCs within the cultured keratinocyte population was achieved when FGF-2 was replaced with FGF-7 or FGF-10 or FGF-18, and when VEGF (VEGF-164) was replaced with VEGF-121 in the 3C, YF and/or YV media (FIG. 4). This shows that different FGFs and/or VEGFs can be employed in the culturing method.

Furthermore, significant growth/expansion of the CD34⁺α6⁺ HFSCs within the cultured keratinocyte population was achieved when the 3D basement membrane extract (Matrigel (Corning) or Culturex (Amsbio)) was replaced with 3D gels composed of collagen type I, collagen type I and laminin-332 or collagen type I and laminin-511, or collagen type I, Laminin-332 and Laminin-511 (FIG. 5). This shows that different 3D-ECMs can be employed in the context of the culturing method.

Flow cytometry analyses demonstrated that keratinocytes cultured in the 3C medium in 3D-Matrigel ECM also expressed the surface marker CD200 (FIG. 6) that marks stem/progenitor cells located in the bulge and hair germ compartments in human and mouse skin (Garza et al., 2011, J Clin Invest. 121(2): 613-622). While 19.9±1.5% of freshly isolated keratinocytes from P21 mice expressed CD200, 60.4±12.1% of the cells grown under 3C culturing conditions were CD200⁺ (3-fold increase). Similar to the fold enrichment of the CD34⁺α6⁺ HFSCs in 3C cultures the absolute numbers of CD200⁺ cells were increased 5-fold (FIG. 6).

Cultures containing HFSCs grew into circular cell clusters, but in contrast to other 3D culture systems for epithelial cells known in the art (Lee et al, 2014, Cell 156: 440-455; Sato et al, 2011, Nature 469: 415-418; Sato et al, 2009, Nature 459: 262-265), they did not generate a lumen (FIG. 7). A preliminary EdU (5-ethynyl-2′-deoxyuridine) incorporation experiment performed with cells from a single mouse revealed that both the CD34⁺α6⁺ HFSCs and the CD34⁻α6⁺ non-HFSCs were actively cycling during a 12- to 14-day culture period under 3C conditions. Having this EdU (5-ethynyl-2′-deoxyuridine) incorporation experiment repeated 7 more times confirmed the previous findings in what the CD34⁺α6⁺ HFSCs are actively cycling during a 10-14-day culture period under 3C conditions (see FIGS. 8B and 8C). The in total 8 replicates (including the single experiment in FIG. 8A) indicate that 60.6% of these CD34⁺α6⁺ HFSCs entered S-phase within 24 h (FIGS. 8B and 8C) whereas the CD34⁻α6⁺ (non-stem cell) population cycled slower (45.4%) entered S-phase within 24 h (see FIGS. 8B and 8C). Accordingly the EdU incorporation experiments confirmed that the CD34⁺α6⁺ HFSCs divide, which is in accordance with the HFSCs being expanded during 3C culturing.

Immunofluorescence analysis of HFSCs cultured under 3C conditions revealed the presence of E-cadherin-containing cell-cell contacts, and the expression of additional HFSC markers including Keratin-15, CD34, and SOX9 (FIG. 9). In addition, the cultures were found to consist of only epithelial cells as 97.7±0.63% were positive for the epithelial cell adhesion molecule (EpCAM) (FIG. 10). RNA sequencing analysis of keratinocytes cultured in the 3C medium in the presence of 3D-Matrigel ECM confirmed that the global gene expression profile of these cells more closely resembled purified HFSCs than the total epidermal cells (see Principle component analyses (PCA) shown in FIGS. 11A and B and Heat map shown in FIG. 11C), supporting the fact that the 3C culturing conditions enrich for HFSCs. In addition to the RNA sequencing analysis, the upregulated expression of several HFSC identity genes (Cd34, Sox9, Tcf3, Id2, Wnt10a, Wnt7a) in cells grown in 3C culturing conditions compared to the cell mixture of origin (Epi d0) was confirmed by real-time qPCR in independent experiments (FIG. 11D).

### Long-Term Culture of HFSCs

HFSCs cultured in 3D-ECM under 3C conditions were passaged every two weeks into fresh 3D-Matrigel ECM for a period of up to 32 weeks with no evident change in their potential to grow/expand and survive. Similarly, the percentage of CD34⁺α6⁺ HFSCs within the cell mixture remained constant from the first passage onward (FIG. 12). Moreover, freeze-thaw experiments demonstrated that cultured HFSCs could be stored frozen and cultured again without evident loss of HFSCs or proliferative capacity (FIG. 13).

HFSCs Cultured Under 3C Conditions Retain their Proliferative Potential and Multipotency

To assess if cultured HFSCs from 3C cultures maintain their proliferative potential and multipotency, colony-forming assays that are the golden standard to quantitatively assess the proliferative potential of SCs (Jensen et al., 2010, Nat Protoc 5: 898-911) were performed. HFSCs (originating from cultivation under 3C conditions) plated on feeders at clonal density gave rise to more colonies that were also larger in size compared to freshly isolated keratinocytes containing 5.6±1.2% HFSCs (FIG. 14). Colonies derived from HFSCs that originated from 3C culturing conditions contained small, tightly packed, cobble stone-like colonies (FIG. 15) that are characteristic for holoclones observed in feeder-dependent 2D-cultures of HFSCs (Blanpain et al., 2004, Cell 118: 635-648; Greco et al., 2009, Cell Stem Cell 4: 155-169). This indicates that the 3C culture system enriches for HFSCs with high proliferative potential.

Full thickness skin reconstitution assays are used to evaluate self-renewal and multipotency of skin SCs (Blanpain et al., 2004, Cell 118: 635-648; Jensen et al., 2010, Nat Protoc 5: 898-911). In this assay, bona fide SCs will give rise to new HFs upon transplantation. HFSCs cultured under 3C conditions not only reconstituted the epidermis and produced hair, but were also more efficient in doing so compared to freshly isolated keratinocyte mixtures containing 5.6±1.2% CD34⁺α6⁺ HFSCs (FIG. 16). Accordingly, this experiment demonstrates that the culture conditions used to expand HFSCs in vitro preserve their multipotency and capacity to self-renew.

HFSC Cultures can be Derived Both from Bona Fide HFSCs as Well as from Committed Progeny

To assess if presence of HFSCs in the initial cell mixture is required for the enrichment and expansion of HFSCs in culture, FACS-sorted HFSCs (CD34⁺α6⁺) or committed epidermal cells (non-HFSCs; CD34″ α6⁺) from freshly isolated keratinocytes were used to establish HFSC cultures. Both cell populations were able to give rise to cultures containing CD34⁺α6⁺ and CD34⁻α6⁺ under 3C culture conditions (FIG. 17A). To further validate these findings, HFSC were completely depleted from cultures by culturing total epidermal cells in 2D for 14 days (2D 14 d) and subsequently sub-culturing (passaging) these cells (non-HFSCs; CD34⁻α6⁺) in 3C conditions. After 14 days in 3C culture conditions HFSCs (CD34⁺α6⁺) arose (FIG. 17B) from CD34⁻α6⁺, demonstrating that the 3C culturing conditions can induce generation of CD34⁺α6⁺ cells from non-HFSC cells. Interestingly, regardless of the number of CD34⁺α6⁺ cells in the initial cell mixtures, the cultures established an equilibrium of approximately 50:50 ratio of HFSCs and non-HFSCs (FIG. 18). This indicates that the 3C culturing conditions are able to induce generation of CD34⁺α6⁺ cells from non-HFSC cells, and on the other hand, to promote differentiation of CD34⁺α6⁺ HFSCs into non-HFSCs, establishing a balance between HFSCs and differentiated progeny in culture.

Addition of SHH Inhibitor to 3C Cultures Increases Proportion of CD34⁺α6⁺ HFSCs

The observation that both CD34⁺α6⁺ HFSCs and their committed progeny established a stable equilibrium in culture (FIG. 18) indicates that dynamic signaling crosstalk between these two populations occurs to establish and maintain this equilibrium under 3C culturing conditions. Surprisingly, further addition of the SHH inhibitor cyclopamine to HFSC 3C cultures led to an even elevated percentage of CD34⁺α6⁺ HFSCs concomitant with a decrease in total cell numbers indicating that SHH regulates the balance between HFSCs and the differentiated progeny (FIG. 19). Accordingly, the addition of SHH can be used to further increase the proportion of CD34⁺α6⁺ HFSCs in 3C HFSC cultures.

Addition of BMP Inhibitors Increases Cell Proliferation and Decreases the CD34⁺α6⁺ HFSCs Population

To evaluate the effect of BMP inhibitors on cultured HFSCs, HFSCs were cultured under 3C conditions in the additional presence of the BMP inhibitors dorsomorphin or K02288. Both BMP inhibitors caused a decrease on the percentage of CD34⁺α6⁺ HFSCs (FIG. 20). Accordingly, addition of BMP inhibitors counteracts the expansion and enrichment of CD34⁺α6⁺ HFSCs observed under 3C conditions.

### EXAMPLE 2: IN VITRO CULTURE CONDITIONS FOR EXPANSION AND MAINTENANCE OF SKIN CANCER STEM CELLS

### Materials and Methods

### Mice

Mice of an age between 4 and 40 weeks were used for experiments. Tumor cells were harvested from Tg(Krt14-rtTA)×Tg(tetO-KRas2) mice (Fisher et al, 2001, Genes Dev 15: 3249-3262; Nguyen et al, 2006, Cell 127: 171-183), where overexpression of mutant KRas was induced by feeding the mice with doxycycline-containing chow (1 g/kg), leading to formation of benign papillomas and squamous cell carcinomas.

Animals were housed and maintained according to FELASA guidelines at the animal facility of the Max Planck Institute for Biology of Ageing, Cologne, Germany. All experiments were approved by local authorities.

### Cell Isolation and Culture

Tumor cells were isolated by mincing tumor biopsies and incubating them in 0.25% collagenase (Sigma), 62.5 U/mL DNaseI (Roche) in HBSS (Hank's Balanced Salt Solution, Gibco) for 60 min with gentle agitation at 37° C. Cell suspensions were filtered through 45 μm strainers and centrifuged at 300×g for 10 min. Single cell suspensions were used for HFSC cultures as describe above. The cells were cultured in identical conditions as described in Example 1 for HFSCs.

Additional materials and methods are described in Example 1.

### Results

### Development of Culture Conditions for Expansion and Maintenance of Epidermis-Derived CSCs

The main challenge to study and manipulate cancer stem cells (CSCs) is the difficulty to obtain these cells in sufficient quantities and the lack of an amenable culture system maintaining the unique cellular properties of these cells. As the culture conditions described above successfully supported maintenance/survival and/or expansion/growth of HFSCs, it was next addressed whether similar conditions could also be used for culturing other epidermis-derived stem cells. In particular, the culturing of epidermis-derived CSC was tested. CSCs from cutaneous papillomas have previously been described to lack linage (Lin) markers (CD140a, CD31, CD45) and to express EpCAM and the markers CD34 and α6 (also expressed by HFSCs). These CSCs account for 20-30% of the total cells in benign papillomas in mouse models (Lapouge et al., 2012, EMBOJ 31: 4563-4575). In accordance with previous studies, FACS analyses indicated presence of 22±16.6% EpCAM⁺CD34⁺α6⁺ cells (hereafter CSCs) in the pool of tumor cells isolated from papillomas obtained from the Tg(Krt14-rtTA)×Tg(tetO-KRas2) mouse model (FIG. 21). Culturing these tumor cells in 2D with standard keratinocyte growth conditions completely depleted the EpCAM⁺CD34⁺α6⁺ population. Culturing these tumor cells under the 3C culturing conditions as described above in Example 1 (e.g. in the presence of Matrigel, Y27632, EGF, an FGF (here FGF-2) and a VEGF) resulted in 97±2% of EpCAM⁺ cells, but the EpCAM⁺CD34⁺α6⁺ population was reduced (6.1±2.5%) after a 2-week culture period (FIG. 22). However, the 3C culturing conditions supported cell growth for more than 5 passages after which the percentage of EpCAM⁺CD34⁺α6⁺ cells had reached 69.3% concomitant with a 6.4-fold increase in absolute cell numbers compared to the number of cells seeded at the beginning of the cultures (FIG. 23). These cultured CSCs grew in clusters similar to the HFSCs (FIG. 24), and could be passaged up to 14 weeks without a significant loss of their growth potential. Moreover, freeze-thaw experiments demonstrated that cultured EpCAM⁺CD34⁺α6⁺ cells could be stored frozen and cultured again without evident loss of EpCAM⁺CD34⁺α6⁺ cell numbers.

1. A method for culturing epidermis-derived stem cells comprising the step of culturing epidermis-derived stem cells in the presence of a three-dimensional extracellular matrix (3D-ECM) and a basal cell culture medium comprising:
Epidermal Growth Factor (EGF); and/or
a Vascular Endothelial Growth Factor (VEGF); and/or
a Fibroblast Growth Factor (FGF);
and further a Rho-kinase (ROCK) inhibitor.

2. The method of claim 1, wherein said basal cell culture medium comprises said EGF, said VEGF, said FGF and said ROCK inhibitor.

3. The method of claim 1 or 2, wherein said Vascular Endothelial Growth Factor (VEGF) is selected from the group consisting of VEGF-164, VEGF-165, VEGF-120 and VEGF-121.

4. The method of any one of claims 1 to 3, wherein said Fibroblast Growth Factor (FGF) is selected from the group consisting of FGF-2, FGF-7, FGF-10 and FGF-18.

5. The method of any one of claims 1 to 4, wherein said ROCK inhibitor is selected from the group consisting of (S)-(+)-2-methyl-1-[(4-methyl-5-isoquinolinyl)sulfonyl]-hexahydro-1H-1,4 diazepine dihydrochloride (H-1152) and (R)-(+)-trans-4-(1-aminoethyl)-N-(4-Pyridyl)cyclohexanecarboxamide dihydrochloride monohydrate (Y-27632).

6. The method of any one of claims 1 to 5, wherein said basal cell culture medium further comprises a Sonic Hedgehog (SHH) inhibitor.

7. The method of any one of claims 1 to 6, wherein said basal cell culture medium further comprises ethanolamine, phospho-ethanolamine and/or transferrin.

8. The method of any one of claims 1 to 7, wherein said epidermis-derived stem cells are hair follicle stem cells (HFSCs) or cancer stem cells (CSCs).

9. The method of any one of claims 1 to 8, wherein said epidermis-derived stem cells are comprised in a mixture of cell types, which further comprises at least one differentiated epidermal cell type.

10. The method of any one of claims 1 to 9, wherein said epidermis-derived stem cells are de novo generated ex vivo.

11. A method for ex vivo de novo generation of epidermis-derived stem cells comprising the step of culturing epidermal cells lacking said epidermis-derived stem cells in the presence of a three-dimensional extracellular matrix (3D-ECM) and a basal cell culture medium as defined in any one of claims 1 to 7 for at least 2 days, at least 4 days, at least 6 days, at least 8 days, at least 10 days, at least 12 days, at least 14 days, at least 16 days, at least 18 days, at least 20 days, at least 100 days, at least 200 days or at least 360 days.

12. An epidermis-derived stem cell that is obtainable by a method as defined in any one of the claims 1 to 11.

13. The epidermis-derived stem cell of claim 12 for use in tissue transplantation and/or for use in treatment of dermal burn, treatment of conditions where areas of skin have been removed due to surgical operation, biopsy, burn and/or trauma, and/or in treatment of conditions where the regenerative capacity of the skin is compromised such as chronic wounds and/or baldness.

14. Use of the epidermis-derived stem cell of claim 12 for in vitro tissue production and/or for in vitro drug discovery screening.

15. A cell culture medium as defined in any one of claims 1 to 7.
